SEQUENCE LISTING (178 sequences)

SEQ ID NO: 1 - DNA, 55 bp, Artificial Sequence
  synthetic oligonucleotide
gcctgctgctacccgtggatattttaaaaaaaaaaaaaaaaaaaaaaaaaaaaaa

SEQ ID NO: 2 - DNA, 60 bp, Artificial Sequence
  synthetic oligonucleotide
ctaccagggtctctaatcctgttggatttaaaaaaaaaaaaaaaaaaaaaaaaaaaaaaa

SEQ ID NO: 3 - DNA, 57 bp, Artificial Sequence
  synthetic oligonucleotide
aatcaacgctagacaggtcaaccctttaaaaaaaaaaaaaaaaaaaaaaaaaaaaaa

SEQ ID NO: 4 - DNA, 52 bp, Artificial Sequence
  synthetic oligonucleotide
aatttaatacgactcactatagggagataaccgaaggacttcggcaaagtaa

SEQ ID NO: 5 - DNA, 46 bp, Artificial Sequence
  synthetic oligonucleotide
aatttaatacgactcactatagggagagctaccctcttccacctgc

SEQ ID NO: 6 - DNA, 49 bp, Artificial Sequence
  synthetic oligonucleotide
aatttaatacgactcactatagggagaggcatcacggacctgttattgc

SEQ ID NO: 7 - DNA, 54 bp, Artificial Sequence
  synthetic oligonucleotide
gcaataatttaatacgactcactatagggagaggcatcacggacctgttattgc

SEQ ID NO: 8 - DNA, 55 bp, Artificial Sequence
  synthetic oligonucleotide
gcaataaatttaatacgactcactatagggagaggcatcacggacctgttattgc

SEQ ID NO: 9 - DNA, 50 bp, Artificial Sequence
  synthetic oligonucleotide
aatttaatacgactcactatagggagagctcgcagtcctattgatcctaa

SEQ ID NO: 10 - DNA, 45 bp, Artificial Sequence
  synthetic oligonucleotide
aatttaatacgactcactatagggagagcaccctctcaggctcgc

SEQ ID NO: 11 - DNA, 49 bp, Artificial Sequence
  synthetic oligonucleotide
aatttaatacgactcactatagggagagtagcgcaccctctcaggctcg

SEQ ID NO: 12 - DNA, 50 bp, Artificial Sequence
  synthetic oligonucleotide
aatttaatacgactcactatagggagagttcatgacgctgattacaaacg

SEQ ID NO: 13 - DNA, 25 bp, Artificial Sequence
  synthetic oligonucleotide
ggcttcgggtctttcaggatattgt

SEQ ID NO: 14 - DNA, 20 bp, Artificial Sequence
  synthetic oligonucleotide
cgggtctttcaggatattgt

SEQ ID NO: 15 - DNA, 22 bp, Artificial Sequence
  synthetic oligonucleotide
gctaacgagcgagattatcgcc

SEQ ID NO: 16 - DNA, 21 bp, Artificial Sequence
  synthetic oligonucleotide
ggtagcaataacaggtccgtg

SEQ ID NO: 17 - DNA, 22 bp, Artificial Sequence
  synthetic oligonucleotide
ggtccgtgatgccctttagatg

SEQ ID NO: 18 - DNA, 23 bp, Artificial Sequence
  synthetic oligonucleotide
cgtgatgccctttagatgctctg

SEQ ID NO: 19 - DNA, 24 bp, Artificial Sequence
  synthetic oligonucleotide
cgtgatgccctttagatgctctgg

SEQ ID NO: 20 - RNA, 19 nt, Artificial Sequence
  synthetic oligonucleotide
gccguugguggugcacggc

SEQ ID NO: 21 - RNA, 17 nt, Artificial Sequence
  synthetic oligonucleotide
gcguugauucagcacgc

SEQ ID NO: 22 - RNA, 27 nt, Artificial Sequence
  synthetic oligonucleotide
cgaaguccuucgguuaaaguuccuucg

SEQ ID NO: 23 - RNA, 28 nt, Artificial Sequence
  synthetic oligonucleotide
cgaaguccuucgguuaaaguucacuucg

SEQ ID NO: 24 - RNA, 27 nt, Artificial Sequence
  synthetic oligonucleotide
cgaaguccuucgguuaaaguuccuucg

SEQ ID NO: 25 - RNA, 30 nt, Artificial Sequence
  synthetic oligonucleotide
uucgguuaaaguucuaauugggacuccgaa

SEQ ID NO: 26 - RNA, 30 nt, Artificial Sequence
  synthetic oligonucleotide
uucgguuaaaguucuaauugggacaccgaa

SEQ ID NO: 27 - RNA, 27 nt, Artificial Sequence
  synthetic oligonucleotide
gcgugcuacaauguuaggaucacacgc

SEQ ID NO: 28 - RNA, 26 nt, Artificial Sequence
  synthetic oligonucleotide
gacugcgagccugagagggugacguc

SEQ ID NO: 29 - DNA, 50 bp, Artificial Sequence
  synthetic oligonucleotide
gatggagcgtaccaccgtttaaaaaaaaaaaaaaaaaaaaaaaaaaaaaa

SEQ ID NO: 30 - DNA, 53 bp, Artificial Sequence
  synthetic oligonucleotide
agatcggtatcgggtgcttgtttaaaaaaaaaaaaaaaaaaaaaaaaaaaaaa

SEQ ID NO: 31 - DNA, 59 bp, Artificial Sequence
  synthetic oligonucleotide
gctcagaaaaccagaagcgaaacgggtttaaaaaaaaaaaaaaaaaaaaaaaaaaaaaa

SEQ ID NO: 32 - DNA, 46 bp, Artificial Sequence
  synthetic oligonucleotide
aatttaatacgactcactatagggagatcaagttcgcatattgcac

SEQ ID NO: 33 - DNA, 49 bp, Artificial Sequence
  synthetic oligonucleotide
aatttaatacgactcactatagggagaatactgggccgacatccttacg

SEQ ID NO: 34 - DNA, 21 bp, Artificial Sequence
  synthetic oligonucleotide
ggtagtttggcttttctttgg

SEQ ID NO: 35 - DNA, 21 bp, Artificial Sequence
  synthetic oligonucleotide
cgttacaagaaatatacacgg

SEQ ID NO: 36 - DNA, 18 bp, Artificial Sequence
  synthetic oligonucleotide
gcattggagtttctgctg

SEQ ID NO: 37 - RNA, 22 nt, Artificial Sequence
  synthetic oligonucleotide
ggaugugacugucaugccaucc

SEQ ID NO: 38 - RNA, 28 nt, Artificial Sequence
  synthetic oligonucleotide
ggaauggcgccguggaugguugcauucc

SEQ ID NO: 39 - DNA, 22 bp, Artificial Sequence
  synthetic oligonucleotide
gcctgctgctacccgtggatat

SEQ ID NO: 40 - DNA, 26 bp, Artificial Sequence
  synthetic oligonucleotide
ctaccagggtctctaatcctgttgga

SEQ ID NO: 41 - DNA, 24 bp, Artificial Sequence
  synthetic oligonucleotide
aatcaacgctagacaggtcaaccc

SEQ ID NO: 42 - DNA, 25 bp, Artificial Sequence
  synthetic oligonucleotide
taaccgaaggacttcggcaaagtaa

SEQ ID NO: 43 - DNA, 19 bp, Artificial Sequence
  synthetic oligonucleotide
gctaccctcttccacctgc

SEQ ID NO: 44 - DNA, 22 bp, Artificial Sequence
  synthetic oligonucleotide
ggcatcacggacctgttattgc

SEQ ID NO: 45 - DNA, 23 bp, Artificial Sequence
  synthetic oligonucleotide
gctcgcagtcctattgatcctaa

SEQ ID NO: 46 - DNA, 18 bp, Artificial Sequence
  synthetic oligonucleotide
gcaccctctcaggctcgc

SEQ ID NO: 47 - DNA, 22 bp, Artificial Sequence
  synthetic oligonucleotide
gtagcgcaccctctcaggctcg

SEQ ID NO: 48 - DNA, 23 bp, Artificial Sequence
  synthetic oligonucleotide
gttcatgacgctgattacaaacg

SEQ ID NO: 49 - DNA, 25 bp, Artificial Sequence
  synthetic oligonucleotide
ggcttcgggtctttcaggatattgt

SEQ ID NO: 50 - DNA, 20 bp, Artificial Sequence
  synthetic oligonucleotide
cgggtctttcaggatattgt

SEQ ID NO: 51 - DNA, 22 bp, Artificial Sequence
  synthetic oligonucleotide
gctaacgagcgagattatcgcc

SEQ ID NO: 52 - DNA, 21 bp, Artificial Sequence
  synthetic oligonucleotide
ggtagcaataacaggtccgtg

SEQ ID NO: 53 - DNA, 22 bp, Artificial Sequence
  synthetic oligonucleotide
ggtccgtgatgccctttagatg

SEQ ID NO: 54 - DNA, 23 bp, Artificial Sequence
  synthetic oligonucleotide
cgtgatgccctttagatgctctg

SEQ ID NO: 55 - DNA, 24 bp, Artificial Sequence
  synthetic oligonucleotide
cgtgatgccctttagatgctctgg

SEQ ID NO: 56 - RNA, 14 nt, Artificial Sequence
  synthetic oligonucleotide
gccguugguggugc

SEQ ID NO: 57 - RNA, 13 nt, Artificial Sequence
  synthetic oligonucleotide
gcguugauucagc

SEQ ID NO: 58 - RNA, 22 nt, Artificial Sequence
  synthetic oligonucleotide
cgaaguccuucgguuaaaguuc

SEQ ID NO: 59 - RNA, 25 nt, Artificial Sequence
  synthetic oligonucleotide
uucgguuaaaguucuaauugggacu

SEQ ID NO: 60 - RNA, 24 nt, Artificial Sequence
  synthetic oligonucleotide
uucgguuaaaguucuaauugggac

SEQ ID NO: 61 - RNA, 22 nt, Artificial Sequence
  synthetic oligonucleotide
gcgugcuacaauguuaggauca

SEQ ID NO: 62 - RNA, 21 nt, Artificial Sequence
  synthetic oligonucleotide
gacugcgagccugagagggug

SEQ ID NO: 63 - RNA, 23 nt, Artificial Sequence
  synthetic oligonucleotide
gcauggugcgaauugggacaugc

SEQ ID NO: 64 - RNA, 26 nt, Artificial Sequence
  synthetic oligonucleotide
gaagguuacuuugccgaaguccuucg

SEQ ID NO: 65 - DNA, 27 bp, Artificial Sequence
  synthetic oligonucleotide
aatttaatacgactcactatagggaga

SEQ ID NO: 66 - DNA, 28 bp, Artificial Sequence
  synthetic oligonucleotide
gaaattaatacgactcactatagggaga

SEQ ID NO: 67 - DNA, 32 bp, Artificial Sequence
  synthetic oligonucleotide
ttgccgaagtccttcggttaaagttctaattg

SEQ ID NO: 68 - RNA, 32 nt, Artificial Sequence
  synthetic oligonucleotide
uugccgaaguccuucgguuaaaguucuaauug

SEQ ID NO: 69 - DNA, 32 bp, Artificial Sequence
  synthetic oligonucleotide
caattagaactttaaccgaaggacttcggcaa

SEQ ID NO: 70 - RNA, 32 nt, Artificial Sequence
  synthetic oligonucleotide
caauuagaacuuuaaccgaaggacuucggcaa

SEQ ID NO: 71 - DNA, 32 bp, Artificial Sequence
  synthetic oligonucleotide
tgccgaagtccttcggttaaagttctaattgg

SEQ ID NO: 72 - RNA, 32 nt, Artificial Sequence
  synthetic oligonucleotide
ugccgaaguccuucgguuaaaguucuaauugg

SEQ ID NO: 73 - DNA, 32 bp, Artificial Sequence
  synthetic oligonucleotide
ccaattagaactttaaccgaaggacttcggca

SEQ ID NO: 74 - RNA, 32 nt, Artificial Sequence
  synthetic oligonucleotide
ccaauuagaacuuuaaccgaaggacuucggca

SEQ ID NO: 75 - DNA, 32 bp, Artificial Sequence
  synthetic oligonucleotide
gccgaagtccttcggttaaagttctaattggg

SEQ ID NO: 76 - RNA, 32 nt, Artificial Sequence
  synthetic oligonucleotide
gccgaaguccuucgguuaaaguucuaauuggg

SEQ ID NO: 77 - DNA, 32 bp, Artificial Sequence
  synthetic oligonucleotide
cccaattagaactttaaccgaaggacttcggc

SEQ ID NO: 78 - RNA, 32 nt, Artificial Sequence
  synthetic oligonucleotide
cccaauuagaacuuuaaccgaaggacuucggc

SEQ ID NO: 79 - DNA, 31 bp, Artificial Sequence
  synthetic oligonucleotide
ccgaagtccttcggttaaagttctaattggg

SEQ ID NO: 80 - RNA, 31 nt, Artificial Sequence
  synthetic oligonucleotide
ccgaaguccuucgguuaaaguucuaauuggg

SEQ ID NO: 81 - DNA, 31 bp, Artificial Sequence
  synthetic oligonucleotide
cccaattagaactttaaccgaaggacttcgg

SEQ ID NO: 82 - RNA, 31 nt, Artificial Sequence
  synthetic oligonucleotide
cccaauuagaacuuuaaccgaaggacuucgg

SEQ ID NO: 83 - DNA, 32 bp, Artificial Sequence
  synthetic oligonucleotide
cgaagtccttcggttaaagttctaattgggac

SEQ ID NO: 84 - RNA, 32 nt, Artificial Sequence
  synthetic oligonucleotide
cgaaguccuucgguuaaaguucuaauugggac

SEQ ID NO: 85 - DNA, 32 bp, Artificial Sequence
  synthetic oligonucleotide
gtcccaattagaactttaaccgaaggacttcg

SEQ ID NO: 86 - RNA, 32 nt, Artificial Sequence
  synthetic oligonucleotide
gucccaauuagaacuuuaaccgaaggacuucg

SEQ ID NO: 87 - DNA, 32 bp, Artificial Sequence
  synthetic oligonucleotide
  misc_feature (8)..(8): n is a, c, g, or t
cgaagtcnttcggttaaagttctaattgggac

SEQ ID NO: 88 - RNA, 32 nt, Artificial Sequence
  synthetic oligonucleotide
  misc_feature (8)..(8): n is a, c, g, or u
cgaagucnuucgguuaaaguucuaauugggac

SEQ ID NO: 89 - DNA, 32 bp, Artificial Sequence
  synthetic oligonucleotide
  misc_feature (25)..(25): n is a, c, g, or t
gtcccaattagaactttaaccgaangacttcg

SEQ ID NO: 90 - RNA, 32 nt, Artificial Sequence
  synthetic oligonucleotide
  misc_feature (25)..(25): n is a, c, g, or u
gucccaauuagaacuuuaaccgaangacuucg

SEQ ID NO: 91 - DNA, 24 bp, Artificial Sequence
  synthetic oligonucleotide
gaagtccttcggttaaagttctaa

SEQ ID NO: 92 - RNA, 24 nt, Artificial Sequence
  synthetic oligonucleotide
gaaguccuucgguuaaaguucuaa

SEQ ID NO: 93 - DNA, 24 bp, Artificial Sequence
  synthetic oligonucleotide
ttagaactttaaccgaaggacttc

SEQ ID NO: 94 - RNA, 24 nt, Artificial Sequence
  synthetic oligonucleotide
uuagaacuuuaaccgaaggacuuc

SEQ ID NO: 95 - DNA, 25 bp, Artificial Sequence
  synthetic oligonucleotide
gtccttcggttaaagttctaattgg

SEQ ID NO: 96 - RNA, 25 nt, Artificial Sequence
  synthetic oligonucleotide
guccuucgguuaaaguucuaauugg

SEQ ID NO: 97 - DNA, 25 bp, Artificial Sequence
  synthetic oligonucleotide
ccaattagaactttaaccgaaggac

SEQ ID NO: 98 - RNA, 25 nt, Artificial Sequence
  synthetic oligonucleotide
ccaauuagaacuuuaaccgaaggac

SEQ ID NO: 99 - DNA, 32 bp, Artificial Sequence
  synthetic oligonucleotide
ttcggttaaagttctaattgggactccctgcg

SEQ ID NO: 100 - RNA, 32 nt, Artificial Sequence
  synthetic oligonucleotide
uucgguuaaaguucuaauugggacucccugcg

SEQ ID NO: 101 - DNA, 32 bp, Artificial Sequence
  synthetic oligonucleotide
cgcagggagtcccaattagaactttaaccgaa

SEQ ID NO: 102 - RNA, 32 nt, Artificial Sequence
  synthetic oligonucleotide
cgcagggagucccaauuagaacuuuaaccgaa

SEQ ID NO: 103 - DNA, 44 bp, Artificial Sequence
  synthetic oligonucleotide
ttgccgaagtccttcggttaaagttctaattgggactccctgcg

SEQ ID NO: 104 - RNA, 44 nt, Artificial Sequence
  synthetic oligonucleotide
uugccgaaguccuucgguuaaaguucuaauugggacucccugcg

SEQ ID NO: 105 - DNA, 44 bp, Artificial Sequence
  synthetic oligonucleotide
cgcagggagtcccaattagaactttaaccgaaggacttcggcaa

SEQ ID NO: 106 - RNA, 44 nt, Artificial Sequence
  synthetic oligonucleotide
cgcagggagucccaauuagaacuuuaaccgaaggacuucggcaa

SEQ ID NO: 107 - DNA, 17 bp, Artificial Sequence
  synthetic oligonucleotide
ttcggttaaagttctaa

SEQ ID NO: 108 - RNA, 17 nt, Artificial Sequence
  synthetic oligonucleotide
uucgguuaaaguucuaa

SEQ ID NO: 109 - DNA, 17 bp, Artificial Sequence
  synthetic oligonucleotide
ttagaactttaaccgaa

SEQ ID NO: 110 - RNA, 17 nt, Artificial Sequence
  synthetic oligonucleotide
uuagaacuuuaaccgaa

SEQ ID NO: 111 - DNA, 22 bp, Artificial Sequence
  synthetic oligonucleotide
gctaacgagcgagattatcgcc

SEQ ID NO: 112 - RNA, 22 nt, Artificial Sequence
  synthetic oligonucleotide
gcuaacgagcgagauuaucgcc

SEQ ID NO: 113 - DNA, 22 bp, Artificial Sequence
  synthetic oligonucleotide
ggcgataatctcgctcgttagc

SEQ ID NO: 114 - RNA, 22 nt, Artificial Sequence
  synthetic oligonucleotide
ggcgauaaucucgcucguuagc

SEQ ID NO: 115 - DNA, 22 bp, Artificial Sequence
  synthetic oligonucleotide
ggcatcacggacctgttattgc

SEQ ID NO: 116 - RNA, 22 nt, Artificial Sequence
  synthetic oligonucleotide
ggcaucacggaccuguuauugc

SEQ ID NO: 117 - DNA, 22 bp, Artificial Sequence
  synthetic oligonucleotide
gcaataacaggtccgtgatgcc

SEQ ID NO: 118 - RNA, 22 nt, Artificial Sequence
  synthetic oligonucleotide
gcaauaacagguccgugaugcc

SEQ ID NO: 119 - DNA, 49 bp, Artificial Sequence; identical to SEQ ID NO: 6
  synthetic oligonucleotide

SEQ ID NO: 120 - DNA, 27 bp, Artificial Sequence
  synthetic oligonucleotide
aatttaatacgactcactatagggaga

SEQ ID NO: 121 - DNA, 22 bp, Artificial Sequence
  synthetic oligonucleotide
gcctgctgctacccgtggatat

SEQ ID NO: 122 - RNA, 22 nt, Artificial Sequence
  synthetic oligonucleotide
gccugcugcuacccguggauau

SEQ ID NO: 123 - DNA, 22 bp, Artificial Sequence
  synthetic oligonucleotide
atatccacgggtagcagcaggc

SEQ ID NO: 124 - RNA, 22 nt, Artificial Sequence
  synthetic oligonucleotide
auauccacggguagcagcaggc

SEQ ID NO: 125 - DNA, 55 bp, Artificial Sequence; identical to SEQ ID NO: 1
  synthetic oligonucleotide

SEQ ID NO: 126 - DNA, 34 bp, Artificial Sequence
  synthetic oligonucleotide
tttaaaaaaaaaaaaaaaaaaaaaaaaaaaaaaa

SEQ ID NO: 127 - DNA, 44 bp, Artificial Sequence
  synthetic oligonucleotide
gctaacgagcgagattatcgccaagcaataacaggtccgtgatg

SEQ ID NO: 128 - DNA, 36 bp, Artificial Sequence
  synthetic oligonucleotide
gtcccaattagaactttaaccgaaggacttcggcaa

SEQ ID NO: 129 - DNA, 32 bp, Artificial Sequence; identical to SEQ ID NO: 101
  synthetic oligonucleotide

SEQ ID NO: 130 - DNA, 21 bp, Artificial Sequence
  synthetic oligonucleotide
caattagaactttaaccgaag

SEQ ID NO: 131 - DNA, 23 bp, Artificial Sequence
  synthetic oligonucleotide
ttgcttggcgataatctcgctcg

SEQ ID NO: 132 - DNA, 27 bp, Artificial Sequence
  synthetic oligonucleotide
cctgttattgcttggcgataatctcgc

SEQ ID NO: 133 - DNA, 29 bp, Artificial Sequence
  synthetic oligonucleotide
cggacctgttattgcttggcgataatctc

SEQ ID NO: 134 - DNA, 24 bp, Artificial Sequence
  synthetic oligonucleotide
gcctctcggctttgcagtcctatt

SEQ ID NO: 135 - DNA, 15 bp, Artificial Sequence
  synthetic oligonucleotide
gttgatcctgccaag

SEQ ID NO: 136 - DNA, 16 bp, Artificial Sequence
  synthetic oligonucleotide
gccatgcaagtgttag

SEQ ID NO: 137 - DNA, 22 bp, Artificial Sequence
  synthetic oligonucleotide
ccattcgactgagtgacctatc

SEQ ID NO: 138 - DNA, 21 bp, Artificial Sequence
  synthetic oligonucleotide
gattcctggttcatgacgctg

SEQ ID NO: 139 - DNA, 16 bp, Artificial Sequence
  synthetic oligonucleotide
ccgagtcatccaatcg

SEQ ID NO: 140 - DNA, 22 bp, Artificial Sequence
  synthetic oligonucleotide
cctaccgttaccttgttacgac

SEQ ID NO: 141 - RNA, 24 nt, Artificial Sequence
  synthetic oligonucleotide
gaaguccuucgguuaaaguucuaa

SEQ ID NO: 142 - RNA, 25 nt, Artificial Sequence
  synthetic oligonucleotide
guccuucgguuaaaguucuaauugg

SEQ ID NO: 143 - DNA, 30 bp, Artificial Sequence
  synthetic oligonucleotide
gtgcgtgggttgacctgtctagcgttgatt

SEQ ID NO: 144 - RNA, 30 nt, Artificial Sequence
  synthetic oligonucleotide
gugcguggguugaccugucuagcguugauu

SEQ ID NO: 145 - DNA, 30 bp, Artificial Sequence
  synthetic oligonucleotide
aatcaacgctagacaggtcaacccacgcac

SEQ ID NO: 146 - RNA, 30 nt, Artificial Sequence
  synthetic oligonucleotide
aaucaacgcuagacaggucaacccacgcac

SEQ ID NO: 147 - DNA, 10 bp, Artificial Sequence
  synthetic oligonucleotide
gacctgtcta

SEQ ID NO: 148 - RNA, 10 nt, Artificial Sequence
  synthetic oligonucleotide
gaccugucua

SEQ ID NO: 149 - DNA, 10 bp, Artificial Sequence
  synthetic oligonucleotide
tagacaggtc

SEQ ID NO: 150 - RNA, 10 nt, Artificial Sequence
  synthetic oligonucleotide
uagacagguc

SEQ ID NO: 151 - DNA, 22 bp, Artificial Sequence
  synthetic oligonucleotide
ctagacaggtcaacccacgcac

SEQ ID NO: 152 - RNA, 22 nt, Artificial Sequence
  synthetic oligonucleotide
cuagacaggucaacccacgcac

SEQ ID NO: 153 - DNA, 22 bp, Artificial Sequence
  synthetic oligonucleotide
gtgcgtgggttgacctgtctag

SEQ ID NO: 154 - RNA, 22 nt, Artificial Sequence
  synthetic oligonucleotide
gugcguggguugaccugucuag

SEQ ID NO: 155 - DNA, 55 bp, Artificial Sequence
  synthetic oligonucleotide
cuagacaggucaacccacgcactttaaaaaaaaaaaaaaaaaaaaaaaaaaaaaa

SEQ ID NO: 156 - DNA, 24 bp, Artificial Sequence
  synthetic oligonucleotide
aatcaacgctagacaggtcaaccc

SEQ ID NO: 157 - RNA, 24 nt, Artificial Sequence
  synthetic oligonucleotide
aaucaacgcuagacaggucaaccc

SEQ ID NO: 158 - DNA, 24 bp, Artificial Sequence
  synthetic oligonucleotide
gggttgacctgtctagcgttgatt

SEQ ID NO: 159 - RNA, 24 nt, Artificial Sequence
  synthetic oligonucleotide
ggguugaccugucuagcguugauu

SEQ ID NO: 160 - DNA, 57 bp, Artificial Sequence; identical to SEQ ID NO: 3
  synthetic oligonucleotide

SEQ ID NO: 161 - DNA, 19 bp, Artificial Sequence
  synthetic oligonucleotide
tcaacgctagacaggtcaa

SEQ ID NO: 162 - RNA, 19 nt, Artificial Sequence
  synthetic oligonucleotide
ucaacgcuagacaggucaa

SEQ ID NO: 163 - DNA, 19 bp, Artificial Sequence
  synthetic oligonucleotide
ttgacctgtctagcgttga

SEQ ID NO: 164 - RNA, 19 nt, Artificial Sequence
  synthetic oligonucleotide
uugaccugucuagcguuga

SEQ ID NO: 165 - DNA, 52 bp, Artificial Sequence
  synthetic oligonucleotide
tcaacgctagacaggtcaatttaaaaaaaaaaaaaaaaaaaaaaaaaaaaaa

SEQ ID NO: 166 - DNA, 19 bp, Artificial Sequence
  synthetic oligonucleotide
aatcaacgctagacaggtc

SEQ ID NO: 167 - RNA, 19 nt, Artificial Sequence
  synthetic oligonucleotide
aaucaacgcuagacagguc

SEQ ID NO: 168 - DNA, 19 bp, Artificial Sequence
  synthetic oligonucleotide
gacctgtctagcgttgatt

SEQ ID NO: 169 - RNA, 19 nt, Artificial Sequence
  synthetic oligonucleotide
gaccugucuagcguugauu

SEQ ID NO: 170 - DNA, 52 bp, Artificial Sequence
  synthetic oligonucleotide
aatcaacgctagacaggtctttaaaaaaaaaaaaaaaaaaaaaaaaaaaaaa

SEQ ID NO: 171 - DNA, 57 bp, Artificial Sequence
  synthetic oligonucleotide
aaucaacgcuagacaggucaaccctttaaaaaaaaaaaaaaaaaaaaaaaaaaaaaa

SEQ ID NO: 172 - DNA, 52 bp, Artificial Sequence
  synthetic oligonucleotide
ucaacgcuagacaggucaatttaaaaaaaaaaaaaaaaaaaaaaaaaaaaaa

SEQ ID NO: 173 - DNA, 1574 bp, Trichomonas vaginalis
tacttggttgatcctgccaaggaagcacacttaggtcatagattaagccatgcaagtgttagttcaggtaacgaaactgcgaatagctcattaatacgctcagaatctatttggcggcgaccaacaggtcttaaatggatagcagcagcaactctggtgctaatacatgcgattgtttctccagatgtgaattatggaggaaaagttgacctcatcagaggcacgccattcgactgagtgacctatcagcttgtacttagggtctttacctaggtaggctatcacgggtaacgggcggttaccgtcggactgccggagaaggcgcctgagagatagcgactatatccacgggtagcagcaggcgcgaaactttcccactcgagactttcggaggaggtaatgaccagttccattggtgccttttggtactgtggataggggtacggttttccaccgtaccgaaacctagcagagggccagtctggtgccagcagctgcggtaattccagctctgcgagtttgctccatattgttgcagttaaaacgccgtagtctgaattggccagcaatggtcgtacgtatttttacgttcactgtgaacaaatcaggacgcttagagtatggccacatgaatgactcagcgcagtatgaagtctttgttttcttccgaaaacaagctcaatgagagccatcgggggtagatctatctcatgacgagtggtggaatactttgactcatgagagagaagctgaggcgaaggcgtctacctagagggtttctgtcgatcaagggcgagagtaggagtatccaacaggattagagaccctggtagttcctaccttaaacgatgccgacaggagtttgtcatttgttaatggcagaatctttggagaaatcatagttcttgggctctgggggaactacgaccgcaaggctgaaacttgaaggaattgacggaagggcacaccaggggtggagcctgtggcttaatttgaatcaacacggggaaacttaccaggaccagatgttttttatgactgacaggcttcgggtctttcaggatattgtttttggtggtgcatggccgttggtggtgcgtgggttgacctgtctagcgttgattcagctaacgagcgagattatcgccaattatttactttgccgaagtccttcggttaaagttctaattgggactccctgcgattttagcaggtggaagagggtagcaataacaggtccgtgatgccctttagatgctctgggctgcacgcgtgctacaatgttaggatcaataggactgcgagcctgagagggtgcgctactcttataatccctaacgtagttgggattgacgtttgtaatcagcgtcatgaaccaggaatcctcgtaaatgtgtgtcaacaacgcacgttgaatacgtccctgccctttgtacacaccgcccgtcgctcctaccgattggatgactcggtgaaatcaccggatgcttacgagcagaaagtgattaaatcacgttatctagaggaaggagaagtcgtaacaaggtaacggtaggtgaacctgccgttggatc

SEQ ID NO: 174 - DNA, 550 bp, Artificial Sequence
  synthetic oligonucleotide
attgacggaagggcacaccaggggtggagcctgtggcttaatttgaatcaacacggggaaacttaccaggaccagatgttttttatgactgacaggcttcgggtctttcaggatattgtttttggtggtgcatggccgttggtggtgcgtgggttgacctgtctagcgttgattcagctaacgagcgagattatcgccaattatttactttgccgaagtccttcggttaaagttctaattgggactccctgcgattttagcaggtggaagagggtagcaataacaggtccgtgatgccctttagatgctctgggctgcacgcgtgctacaatgttaggatcaataggactgcgagcctgagagggtgcgctactcttataatccctaacgtagttgggattgacgtttgtaatcagcgtcatgaaccaggaatcctcgtaaatgtgtgtcaacaacgcacgttgaatacgtccctgccctttgtacacaccgcccgtcgctcctaccgattggatgactcggtgaaatcaccggatgcttacgagcagaa

SEQ ID NO: 175 - DNA, 330 bp, Artificial Sequence
  synthetic oligonucleotide
gcttcgggtctttcaggatattgtttttggtggtgcatggccgttggtggtgcgtgggttgacctgtctagcgttgattcagctaacgagcgagattatcgccaattatttactttgccgaagtccttcggttaaagttctaattgggactccctgcgattttagcaggtggaagagggtagcaataacaggtccgtgatgccctttagatgctctgggctgcacgcgtgctacaatgttaggatcaataggactgcgagcctgagagggtgcgctactcttataatccctaacgtagttgggattgacgtttgtaatcagcgtcatgaa

SEQ ID NO: 176 - DNA, 221 bp, Artificial Sequence
  synthetic oligonucleotide
ttactttgccgaagtccttcggttaaagttctaattgggactccctgcgattttagcaggtggaagagggtagcaataacaggtccgtgatgccctttagatgctctgggctgcacgcgtgctacaatgttaggatcaataggactgcgagcctgagagggtgcgctactcttataatccctaacgtagttgggattgacgtttgtaatcagcgtcatgaa

SEQ ID NO: 177 - DNA, 219 bp, Artificial Sequence
  synthetic oligonucleotide
ggcttcgggtctttcaggatattgtttttggtggtgcatggccgttggtggtgcgtgggttgacctgtctagcgttgattcagctaacgagcgagattatcgccaattatttactttgccgaagtccttcggttaaagttctaattgggactccctgcgattttagcaggtggaagagggtagcaataacaggtccgtgatgccctttagatgctctgg

SEQ ID NO: 178 - DNA, 132 bp, Artificial Sequence
  synthetic oligonucleotide
ctaattgggactccctgcgattttagcaggtggaagagggtagcaataacaggtccgtgatgccctttagatgctctgggctgcacgcgtgctacaatgttaggatcaataggactgcgagcctgagagggt

(19) United States
(12) Patent Application Publication
(10) Pub. No.: US 2022/0307093 A1
(43) Pub. Date: Sep. 29, 2022

(54) OLIGONUCLEOTIDES FOR USE IN DETERMINING THE PRESENCE OF TRICHOMONAS VAGINALIS IN A SAMPLE

(71) Applicant: Gen-Probe Incorporated, San Diego, CA (US)

(72) Inventors: Tamara Jane Angeles Johnson, San Diego, CA (US); Meghan Ann O'Donnell, San Diego, CA (US); Alice Jiang, San Diego, CA (US)

(21) Appl. No.: 17/596,786

(22) PCT Filed: Jul. 2, 2020
(86) PCT No.: PCT/US2020/040595

§ 371 Date: Dec. 17, 2021

Related U.S. Application Data
(60) Provisional application No. 62/870,308, filed on Jul. 3, 2019.

(51) Int. Cl. C12Q 1/6893; C12Q 1/6865

(52) U.S. Cl. CPC C12Q 1/6893; C12Q 1/6865

(57) ABSTRACT

Methods for use in multiplex amplification and or detection of Trichomonas vaginalis. The multiphase amplification provides fast, quantitative, sensitive detection with lower variability at low analyte concentrations. Described are detection probes, capture probes, amplification oligonucleotides, nucleic acid compositions, probe mixes, methods, and kits useful for amplifying and determining the presence of Trichomonas vaginalis in a test sample.

### CROSS-REFERENCE TO RELATED APPLICATIONS

This application claims benefit of priority under 35 U.S.C. § 119(e) to provisional application No. 62/870,308, filed Jul. 3, 2019 the contents of which is hereby incorporated by reference herein in its entirety.

### SEQUENCE LISTING

The Sequence Listing written in file DIA.0106.02_PCT_ST25 is 38 kilobytes in size, was created Jun. 25, 2020, and is hereby incorporated by reference.

### BACKGROUND

Trichomonas vaginalis is protozoan parasite that causes trichomoniasis, one of the most common and treatable of the sexually transmitted diseases. Worldwide, T. vaginalis infects approximately 180 million people per year, usually by direct person-to-person contact, making it the most common sexually transmitted disease (STD) agent. In the United States, it is believed that T. vaginalis infects an estimated 7 million people annually. Despite its prevalence there are no active control or prevention programs. Infections in women are known to cause vaginitis, urethritis, and cervicitis. Complications include premature labor, low-birth weight offspring, premature rupture of membranes, and post-abortion and post-hysterectomy infection. An association with pelvic inflammatory disease, tubal infertility, and cervical cancer have been reported. Trichomonas vaginalis has also been implicated as a co-factor in the transmission of HIV and other STD agents. The organism can also be passed to neonates during passage through the birth canal. In men, symptoms of trichomoniasis include urethral discharge, urethral stricture, epididymitis, the urge to urinate, and a burning sensation with urination. It is estimated 10-50% of T. vaginalis infections are asymptomatic in women. This number is likely higher in men.

Given its relative prevalence and association with other STDs, there is increasing interest in effectively diagnosing trichomoniasis. Cell culture is considered the current “gold standard” for clinical detection of T. vaginalis. Due to its relatively delicate nature, however, culturing the organism is technically challenging, and typically requires up to 7 days for maximum sensitivity. Even then, the sensitivity of cell culture methods is estimated to be only about 85-95%.

### SUMMARY

Described are oligonucleotides and compositions and methods of using the oligonucleotides and compositions for multi-phase (including dual-phase) amplification and/or detection of T. vaginalis. In some embodiments, oligonucleotides and compositions and methods of using the oligonucleotides and compositions are described for amplifying and/or detecting T. vaginalis in a sample. In multi-phase amplification, at least a portion of a target nucleic acid sequence is subjected to a first phase amplification reaction under conditions that do not support exponential amplification of the target nucleic acid sequence. The first phase amplification reaction generates a first amplification product, which is subsequently subjected to a second phase amplification reaction under conditions allowing exponential amplification of the first amplification product, thereby generating a second amplification product. Multi-phase amplification yields improved sensitivity and precision at the low end of analyte concentration compared with the single-phase format. Multi-phase amplification yields superior performance both in terms of precision and shorter detection time.

In some embodiments, multi-phase amplification of a T. vaginalis target nucleic acid sequence comprises:
a) contacting a sample containing or suspected of containing T. vaginalis target nucleic acid sequence with a target capture mixture, wherein the target capture mixture comprises a RNA polymerase promoter-containing oligonucleotide (promoter primer), and optionally a target capture oligonucleotide (TCO) to form a pre-amplification hybrid;
b) isolating the pre-amplification hybrid;
c) contacting the pre-amplification hybrid with a first phase amplification mixture; wherein the first phase amplification mixture comprises: a non-RNA polymerase promoter-containing oligonucleotide (non-promoter primer); a reverse transcriptase, an RNA polymerase, dNTPs, and NTPs, wherein the first phase amplification mixture is lacking in at least one component necessary for exponential amplification;
d) amplifying at least a portion of the target nucleic acid sequence of the pre-amplification hybrid in a substantially isothermal, transcription-associated amplification reaction under conditions that support linear amplification to from a first amplification product;
e) contacting the first amplification product with a second phase amplification mixture, wherein the second phase amplification mixture comprises the RNA polymerase promoter-containing oligonucleotide or the at least one component necessary for exponential amplification that is lacking in the first phase amplification mixture;
f) exponentially amplifying the first amplification product in a substantially isothermal transcription-associated amplification reaction to produce a second amplification product; and
g) detecting the second amplification product.

In some embodiments, the second phase amplification mixture contains a detection oligonucleotide.

In some embodiments, the T. vaginalis target nucleic acid sequence comprises a nucleotide sequence containing a portion the T. vaginalis 16S rRNA nucleotide sequence, represented by SEQ ID NO: 173, or a complement thereof.

In some embodiments, a target capture oligonucleotide (TCO) comprises: target specific (TS) sequence complementary to a region of the target nucleic acid sequence and an immobilized capture probe-binding region. The immobilized capture probe-binding region may be, but is not limited to a nucleic acid sequence. In some embodiments, the TCO comprises the nucleotide sequence of SEQ ID NO: 39, 40, or 41 or a complement thereof. In some embodiments, the TCO comprises the nucleotide sequence of SEQ ID NO: 1, 2, or 3, or a complement thereof.

In some embodiments, a promoter primer is an amplification oligonucleotide comprising: a 3′ target specific sequence and a 5′ promoter sequence comprising an RNA polymerase promoter sequence. The 3′ target specific sequence contains a region of complementarity to a region of the target nucleic acid (the promoter primer binding site) and hybridizes to the target nucleic acid. The promoter primer is capable of binding to its target sequence (promoter primer binding site) in the target nucleic acid and initiating template-dependent synthesis of RNA or DNA by an RNA- or DNA-dependent polymerase. The promoter sequence can be, but is not limited to, a T7 promoter sequence. In some embodiments, the promoter primer comprises the nucleotide sequence of SEQ ID NO: 42, 43, 44, 45, 46, 47, or 48. In some embodiments, the promoter primer comprises SEQ ID NO: 4, 5, 6, 7, 8, 9, 10, 11, or 12.

In some embodiments, the pre-amplification hybrid comprises the target nucleic acid hybridized the promoter primer. In some embodiments, the pre-amplification hybrid comprises the target nucleic acid hybridized to each of the TCO and promoter primer. In some embodiments, isolating the pre-amplification hybrid comprises capturing the pre-amplification hybrid using a solid support. In some embodiments, the solid support includes an immobilized capture probe. The solid support can be, but is not limited to, magnetically attractable particles. In some embodiments, isolating the pre-amplification hybrid comprises removing promoter primer that is not hybridized to the target nucleic acid.

In some embodiments, a non-promoter primer (also termed NT7 primer) is an amplification oligonucleotide that binds specifically to its target sequence in a cDNA product of extension of the promoter primer, downstream from the promoter-primer end. The promoter primer is combined with non-promoter primer to form an amplification pair and together are configured to amplify a portion of the target nucleic acid. The non-promoter primer lacks the RNA polymerase promoter sequence of the promoter primer. In some embodiments, the non-promoter primer comprises the nucleotide sequence of SEQ ID NO: 49, 50, 51, 52, 53, 54, or 55. In some embodiments, the non-promoter primer comprises the nucleotide sequence of SEQ ID NO: 13, 14, 15, 16, 17, 18, or 19.

In some embodiments, during the first phase isothermal transcription-associated amplification reaction, the promoter primer, bound specifically to the target nucleic acid at its target sequence, is extended by reverse transcriptase (RT) to create a cDNA copy, using the target nucleic acid as a template. The non-promoter primer is then enzymatically extended to produce a double strand DNA, using the cDNA as template. Next, the double strand DNA serves as template for RNA transcription from the RNA polymerase promoter provided by the promoter primer. The non-promoter primer then binds to the RNA and is extended by reverse transcriptase to yield the first amplification product. In the absence of additional promoter primer, exponential amplification does not occur. The first amplification product is then contacted with the second phase amplification mixture to initiate the exponential second phase amplification.

In some embodiments, each of the first and second phase isothermal transcription-associated amplification reactions include an RNA polymerase and a reverse transcriptase. In some embodiments, the reverse transcriptase includes an endogenous RNase H activity.

In some embodiments, a detection oligonucleotide contains a target specific (TS) sequence complementary to a nucleobase sequence present in the second amplification product. The detection oligonucleotide target specific sequence is 10 or more nucleobases in length. In some embodiments, the detection oligonucleotide target specific sequence is 10-30 nucleobases in length. In some embodiments, the detection oligonucleotide contains a detectable molecule. In some embodiments, the detectable molecule comprises a fluorophore. In some embodiments, the detection oligonucleotide contains a fluorophore and a quencher. A detection oligonucleotide can be, but is not limited to, a Torch. The detection oligo can be DNA, RNA, or a combination of DNA and RNA. The detection oligonucleotide can also have one or more modified nucleotides, including, but not limited to, methoxy RNA. In some embodiments, the Torch comprises the nucleotide sequence of SEQ ID NO: 56, 57, 58, 59, 60, 61, or 62. In some embodiments, the Torch comprises the nucleotide sequence of SEQ ID NO: 20, 21, 22, 23, 24, 25, 26, 27, or 28.

In some embodiments, compositions suitable for use in a first phase amplification of a multi-phase amplification of T. vaginalis comprise: (a) an optional target capture oligonucleotide, (b) a promoter primer hybridized to a first portion of a T. vaginalis target nucleic acid sequence; (c) a non-promoter primer; and (d) additional components necessary to amplify the target nucleic acid during a linear first phase amplification reaction, but lacking at least one component required for exponential amplification of the target nucleic acid sequence. In some embodiments, the lacking at least one component necessary for exponential amplification is additional (free) promoter primer. In some embodiments, the first phase amplification lacks promoter primer that is not hybridized to the target nucleic acid. The additional components can include one or more of: RNA-dependent DNA polymerase, RNA polymers, dNTPs, NTPs, buffers, and salts.

In some embodiments, compositions suitable for use in a second or subsequent phase amplification of a multi-phase amplification of T. vaginalis comprise: (a) a first amplification product, (b) promoter primer, (c) non-promoter primer, (d) other necessary components necessary to amplify the target nucleic acid during an exponential second phase amplification reaction. The additional components can include one or more of: RNA-dependent DNA polymerase, RNA polymers, dNTPs, NTPs, buffers, and salts.

In some embodiments, methods are described for multi-phase amplification and/or detection of T. vaginalis. The methods comprise:
(a) contacting a sample containing or suspected of containing a T. vaginalis target nucleic acid with a promoter primer specific for a first portion of the target nucleic acid sequence, under conditions allowing hybridization of the promoter primer to the first portion of the target nucleic acid sequence, thereby generating a pre-amplification hybrid that includes the first amplification oligonucleotide and the target nucleic acid sequence;
(b) isolating the pre-amplification hybrid by target capture onto a solid support followed by washing to remove any of the promoter primer that did not hybridize to the first portion of the target nucleic acid sequence in step (a);
(c) amplifying, in a first phase amplification reaction mixture, at least a portion of the target nucleic acid sequence of the pre-amplification hybrid isolated in step (b) in a first phase, substantially isothermal, transcription-associated amplification reaction under conditions that support linear amplification thereof, but do not support exponential amplification thereof (i.e., the first phase amplification reaction mixture lacks at least one component necessary for exponential amplification of the first amplification product), thereby resulting in a reaction mixture including a first amplification product;
(d) combining the reaction mixture including the first amplification product with the at least one component necessary for exponential amplification of the first amplification product, but that is lacking from the reaction mixture that includes the first amplification product, to produce a second phase amplification reaction mixture;
(e) exponentially amplifying the first amplification product in a second phase amplification mixture, in a substantially isothermal transcription-associated amplification reaction, to produce a second amplification product; and
(f) optionally detecting the second amplification product.

In some embodiments, the at least one component necessary for exponential amplification of the first amplification product includes the primer promoter (e.g., promoter primer in addition to promoter primer hybridized with the target nucleic acid and isolated as part of the pre-amplification hybrid). In some embodiments, the first amplification product of step (c) is a cDNA molecule with the same polarity as the target nucleic acid sequence in the sample, and the second amplification product of step (e) is an RNA molecule. The second amplification product can be detected using a sequence-specific detection probe. The sequence-specific detection probe can be, but is not limited to, a conformation-sensitive probe that produces a detectable signal when hybridized to the second amplification product. In some embodiments, the sequence-specific detection probe in step is a fluorescently labeled sequence-specific hybridization probe. Detecting can be performed at regular time intervals. In some embodiments, the detecting is performed in real time. In some embodiments, detecting the second amplification product comprises quantifying the target nucleic acid sequence in the sample using a linear calibration curve.

In some embodiments, the described oligonucleotides, compositions, and methods can be used to detect T. vaginalis 16SrRNA present in a sample at less than or equal to 10 cells/ml, less than or equal to 1 cell/ml, less than or equal to 0.1 cell/ml, or less than or equal to 0.01 cells/ml copies. In some embodiments, the described oligonucleotides, compositions, and methods can be used to detect T. vaginalis 16S rRNA in a sample having at 0.002 or more cells/ml. In some embodiments, the detection rate, using the described oligonucleotides is greater than or equal to 90% or greater than to equal to 95% when the T. vaginalis is present at 0.002 or more cells/ml in a sample.

In some embodiments, the described oligonucleotides, compositions, and methods are suitable for use in amplifying and/or detecting T. vaginalis in multiplex multi-phase reactions. The multiplex multi-phase reactions can be used to detect T. vaginalis and one or more other target sequences and/or organisms. In some embodiments, CV/TV multiplex assays are described. The CV/TV multiplex assay contains oligonucleotides for the capture, amplification and detection of C. albicans, C. tropicalis, C. dubliniensis, C. parapsilosis, C. glabrata, and T. vaginalis.

### BRIEF DESCRIPTION OF THE DRAWING

FIG. 1 Flow diagram illustrating multi-phase (including dual-phase) forward Transcription-Mediated Amplification (TMA). In this embodiment, an amplification primer containing a T7 promoter (“T7 primer”) hybridizes to a target nucleic acid sequence during target capture, followed by removal of excess T7 primer. The amplification process is divided into at least two distinct phases. During the first phase, a NT7 primer is introduced along with all of the requisite amplification and enzyme reagents (AR and ER, respectively), with the exception of additional T7 primer (RT: reverse transcriptase; T7: T7 RNA polymerase). In the presence of reverse transcriptase, the T7 primer hybridized to the target is extended, creating a cDNA copy, and the target RNA template is degraded by RNase H activity of RT. The NT7 primer subsequently hybridizes to the cDNA and is then extended, filling in the promoter region of the T7 primer and creating an active, double-stranded template. The T7 polymerase then produces multiple RNA transcripts from the template. The NT7 primer subsequently hybridizes to the RNA transcripts and is extended, producing promoterless cDNA copies of the target RNA template. The RNA strands are then degraded by RNase activity of RT. Because no additional T7 primer is available in the phase 1 amplification mixture, the reaction cannot proceed further. The second phase is then started with the addition of T7 primer, thus initiating exponential amplification of the cDNA pool produced in phase 1.

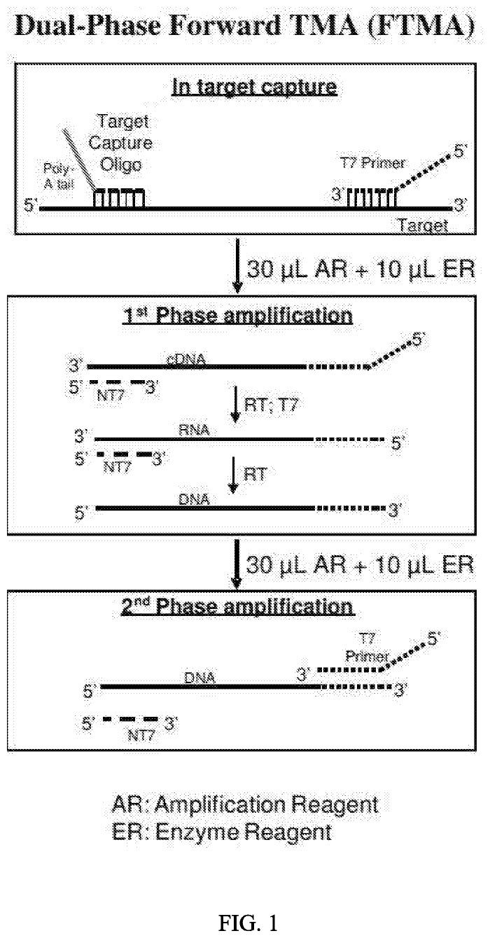

### DETAILED DESCRIPTION

For clarity of disclosure, and not by way of limitation, the detailed description of the invention is divided into the subsections that follow.

A. Definitions

All patents, applications, published applications and other publications referred to herein are incorporated by reference in their entireties. To the extent different content might be associated with the same citation at different times, content associated with the citation at the effective filing date is meant. The effective filing date means the earliest priority date at which the citation is disclosed. Unless otherwise apparent from the context any element, embodiment, step, feature or aspect of the invention can be performed in combination with any other If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

Approximating language, throughout the specification and claims, may be applied to modify any quantitative or qualitative representation that could permissibly vary without resulting in a change in the basic function to which it is related. Accordingly, a value modified by a term such as “about” or “approximately” is not to be limited to the precise value specified, and may include values that differ from the specified value. In some embodiments, about or approximately indicates insignificant variation and/or variation of less than 5%.

A “sample” is a specimen or substance that contains or is suspected of containing an analyte of interest, e.g., microbe, virus, nucleic acid such as a gene (e.g., target nucleic acid), or components thereof, which includes nucleic acid sequences in or derived from an analyte. Samples may be from any source, such as, but not limited to, biological specimens, clinical specimens, and environmental sources. Biological specimens include, but are not limited to, tissue or material derived from a living or dead organism that may contain an analyte or nucleic acid in or derived from an analyte. Examples of biological samples include, but are not limited to, respiratory tissue, exudates (e.g., bronchoalveolar lavage), biopsy, sputum, tracheal aspirates, saliva, mucus, peripheral blood, plasma, serum, lymph node, cerebrospinal fluid, gastrointestinal tissue, feces, urine, genitourinary, biological fluids, tissues or materials, and biopsies, including, but not limited to, specimens from or derived from genital lesions, anogenital lesions, oral lesions, mucocutaneous lesions, skin lesions, ocular lesions or combinations thereof. Examples of environmental samples include, but are not limited to, water, ice, soil, slurries, debris, biofilms, airborne particles, and aerosols. Samples may also include samples of in vitro cell culture constituents including, e.g., conditioned media resulting from the growth of cells and tissues in culture medium. Samples may be processed specimens or materials, such as obtained from treating a sample by using filtration, centrifugation, sedimentation, or adherence to a medium, such as matrix or support. Other processing of samples may include, but are not limited to, treatments to physically or mechanically disrupt tissue, cellular aggregates, or cells to release intracellular components that include nucleic acids into a solution which may contain other components, such as, but not limited to, enzymes, buffers, salts, detergents and the like.

The term “contacting” means bringing two or more components together. Contacting can be achieved by mixing all the components in a fluid or semi-fluid mixture. Contacting can also be achieved when one or more components are brought into physical contact with one or more other components on a solid surface such as a solid tissue section or a substrate.

“Nucleic acid” refers to a polynucleotide compound, which includes oligonucleotides, comprising nucleosides or nucleoside analogs that have nitrogenous heterocyclic bases or base analogs, covalently linked by standard phosphodiester bonds or other linkages. Nucleic acids include RNA, DNA, chimeric DNA-RNA polymers or analogs thereof. In a nucleic acid, the backbone may be made up of a variety of linkages, including, but not limited to, one or more of sugar-phosphodiester linkages, peptide-nucleic acid (PNA) linkages (PCT Pub No. WO 95/32305), phosphorothioate linkages, methylphosphonate linkages, or combinations thereof. Sugar moieties in a nucleic acid may be, but are not limited to, ribose, deoxyribose, or similar compounds with substitutions, e.g., 2′ methoxy and 2′ halide (e.g., 2′-F) substitutions. Nitrogenous bases may be, but are not limited to, conventional bases (A, G, C, T, U), analogs thereof (e.g., inosine; The Biochemistry of the Nucleic Acids 5-36, Adams et al., ed., 11th ed., 1992), derivatives of purine or pyrimidine bases (e.g., N4-methyl deoxyguanosine, deaza- or aza-purines, deaza- or aza-pyrimidines, pyrimidines or purines with altered or replacement substituent groups at any of a variety of chemical positions, e.g., 2-amino-6-methylaminopurine, O6-methylguanine, 4-thio-pyrimidines, 4-amino-pyrimidines, 4-dimethylhydrazine-pyrimidines, and O4-alkyl-pyrimidines, or pyrazolo-compounds, such as unsubstituted or 3-substituted pyrazolo[3,4-d]pyrimidine (e.g., U.S. Pat. Nos. 5,378,825, 6,949,367 and PCT Pub. No. WO 93/13121)). Nucleic acids may include “abasic” positions in which the backbone does not have a nitrogenous base at one or more locations (U.S. Pat. No. 5,585,481), e.g., one or more abasic positions may form a linker region that joins separate oligonucleotide sequences together. A nucleic acid may comprise only conventional sugars, bases, and linkages as found in conventional RNA and DNA, or may include conventional components and substitutions (e.g., conventional bases linked by a 2′ methoxy backbone, or a polymer containing a mixture of conventional bases and one or more analogs). The term includes “locked nucleic acids” (LNA), which contain one or more LNA nucleotide monomers with a bicyclic furanose unit locked in a RNA mimicking sugar conformation, which enhances hybridization affinity for complementary sequences in ssRNA, ssDNA, or dsDNA (Vester et al., 2004, Biochemistry 43(42):13233-41). Nucleic acids may include modified bases. Modified bases may alter the function or behavior of the nucleic acid. References, particularly in the claims, to “the sequence of SEQ ID NO: X” refer to the base sequence of the corresponding sequence listing entry and do not require identity of the backbone (e.g., RNA, 2′-O-Me RNA, or DNA) or base modifications (e.g., methylation of cytosine residues) unless otherwise indicated.

A “target nucleic acid” or “target” is a nucleic acid containing a target nucleic acid sequence. A “target nucleic acid sequence,” “target sequence” or “target region” is a specific deoxyribonucleotide or ribonucleotide sequence comprising a nucleotide sequence of a target organism, such as T. vaginalis, to be amplified. A target sequence, or a complement thereof, contains sequences that hybridize to capture oligonucleotides, amplification oligonucleotides, and/or detection oligonucleotides used to amplify and/or detect the target nucleic acid. The target nucleic acid may include other sequences besides the target sequence which may not be amplified. Target nucleic acids may be DNA or RNA and may be either single-stranded or double-stranded A target nucleic acid can be, but is not limited to, a genomic nucleic acid, a transcribed nucleic acid, such as an rRNA, or a nucleic acid derived from a genomic or transcribed nucleic acid.

An “oligonucleotide,” “oligomer,” or “oligo” is a polymer made up of two or more nucleoside subunits or nucleobase subunits coupled together. The oligonucleotide may be DNA and/or RNA and analogs thereof. In some embodiments, the oligonucleotides are in a size range having a 5 to 15 nt lower limit and a 50 to 500 nt upper limit. In some embodiments, the oligonucleotides are in a size range of 10-100 nt, 10-90 nt, 10-80 nt. 10-70 nt, or 10-60 nt. An oligonucleotide does not consist of wild-type chromosomal DNA or the in vivo transcription products thereof. Oligonucleotides can made synthetically by using any well-known in vitro chemical or enzymatic method, and may be purified after synthesis by using standard methods, e.g., high-performance liquid chromatography (HPLC). Described are oligonucleotides include RNA polymerase promoter-containing oligonucleotides (also termed promoter primer; e.g., T7 primers), non-RNA polymerase promoter-containing oligonucleotides (e.g., NT7 primers, also termed non-promoter primers), detection probe oligonucleotides (also termed detection oligo or detection probe; e.g., Torches), and target capture oligonucleotides (TC oligos). The N7 and NT7 primers are priming oligonucleotides and can be referred to as “amplification oligonucleotides.”

The sugar groups of the nucleoside subunits may be ribose, deoxyribose and analogs thereof, including, for example, ribonucleosides having a 2′-substitution, including, but not limited to, e.g., methoxy RNA. (Oligonucleotides including nucleoside subunits having 2′ substitutions and which are useful as detection probes, capture probes, and/or amplification oligonucleotides are disclosed by Becker et al., “Method for Amplifying Target Nucleic Acids Using Modified Primers,” U.S. Pat. No. 6,130,038.) The nucleoside subunits may be joined by linkages such as phosphodiester linkages, modified linkages, or by non-nucleotide moieties which do not prevent hybridization of the oligonucleotide to its complementary target nucleic acid sequence. Modified linkages include those linkages in which a standard phosphodiester linkage is replaced with a different linkage, such as a phosphorothioate linkage or a methylphosphonate linkage. The nucleobase subunits may be joined, for example, by replacing the natural deoxyribose phosphate backbone of DNA with a pseudo-peptide backbone, such as a 2-aminoethylglycine backbone which couples the nucleobase subunits by means of a carboxymethyl linker to the central secondary amine. (DNA analogs having a pseudo-peptide backbone are commonly referred to as “peptide nucleic acids” or “PNA”, and are disclosed by Nielsen et al., “Peptide Nucleic Acids,” U.S. Pat. No. 5,539,082.) Other non-limiting examples of oligonucleotides or oligomers. Any nucleic acid analog is contemplated by the present disclosure, provided that the modified oligonucleotide can hybridize to a target nucleic acid under stringent hybridization conditions or amplification conditions. In the case of detection probes, the modified oligonucleotides must also be capable of preferentially hybridizing to the target nucleic acid under stringent hybridization conditions. The described oligonucleotides are configured to hybridize specifically to T. vaginalis or Candida target nucleic acids or nucleic acid sequences derived from T. vaginalis or Candida target nucleic acids.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “complementarity” refers to the ability of a polynucleotide to form hydrogen bond(s) (hybridize) with another polynucleotide sequence by either traditional Watson-Crick or other non-traditional types. A percent complementarity indicates the percentage of bases, in a contiguous strand, in a first nucleic acid sequence which can form hydrogen bonds (e.g., Watson-Crick base pairing) with a second nucleic acid sequence (e. g., 5, 6, 7, 8, 9, 10 out of 10 being 50%, 60%, 70%, 80%, 90%, and 100% complementary). Percent complementarity is calculated in a similar manner to percent identify.

By “stringent hybridization conditions” or “stringent conditions” is meant conditions permitting an oligonucleotide to preferentially hybridize to a target nucleic acid (for example, rRNA or rDNA derived from T. vaginalis) and not to nucleic acid derived from a closely related non-target microorganism. Stringent hybridization conditions may vary depending upon factors including the GC content and length of the probe, the degree of similarity between the probe sequence and sequences of non-target sequences which may be present in the test sample, and the target sequence. Hybridization conditions include the temperature and the composition of the hybridization reagents or solutions.

“Amplification” of a target nucleic acid refers to the process of creating, in vitro, multiple copies of a target nucleic acid that are identical and/or complementary to at least a portion of a target nucleic acid sequence. An example of a nucleic acid amplification procedure include transcription transcription-mediated amplification (TMA, U.S. Pat. Nos. 5,399,491, 5,554,516, 5,437,990, 5,130,238, 4,868,105, and 5,124,246, incorporated herein by reference).

“Single phase amplification” refers for nucleic amplification reactions in which all components required for nucleic acid amplification are present in the reaction mixture when amplification is started. In single phase amplifications, undesired side reactions that are initiated along with the desired amplification reaction often compete with and degrade overall performance of the desired amplification reaction. In multiplex single phase amplification reactions, amplification of analytes that are present at higher amounts in the reaction mixture or analytes whose overall amplification efficiency is higher than that of other analytes unduly compete with and degrade amplification of the other analytes in the mixture.

An “amplification product” is a nucleic acid molecule generated in a nucleic acid amplification reaction and which is derived from a target nucleic acid or a nucleic acid itself derived from the target nucleic acid. An amplification product contains all or a portion of a target nucleic acid sequence that may be of the same or opposite sense as the target nucleic acid.

“Linear amplification” refers to an amplification mechanism that is designed to produce an increase in the target nucleic acid linearly proportional to the amount of target nucleic acid in the reaction. For instance, multiple RNA copies can be made from a DNA target using a transcription-associated reaction, where the increase in the number of copies can be described by a linear factor (e.g., starting copies of template×n). In some embodiments, a first phase linear amplification in a multiphase amplification procedure increases the starting number of target nucleic acid strands or the complements thereof by at least 10 fold, at least 100 fold, or at least 1,000 fold before the second phase amplification reaction is initiated. An example of a linear amplification system is “T7-based Linear Amplification of DNA” (TLAD; see Liu et al., BMC Genomics, 4: Art. No. 19, May 9, 2003). Other methods are disclosed herein. Accordingly, the term “linear amplification” refers to an amplification reaction which does not result in the exponential amplification of a target nucleic acid sequence. The term “linear amplification” does not refer to a method that simply makes a single copy of a nucleic acid strand, such as the transcription of an RNA molecule into a single cDNA molecule as in the case of reverse transcription (RT)-PCR.

“Exponential amplification” refers to nucleic acid amplification that is designed to produce an increase in the target nucleic acid geometrically proportional to the amount of target nucleic acid in the reaction. For example, PCR produces one DNA strand for every original target strand and for every synthesized strand present. Similarly, transcription-associated amplification produces multiple RNA transcripts for every original target strand and for every subsequently synthesized strand. The amplification is exponential because the synthesized strands are used as templates in subsequent rounds of amplification. An amplification reaction need not actually produce exponentially increasing amounts of nucleic acid to be considered exponential amplification, so long as the amplification reaction is designed to produce such increases.

The term “substantially isothermal amplification” refers to an amplification reaction that is conducted at a substantially constant temperature. The isothermal portion of the reaction may be preceded or followed by one or more steps at a variable temperature, for example, a first denaturation step and a final heat inactivation step or cooling step. It will be understood that this definition does not exclude small variations in temperature but is rather used to differentiate the isothermal amplification techniques from other amplification techniques known in the art that basically rely on “cycling temperatures” in order to generate the amplified products. Isothermal amplification differs from PCR, for example, in that the latter relies on cycles of denaturation by heating followed by primer hybridization and polymerization at a lower temperature.

Reference to a range of value also includes integers within the range and subranges defined by integers in the range.

B. Methods of Multiphase Amplification

The disclosed methods use aspects of isothermal amplification systems that are generally referred to as “transcription-associated amplification” methods, which amplify a target sequence by producing multiple transcripts from a nucleic acid template. Such methods generally use one or more amplification oligonucleotides, of which one provides an RNA polymerase promoter sequence, deoxyribonucleoside triphosphates (dNTPs), ribonucleoside triphosphates (NTPs), and enzymes with RNA polymerase and DNA polymerase activities to generate a functional promoter sequence near the target sequence and then transcribe the target sequence from the promoter (e.g., U.S. Pat. Nos. 4,868,105, 5,124,246, 5,130,238, 5,399,491, 5,437,990, 5,554,516 and 7,374,885; and PCT Pub. Nos. WO 1988/001302, WO 1988/010315 and WO 1995/003430). Examples include Transcription-Mediated Amplification (TMA), nucleic acid sequence based amplification (NASBA) and Self-Sustained Sequence Replication (3SR).

To aid in understanding of some of the embodiments disclosed herein, the TMA method that has been described in detail previously (e.g., U.S. Pat. Nos. 5,399,491, 5,554,516 and 5,824,518) is briefly summarized. In TMA, a target nucleic acid that contains the sequence to be amplified is provided as single stranded nucleic acid (e.g., ssRNA or ssDNA). Any conventional method of converting a double stranded nucleic acid (e.g., dsDNA) to a single-stranded nucleic acid may be used. A promoter primer (e.g., T7 primer) binds specifically to the target nucleic acid at its target sequence and a reverse transcriptase (RT) extends the 3′ end of the promoter primer using the target strand as a template to create a cDNA copy, resulting in a RNA:cDNA duplex. RNase activity (e.g., RNase H of RT enzyme) digests the RNA of the RNA:cDNA duplex. A second primer (e.g., NT7 primer) binds specifically to its target sequence in the cDNA, downstream from the promoter-primer end. Then RT synthesizes a new DNA strand by extending the 3′ end of the second primer using the cDNA as a template to create a dsDNA that contains a functional promoter sequence. RNA polymerase specific for the functional promoter initiates transcription to produce multiple (e.g., 100 to 1000) RNA transcripts (amplified copies or amplicons) complementary to the initial target strand. The second primer binds specifically to its target sequence in each amplicon and RT creates a cDNA from the amplicon RNA template to produce a RNA:cDNA duplex. RNase digests the amplicon RNA from the RNA:cDNA duplex and the target specific sequence of the promoter primer binds to its complementary sequence in the newly synthesized DNA and RT extends the 3′ end of the promoter primer as well as the 3′ end of the cDNA to create a dsDNA that contains a functional promoter to which the RNA polymerase binds and transcribes additional amplicons that are complementary to the target strand. Autocatalytic cycles that use these steps repeatedly during the reaction produce amplification of the initial target sequence. Amplicons may be detected during amplification (real-time detection) or at an end point of the reaction (end-point detection) by using a probe that binds specifically to a sequence contained in the amplicons. Detection of a signal resulting from the bound probes indicates the presence of the target nucleic acid in the sample.

Described are methods of amplifying and/or detecting Trichomonas vaginalis using a multiphase amplification procedure. The methods comprise amplifying T. vaginalis target nucleic acid sequence in a sample including the following steps. Initially, the target nucleic acid sequence is subjected to a first phase amplification reaction under conditions that do not support exponential amplification of the target nucleic acid sequence. The first phase amplification reaction generates a first amplification product, which is subsequently subjected to a second phase amplification reaction under conditions allowing exponential amplification of the first amplification product, thereby generating a second amplification product.

The T. vaginalis target nucleic acid sequence may be any RNA or DNA sequence. In some embodiments, the target sequence is an RNA sequence, such as an mRNA or rRNA sequence. In some embodiments, the T. vaginalis target nucleic acid sequence is a 16S rRNA sequence represented by SEQ ID NO: 173 or a complement thereof. In some embodiments, the T. vaginalis target nucleic acid sequence comprises or consists of SEQ ID NO: 174 or a complement thereof. In some embodiments, the T. vaginalis target nucleic acid sequence comprises or consists of SEQ ID NO: 175 or a complement thereof. In some embodiments, the T. vaginalis target nucleic acid sequence consists of a nucleotide sequence present in SEQ ID NO: 173, 174, or 175 or a complement thereof.

In some embodiments, the portion of the target sequence targeted by the promoter primer (promoter primer binding site) may be different (e.g. non-overlapping) from the portion targeted by the target capture oligonucleotide (if used). A promoter primer binding site may fully or partially overlap with, or be identical to, the target capture oligonucleotide binding site. In some embodiments, the amplified region of the target sequence partially or completely overlaps the target capture binding site. In some embodiments, the amplified region of the target sequence does not overlap the target capture binding site.

In some embodiments, before the first amplification step, the sample is contacted with one or more promoter primers under conditions allowing hybridization of the promoter primer to a portion of the target nucleic acid sequence in the sample. A promoter primer comprises a 3′ target specific (TS) sequence, an RNA polymerase promoter sequence, and optionally, one or more tag sequences. The RNA polymerase promoter sequence is recognized by an RNA polymerase, such as T7 RNA polymerase. A tag sequence can be, but is not limited to, an amplification primer binding site, a specific binding site used for capture, or a sequencing primer binding site. The one or more promoter primers can target the same or different target nucleic acid sequences. The different target nucleic acid sequence can be from the same or different organisms.

In some embodiments, it may be desirable to isolate the target nucleic acid sequence prior to the first phase amplification. To this end, the sample may be contacted with a target capture oligonucleotide under conditions allowing hybridization of the target capture oligonucleotide to a portion of the target nucleic acid sequence (TCO binding site). In some embodiments, the target nucleic acid is captured onto a solid support directly, for example by interaction with an immobilized capture probe. In some embodiments, the target nucleic acid is captured onto the solid support as a member of a three molecule complex (pre-amplification hybrid), with the target capture oligonucleotide bridging the target nucleic acid and the immobilized capture probe. In some embodiments, the solid support comprises a plurality of magnetic or magnetizable particles or beads that can be manipulated using a magnetic field. The step of isolating the target nucleic acid sequence can include washing the target capture oligonucleotide:target nucleic acid sequence hybrid to remove undesired components that may interfere with subsequent amplification. The step of isolating the target nucleic acid sequence can also include washing the target capture oligonucleotide:target nucleic acid sequence hybrid to substantially remove excess promoter primer that is not hybridized to the target nucleic acid.

In some embodiments, the step of isolating the target nucleic acid sequence includes contacting the sample with a promoter primer and a TCO under conditions allowing hybridization of the promoter primer and TCO to the target nucleic acid sequence. The portion of the target sequence targeted by the promoter primer may be different (e.g. non-overlapping) from the portion targeted by the target capture oligonucleotide. The portion of the target sequence targeted by the promoter primer may fully or partially overlaps with, or even be identical to, the portion targeted by the target capture oligonucleotide. The promoter primer comprises a 3′ target specific sequence, an RNA polymerase promoter sequence, and optionally, one or more tag sequences. In some embodiments, the RNA polymerase promoter sequence is recognized by an RNA polymerase, such as T7 RNA polymerase. A tag sequence can be, but is not limited to, an amplification primer binding site, a specific binding site used for capture, or a sequencing primer binding site.

In some embodiments, one or more target capture oligonucleotides and one or more promoter primers are provided in a target capture reagent (TCR mixture). The one or more promoter primers can be hybridized to one or more target nucleic acid sequences to form pre-amplification hybrids (along with the TCO(s)) and isolated along with the one or more target nucleic acid sequences during the target capture step. One advantage of this method is that by hybridizing the promoter primer(s) to the target nucleic acid sequence(s) during target capture, the captured nucleic acids can be washed to remove sample components, including unhybridized promoter primers. In a multiphase reaction, removing unhybridized promoter primers allows the first phase amplification to occur without interference from the excess promoter primers, thereby substantially reducing or eliminating the problems common to multiplex reactions. In single phase multiplex amplification reactions, the primers can interfere with one another. Excess primers more readily misprime (hybridize to non-target nucleic acids) in uniplex and in multiplex reactions. In a multiplex reaction where the various organisms each have their own rRNA and oligonucleotides, mispriming is a bigger concern. Multiphase amplification addresses these problems by hybridizing the promoter primer to its intended target under stringent conditions, then washing away the excess promoter primer. The resulting 1:1 primer/target ratio present in the first phase amplification reaction of a multiphase amplification can boost the population of target nucleic acids to a level that allows for the subsequence addition of excess primer while reducing the level of mispriming or the effects of any mispriming on amplification.

The first phase amplification reaction is carried out under conditions that do not support exponential amplification of the target nucleic acid sequence. In some embodiments, the first phase amplification reaction is a linear amplification reaction. The first phase amplification reaction will typically produce from about 2-fold to about 10,000-fold amplification. In some embodiments, the first phase amplification reaction will produce about 10-fold to about 10,000-fold amplification of the target nucleic acid sequence. In some embodiments, the first phase amplification reaction is substantially isothermal, i.e., it does not involve thermal cycling characteristic of PCR and other popular amplification techniques. The first phase amplification reaction can be performed at 43±2° C., 43±2° C., 42±1° C., 42±0.5° C., 43±0.5° C., 44±0.5° C., 41-45° C., or 42-44° C.

In some embodiments, the first phase amplification reaction involves contacting the target nucleic acid sequence with a first phase amplification reaction mixture (e.g., AMP mixture) that supports linear amplification of the target nucleic acid sequence and lacks at least one component that is required for its exponential amplification. In some embodiments, at least one component that is required for its exponential amplification is additional or excess promoter primer. In some embodiments, the AMP reaction mixture comprises one or more amplification enzymes. The one or more amplification enzymes can be, but are not limited to: a DNA polymerase, an RNA polymerase, or a combination thereof. The DNA polymerase can be, but is not limited to, an RNA-dependent DNA polymerase (reverse transcriptase), a DNA-dependent DNA polymerase, or a combination thereof. In some embodiments, the AMP mixture comprises a ribonuclease (RNase), such as an RNase H or a reverse transcriptase with an RNase H activity. In some embodiments, the AMP mixture includes a reverse transcriptase with an RNase H activity and an RNA polymerase. The RNA polymerase can be, but is not limited to, a T7 RNA polymerase. In some embodiments, the AMP mixture contains one or more non-RNA polymerase promoter-containing amplification oligonucleotides (e.g., non-promoter primers (i.e., NT7 primers)). The one or more non-promoter primers can target the same or different target nucleic acid sequences. The different target nucleic acid sequence can be from the same or different organisms. In some embodiments, the AMP mixture comprises: one or more non-promoter primer(s), an RNA polymerase, ribonucleotide triphosphates (NTPs), and deoxyribonucleotide triphosphates (dNTPs). The AMP mixture may additionally contain other components, including, but not limited to, buffers, dNTPs, NTPs, and salts.

In some embodiments, the first phase amplification reaction is unable to support an exponential amplification reaction because one or more components required for exponential amplification are lacking, an agent is present which inhibits exponential amplification, and/or the temperature of the reaction mixture is not conducive to exponential amplification. Without limitation, the lacking one or more components required for exponential amplification and/or inhibitor and/or reaction condition can be selected from any of: an amplification oligonucleotide (e.g., a promoter primer, a non-promoter primer, or a combination thereof), an enzyme (e.g., a polymerase, such as an RNA polymerase), a nuclease (e.g., an exonuclease, an endonuclease, a cleavase, an RNase, a phosphorylase, a glycosylase, etc.), an enzyme co-factor, a chelator (e.g., EDTA or EGTA), ribonucleotide triphosphates (NTPs), deoxyribonucleotide triphosphates (dNTPs), Mg²⁺, a salt, a buffer, an enzyme inhibitor, a blocking oligonucleotide, pH, temperature, salt concentration, and any combination thereof. In some cases, the lacking component may be involved indirectly, such as an agent that reverses the effects of an inhibitor of exponential amplification which is present in the first phase reaction. In some embodiments, the lacking one or more components is a promoter primer (additional promoter primer in excess of the promoter primer hybridized to the target nucleic acid as part of the pre-amplification hybrid).

The second phase (or later phase, if there are more than 2 phases) amplification reaction is carried out under conditions that allow exponential amplification of the target nucleic acid sequence. In some embodiments, the second phase amplification reaction is an exponential amplification reaction. In some embodiments, the second phase amplification reaction is a substantially isothermal reaction, such as, for example, a transcription-associated amplification reaction or a strand displacement amplification reaction. In some embodiments, the second phase amplification reaction is a Transcription-Mediated Amplification (TMA) reaction. In some embodiments, the second phase amplification reaction is performed at 43±2° C., 43±2° C., 42±1° C., 42±0.5° C., 43±0.5° C., 44±0.5° C., 41-45° C., or 42-44° C.

In some embodiments, the second (or later) phase amplification comprises contacting the first amplification product with a second phase amplification reaction mixture (e.g., PRO mixture) which, in combination with the first phase amplification reaction mixture, supports exponential amplification of the target nucleic acid sequence. Thus, the second phase amplification reaction mixture typically includes, at a minimum, the one or more component(s) required for exponential amplification lacking in the first phase amplification reaction mixture. In some embodiments, the second phase amplification reaction mixture comprises one or more components selected from: an amplification oligonucleotide (such as a promoter primer), a reverse transcriptase, a polymerase, a nuclease, a phosphorylase, an enzyme co-factor, a chelator, ribonucleotide triphosphates (NTPs), deoxyribonucleotide triphosphates (dNTPs), Mg²⁺, an optimal pH, an optimal temperature, a salt and a combination thereof. The polymerase can be, but is not limited to, an RNA-dependent DNA polymerase (e.g., reverse transcriptase), a DNA-dependent DNA polymerase, a DNA-dependent RNA polymerase, and a combination thereof. In some embodiments, the second phase amplification reaction mixture comprises an RNase, such as an RNase H or a reverse transcriptase with an RNase H activity. In some embodiments, the second phase amplification reaction mixture includes a promoter primer, a reverse transcriptase with an RNase H activity, and/or an RNA polymerase. In some embodiments, the second phase amplification reaction mixture further comprises a detection oligo. The detection oligo can be, but is not limited to, a Torch or molecular beacon.

In some embodiments, the Target Capture Reagent contains one or more target capture oligonucleotide and one or more T7 promoter primers, the AMP reagent contains buffer, dNTP, NTP, salt and one or more nonT7 primers, the promoter (PRO) reagent contains buffer, dNTP, NTP, salt, surfactant, one or more T7 promoter primers and one or more torch oligonucleotides, and the Enzyme (ENZ) reagent contains buffer, detergent, chelators, reverse transcriptase and DNA polymerase.

The present methods can be used to detect and/or quantify a T. vaginalis target nucleic acid sequence in a biological sample. The second phase amplification reaction can be a quantitative amplification reaction. Also described are methods for detecting the second amplification product. Detecting and/or quantifying the second amplification products may be done using a variety of detection techniques known in the art. Detection and/or quantifying can be accomplished by using, for instance, a detection probe, a sequencing reaction, electrophoresis, mass spectroscopy, melt curve analysis, or a combination thereof. In some embodiments, the second amplification product is detected and/or quantified using a detection probe. The detection probe can be, but is not limited to, a molecular torch (Torch, as described in U.S. Pat. No. 6,534,274), a molecular beacon, a hybridization switch probe, or a combination thereof. In some embodiments, the detection and/or quantification may be performed in real time. The detection probe may be included in the first and/or second phase amplification reactions with substantially equal degrees of success. The detection probe may be supplied in the first and/or second phase amplification reaction mixture (e.g., AMP mixture and/or PRO mixture). In some embodiments, the PRO mixture contains a detection probe. The detection probe can comprise a Torch.

In some embodiments, the described methods further include a step of contacting the second amplification product with another bolus of one or more amplification components selected from, but not limited to, an amplification oligonucleotide (promoter primer or non-promoter primer), a reverse transcriptase (e.g., a reverse transcriptase with an RNase H activity), a polymerase (e.g., an RNA polymerase), a nuclease, a phosphorylase, an enzyme co-factor, a chelator, ribonucleotide triphosphates (NTPs), deoxyribonucleotide triphosphates (dNTPs), Mg²⁺, a salt and a combination thereof. This additional step can provide a boost to the second phase amplification reaction as some of the amplification reaction components may become depleted.

The described methods can be used to amplify and/or detect a plurality of different target nucleic acid sequences in a sample in a multiplex reaction. In some embodiments, for a multiplex reaction, the plurality of target nucleic acid sequences are subjected to a first phase amplification reaction under conditions that do not support exponential amplification of any of the target nucleic acid sequences. The first phase amplification reaction generates a plurality of first amplification products, which are subsequently subjected to a second (and optionally later) phase amplification reaction(s) under conditions allowing exponential amplification of the first amplification products, thereby generating a plurality of second amplification products.

In some embodiments, methods are provided for amplifying a plurality of different target nucleic acid sequences in a sample, where some, but not all, of the target nucleic acid sequences are subjected to linear amplification, and/or some, but not all, of the target nucleic acid sequences are subjected to exponential amplification. At least four variants of the first phase amplification are contemplated: (1) some of the target sequences are subjected to linear amplification, and the rest are left unamplified; (2) some of the target sequences are subjected to exponential amplification, and the rest are left unamplified; (3) some of the target sequences are subjected to linear amplification, some are subjected to exponential amplification and the rest are left unamplified; and (4) some of the target sequences are subjected to linear amplification, and the rest are subjected to exponential amplification. In some embodiments, the first phase amplification may result in amplification of all of the target nucleic acid sequences (option 4) or only a subset thereof (options 1-3). The subset of the target nucleic acid sequences may represent targets known to be present in relatively low quantities and/or targets that are difficult to amplify compared to other targets. The first phase amplification reaction generates one or more first amplification product(s). The first amplification product(s) and any unamplified target nucleic acid sequence(s) in the sample are then subjected to a second phase amplification reaction under conditions allowing exponential amplification thereof, generating a plurality of second amplification products. In some embodiments, there can be more than two phases where conditions 1-4 above may apply for all phases except the final phase and where for the last phase any unamplified or linearly amplified target nucleic acid sequence(s) in the sample are subjected to an amplification reaction under conditions allowing exponential amplification thereof.

It is understood that the various optional elements and parameters discussed above in connection with multiphase uniplex (i.e. single target) amplification are also applicable to the multiphase multiplex amplification modes described herein.

C. Compositions for Multiphase Amplification of T. vaginalis

In some embodiments, a TCR mixture for capturing a T. vaginalis target nucleic acid sequence in a sample is described comprising: (a) target capture oligonucleotide (TCO) having a region that hybridizes to a target nucleic acid sequence. In some embodiments, the TCR mixture further comprises a promoter primer that hybridizes to the target nucleic acid sequence. In some embodiments, the TCR mixture optionally contains an amplification enzyme. The TCR mixture can be used to isolate and/or purify a target nucleic acid sequence from a sample. In some embodiments, the target nucleic acid is isolated as a pre-amplification hybrid containing the target nucleic acid, TCO and promoter primer.

A “target capture oligonucleotide” (TCO) comprises a nucleic acid oligonucleotide that bridges or joins a target nucleic acid and an immobilized capture probe by using binding pair members, such as, e.g., complementary nucleic acid sequences or biotin and streptavidin. In some embodiments, the target capture oligonucleotide binds nonspecifically to the target nucleic acid and immobilizes it to a solid support. The TCO contains a region of sequence complementarity, i.e., a target specific (TS) sequence, to the target nucleic acid sequence. In some embodiments, of the target capture oligonucleotide binds (hybridizes) specifically to a TCO binding sequence in the target nucleic acid. The TCO target specific sequence comprises a 10-35 nucleotide sequence having at least 90%, at least 95%, or 100% complementarity to a nucleotide sequence present in the target nucleic acid and hybridizes to a region in the target nucleic acid sequence (a TCO binding site). In some embodiments, the TCO target specific sequence is 20-30 nucleotides in length. In some embodiments, the TCO target specific sequence is 22-26 nucleotides in length and has at least 90% complementarity to a nucleotide sequence present in the target nucleic acid. The TCO target specific and TCO binding site may be perfectly complementary or there may be one or more mismatches. In both approaches the target capture oligonucleotide includes an immobilized capture probe-binding region that binds to an immobilized capture probe (e.g., by specific binding pair interaction). Members of a specific binding pair (or binding partners) are moieties that specifically recognize and bind to each other. Members may be referred to as a first binding pair member (BPM1) and second binding pair member (BPM2), which represent a variety of moieties that specifically bind together. Specific binding pairs are exemplified by, e.g., a receptor and its ligand, enzyme and its substrate, cofactor or coenzyme, an antibody or Fab fragment and its antigen or ligand, a sugar and lectin, biotin and streptavidin or avidin, a ligand and chelating agent, a protein or amino acid and its specific binding metal such as histidine and nickel, substantially complementary polynucleotide sequences, which include completely or partially complementary sequences, and complementary homopolymeric sequences. Specific binding pairs may be naturally occurring (e.g., enzyme and substrate), synthetic (e.g., synthetic receptor and synthetic ligand), or a combination of a naturally occurring BPM and a synthetic BPM. In some embodiments, the target specific sequence and the immobilized capture probe-binding region are both nucleic acid sequences. The target specific sequence and the capture probe-binding region may be covalently joined to each other, or may be on different oligonucleotides joined by one or more linkers. In some embodiments, the capture probe-binding region comprises: a poly A sequence, a poly T sequence, or a polyT-polyA sequence. In some embodiments a polyT-polyA sequence comprises dT3dA30. One or more target capture oligonucleotides may be used in target capture and/or amplication reaction. The one or more target capture oligonucleotides may bind to the same or difference target sequences. The target sequence may be from the same or difference genes and/or from the same or difference organisms.

An “immobilized capture probe” provides a means for joining a target capture oligonucleotide to a solid support. In some embodiments, an immobilized capture probe contains a base sequence recognition molecule joined to the solid support, which facilitates separation of bound target polynucleotide from unbound material. Any known solid support may be used, such as matrices and particles free in solution. For example, solid supports may be nitrocellulose, nylon, glass, polyacrylate, mixed polymers, polystyrene, silane polypropylene and magnetically attractable particles. In some embodiments, the supports include magnetic spheres that are monodisperse (i.e., uniform in size±about 5%). The immobilized capture probe may be joined directly (e.g., via a covalent linkage or ionic interaction), or indirectly to the solid support. Common examples of useful solid supports include magnetic particles or beads.

The term “target capture” refers to selectively separating or isolating a target nucleic acid from other components of a sample mixture, such as cellular fragments, organelles, proteins, lipids, carbohydrates, or other nucleic acids. A target capture system may be specific and selectively separate a predetermined target nucleic acid from other sample components (e.g., by using a sequence specific to the intended target nucleic acid, such as a TCO target specific sequence), or it may be nonspecific and selectively separate a target nucleic acid from other sample components by using other characteristics of the target (e.g., a physical trait of the target nucleic acid that distinguishes it from other sample components which do not exhibit that physical characteristic). Target capture methods and compositions have been previously described in detail (U.S. Pat. Nos. 6,110,678 and 6,534,273; and US Pub. No. 2008/0286775 A1). In some embodiments, target capture utilizes a target capture oligonucleotide in solution phase and an immobilized capture probe attached to a support to form a complex with the target nucleic acid and separate the captured target from other components.

The term “separating,” “isolating,” or “purifying” generally refers to removal of one or more components of a mixture, such as a sample, from one or more other components in the mixture. Sample components include nucleic acids in a generally aqueous solution phase, which may include cellular fragments, proteins, carbohydrates, lipids, and other compounds. In some embodiments, at least 70%, at least 75%, at least 80%, at least 85%, at least 90%, or at least 95%, of the target nucleic acid is separated or removed from other components in the mixture.

In some embodiments, the TCO comprises the nucleotide sequence of SEQ ID NO: 39, 40, or 41 or a nucleic acid sequence having at least 90% identity to SEQ ID NO: 39, 40, or 41. In some embodiments, the target specific sequence of the TCO comprises SEQ ID NO: 39, 40, or 41 or a nucleic acid sequence having at least 90% identity to SEQ ID NO: 39, 40, or 41. In some embodiments, the TCO comprises SEQ ID NO: 39, 40, or 41 or a nucleic acid sequence having at least 90% identity to SEQ ID NO: 39, 40, or 41. In some embodiments, the TCO comprises SEQ ID NO: 39. In some embodiments, the TCO comprises the nucleotide sequence of SEQ ID NO: 1, 2, or 3 or a nucleic acid sequence having at least 90% identity to SEQ ID NO: 1, 2, or 3. In some embodiments, the TCO comprises SEQ ID NO: 1, 2, or 3 or a nucleic acid sequence having at least 90% identity to SEQ ID NO: 1, 2, or 3. In some embodiments the nucleotide sequence of the TCO consists of the nucleotide sequence of SEQ ID NO: 1, 2, or 3 or a nucleic acid sequence having at least 90% identity to SEQ ID NO: 1, 2, or 3. In some embodiments, the TCO consists of SEQ ID NO: 1, 2, or 3 or a nucleic acid sequence having at least 90% identity to SEQ ID NO: 1, 2, or 3.

An “amplification oligonucleotide” (or more simply, “primer”) is an oligonucleotide that hybridizes to a target nucleic acid, or its complement, and participates in a nucleic acid amplification reaction. An amplification oligonucleotide contains at least a 3′-end that is complementary to a nucleic acid template (target nucleic acid sequence) and complexes (by hydrogen bonding or hybridization) with the template to give a primer:template complex suitable for initiation of synthesis by an RNA- or DNA-dependent polymerase. An amplification oligonucleotide is extended by the addition of covalently bonded nucleotide bases to its 3′-terminus, which bases are complementary to the template. The result is a primer extension product. Amplification oligonucleotides are at least 10 nucleotides in length. In some embodiments, the amplification oligonucleotides are least 15 nucleotides in length. In some embodiments, the amplification oligonucleotides are 15, 16, 17, 18, 19, 20, 21, 22, 23, 24, 25, 26, 27, 28, 29, or 30 or more nucleotides in length. An amplification oligonucleotide contains, at its 3′ end, a target specific (TS) sequence that is at least 90%, at least 95%, or 100% complementary to and hybridizes with a region of the target nucleic acid (amplification primer binding site). The amplification oligonucleotide target specific sequence may be perfectly complementary to a region of the target nucleic acid or it may have one or more mismatches provided the amplification oligonucleotide is capable of initiating template-dependent of synthesis by an RNA- or DNA-dependent polymerase. In some embodiments, the amplification oligonucleotide target specific sequence is at least 10 contiguous nucleotides in length. In some embodiments, the amplification oligonucleotide target specific sequence is least 15 contiguous nucleotides in length. In some embodiments, the amplification oligonucleotide target specific sequence is 15, 16, 17, 18, 19, 20, 21, 22, 23, 24, 25, 26, 27, 28, 29, or 30 contiguous nucleotides in length. The contiguous bases may be at least 90%, at least 95%, or completely (100%) complementary to the target sequence to which the amplification oligonucleotide binds. Virtually all DNA polymerases (including reverse transcriptases) that are known require complexing of an oligonucleotide to a single-stranded template (“priming”) to initiate DNA synthesis, whereas RNA replication and transcription (copying of RNA from DNA) generally do not require a primer.

In some embodiments, an amplification oligonucleotide comprises an RNA polymerase promoter sequence located 5′ of the target specific sequence. The RNA polymerase promoter sequence can be, but is not limited to, a T7, T3, or SP6 promoter sequence. Amplification oligonucleotides containing a T7 RNA polymerase promoter sequence are referred to herein as promoter primers. In some embodiments, the RNA polymerase promoter sequence is a T7 promoter sequence (T7 primers). A T7 promoter sequence can be about 25 to 30 nucleotides in length. Exemplary T7 promoter sequences include, but are not limited to, SEQ ID NO: 65 (5′-AATTTAATACGACTCACTATAGGGAGA-3′) and SEQ ID NO: 66 (5′-GAAATTAATACGACTCACTATAGGGAGA-3′).

In some embodiments, the promoter primer is a T7 primer. In some embodiments, the T7 primer comprises a nucleic acid sequence having at least 90% complementarity to a region of SEQ ID NO: 176 or a complement thereof. In some embodiments, a promoter primer contains 15-30 contiguous bases having at least 90% complementarity to a region in SEQ ID NO: 176 or a complement thereof. In some embodiments, the T7 promoter primer comprises the nucleotide sequence of SEQ ID NO: 42, 43, 44, 45, 46, 47, or 48 or a nucleic acid sequence having at least 90% identity to SEQ ID NO: 42, 43, 44, 45, 46, 47, or 48. In some embodiments, the target specific sequence of the T7 primer comprises SEQ ID NO: 42, 43, 44, 45, 46, 47, or 48 or a nucleic acid sequence having at least 90% identity to SEQ ID NO: 42, 43, 44, 45, 46, 47, or 48. In some embodiments, the T7 promoter primer comprises the nucleotide sequence of SEQ ID NO: 4, 5, 6, 7, 8, 9, 10, 11, or 12 or a nucleic acid sequence having at least 90% identity to SEQ ID NO: 4, 5, 6, 7, 8, 9, 10, 11, or 12. In some embodiments, the T7 promoter primer comprises SEQ ID NO: 4, 5, 6, 7, 8, 9, 10, 11, or 12 or a nucleic acid sequence having at least 90% identity to SEQ ID NO: 4, 5, 6, 7, 8, 9, 10, 11, or 12. In some embodiments, the nucleotide sequence of the T7 primer consists of the nucleotide sequence of SEQ ID NO: 4, 5, 6, 7, 8, 9, 10, 11, or 12 or a nucleic acid sequence having at least 90% identity to SEQ ID NO: 4, 5, 6, 7, 8, 9, 10, 11, or 12. In some embodiments, the T7 primer consists of SEQ ID NO: 4, 5, 6, 7, 8, 9, 10, 11, or 12 or a nucleic acid sequence having at least 90% identity to SEQ ID NO: 4, 5, 6, 7, 8, 9, 10, 11, or 12.

A promoter primer (e.g., T7 primer) binds specifically to the target nucleic acid at its target sequence and a reverse transcriptase (RT) extends the 3′ end of the promoter primer using the target strand as a template to create a cDNA copy, resulting in a RNA:cDNA duplex. RNase activity (e.g., RNase H of RT enzyme) digests the RNA of the RNA:cDNA duplex.

In some embodiments, a first phase amplification mixture (AMP mixture) for linear amplification of a T. vaginalis target nucleic acid sequence comprises: a non-RNA polymerase promoter-containing oligonucleotide (also termed non-promoter primer or NT7 primer); a reverse transcriptase, an RNA polymerase, dNTPs, and NTPs, wherein the first phase amplification mixture is lacking in at least one component necessary for exponential amplification. The RNA polymers can be a T7 RNA polymerase. The AMP mixture additionally contains necessary components necessary to amplify the target nucleic acid during a linear first phase amplification reaction provided the at least one component required for exponential amplification of the target nucleic acid sequence is no present. In some embodiments, the lacking at least one component necessary for exponential amplification is additional promoter primer.

In some embodiments, the NT7 primer comprises a nucleic acid sequence having at least 90% complementarity to a region of SEQ ID NO: 177 or a complement thereof. In some embodiments, an NT7 primer contains 15-30 contiguous bases having at least 90% complementarity to a region in SEQ ID NO: 177 or a complement thereof. In some embodiments, the non-promoter primer comprises the nucleotide sequence of SEQ ID NO: 49, 50, 51, 52, 53, 54, or 55 or a nucleic acid sequence having at least 90% identity to SEQ ID NO: 49, 50, 51, 52, 53, 54, or 55. In some embodiments, the non-promoter primer comprises the nucleotide sequence of SEQ ID NO: 13, 14, 15, 16, 17, 18, or 19 or a nucleic acid sequence having at least 90% identity to SEQ ID NO: 13, 14, 15, 16, 17, 18, or 19. In some embodiments, the non-promoter primer comprises SEQ ID NO: 13, 14, 15, 16, 17, 18, or 19 or a nucleic acid sequence having at least 90% identity to SEQ ID NO: 13, 14, 15, 16, 17, 18, or 19. In some embodiments, the nucleotide sequence of the non-promoter primer consists of the nucleotide sequence of SEQ ID NO: 13, 14, 15, 16, 17, 18, or 19 or a nucleic acid sequence having at least 90% identity to SEQ ID NO: 13, 14, 15, 16, 17, 18, or 19. In some embodiments the non-promoter primer consists of SEQ ID NO: 13, 14, 15, 16, 17, 18, or 19 or a nucleic acid sequence having at least 90% identity to SEQ ID NO: 13, 14, 15, 16, 17, 18, or 19.

A “detection oligonucleotide,” “detection probe,” or “probe” is an oligonucleotide that hybridizes specifically to a target sequence, such as an amplification product, under conditions that promote nucleic acid hybridization, for detection of the target nucleic acid or its amplification product. Detection may either be direct (i.e., detection oligonucleotide hybridized directly to the target) or indirect (i.e., a detection oligonucleotide hybridized to an intermediate structure that links the detection oligonucleotide to the target). A detection oligonucleotide's target sequence generally refers to a specific sequence within a larger sequence which the detection oligonucleotide hybridizes specifically. A detection oligonucleotide may include target specific sequences and a non-target-complementary sequence. Such non-target-complementary sequences can include sequences which will confer a desired secondary or tertiary structure, such as a hairpin structure, which can be used to facilitate detection and/or amplification. (e.g., U.S. Pat. Nos. 5,118,801; 5,312,728; 5,925,517; 6,150,097; 6,849,412; 6,835,542; 6,534,274; and 6,361,945; and US Patent Application Pub. Nos. 20060068417A1 and 20060194240A1). The complementary and non-complementary sequences can be contiguous or joined by a linker. In some embodiments, the linker is a C₁, C₂, C₃, C₄, C₅, C₆, C₇, C₈, C₉, C₁₀, C₁₁, C₁₂, C₁₃, C₁₄, C₁₅, or C₁₆ linker. In some embodiments, the linker is a C9 linker. A detection oligonucleotide can be RNA, DNA, contain one or more modified nucleotides, or a combination thereof. In some embodiments, a detection oligonucleotide contains one ore more 2′ methoxy nucleotides. In some embodiments, a detection oligonucleotide contains all 2′ methoxy ribonucleotides.

In some embodiments, a detection oligonucleotide contains a one or more detectable markers or labels. A detectable marker can be, but is not limited, to a fluorescent molecule. The fluorescent molecule can be attached to the 5′ or 3′ end of the detection oligonucleotide or anywhere along the oligomer. In some embodiments a detection oligonucleotide can be a molecular beacon or torch. In some embodiments, a detection oligonucleotide can be a hydrolysis detection oligonucleotide. A detection oligonucleotide can contain a fluorescent molecule attached to the 5′ end and a quencher attached to the 3′ end. Alternatively, a fluorescent molecule can be attached to the 3′ end of the detection oligonucleotide and a quencher attached to the 5′ end of the detection oligonucleotide.

“Label” or “detectable label” refers to a moiety or compound joined directly or indirectly to a detection oligonucleotide that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s), which amplify a detectable signal. Any detectable moiety may be used, e.g., radionuclide, ligand such as biotin or avidin, enzyme, enzyme substrate, reactive group, chromophore such as a dye or particle (e.g., latex or metal bead) that imparts a detectable color, luminescent compound (e.g. bioluminescent, phosphorescent, or chemiluminescent compound), and fluorescent compound (i.e., fluorophore). Fluorophores include, but are not limited to, FAM™, TET™, CAL FLUOR™ (Orange or Red), QUASAR™, fluorescein, hexochloro-Fluorescein (HEX), rhodamine, Carboxy-X-Rhodamine (ROX), tetramethylrhodamine, IAEDANS, EDANS, DABCYL, coumarin, BODIPY FL, lucifer yellow, eosine, erythrosine, Texas Red, ROX, CY dyes (such as CY5), Cyanine 5.5 (Cy5.5) and fluorescein/QSY7 dye compounds. In some embodiments, detection oligonucleotide comprises a base spacer between the 5′ end of the oligonucleotide and the label. The spacer (or linker) can be an alkyl group. Fluorophores may be used in combination with a quencher molecule that absorbs light when in close proximity to the fluorophore to diminish background fluorescence. Such quenchers include, but are not limited to, BLACKBERRY® quencher (BBQ-650®), BLACK HOLE QUENCHER™ (or BHQ™, including, but not limited to, Black Hole Quencher-2 (BHQ2)) or TAMRA™ compounds. Examples of interacting donor/acceptor label pairs that may be used in connection with the disclosure, making no attempt to distinguish FRET from non-FRET pairs, include, but are not limited to, fluorescein/tetramethylrhodamine, IAEDANS/fluororescein, EDANS/DABCYL, coumarin/DABCYL, fluorescein/fluorescein, BODIPY FL/BODIPY FL, fluorescein/DABCYL, CalRed-610/BHQ-2, lucifer yellow/DABCYL, Quasar 750/BHQ-2, BODIPY/DABCYL, eosine/DABCYL, erythrosine/DABCYL, tetramethylrhodamine/DABCYL, Texas Red/DABCYL, CY5/BHQ1, CY5/BHQ2, CY3/BHQ1, CY3/BHQ2 and fluorescein/QSY7 dye. In some embodiments, a detection oligonucleotide contains a label that is detectable in a homogeneous system in which bound labeled detection oligonucleotide in a mixture exhibits a detectable change compared to unbound labeled detection oligonucleotide, which allows the label to be detected without physically removing hybridized from unhybridized labeled detection oligonucleotide (e.g., U.S. Pat. Nos. 5,283,174, 5,656,207, and 5,658,737). Detectable labels or detection oligonucleotides known in the art include, but are not limited to, chemiluminescent labels, (including acridinium ester compounds, U.S. Pat. Nos. 5,656,207, 5,658,737, and 5,639,604) TaqMan™ probes, molecular torches, and molecular beacons. TaqMan™ probes include a donor and acceptor label wherein fluorescence is detected upon enzymatically degrading the detection oligonucleotide during amplification in order to release the fluorophore from the presence of the quencher. Molecular torches and beacons exist in open and closed configurations wherein the closed configuration quenches the fluorophore and the open position separates the fluorophore from the quencher to allow fluorescence. Hybridization to target opens the otherwise closed detection oligonucleotides.

In some embodiments, the detection probe is a Torch. In some embodiments, the Torch comprises a nucleic acid sequence having at least 90% complementarity to a region of SEQ ID NO: 178 or a complement thereof. In some embodiments, a promoter primer contains 10-30 contiguous bases having at least 90% complementarity to a region in SEQ ID NO: 177 or a complement thereof. In some embodiments, the Torch comprises the nucleotide sequence of SEQ ID NO: 56, 57, 58, 59, 60, 61, or 62 or a nucleic acid sequence having at least 90% identity to SEQ ID NO: 56, 57, 58, 59, 60, 61, or 62. In some embodiments, the Torch comprises the nucleotide sequence of SEQ ID NO: 20, 21, 22, 23, 24, 25, 26, 27, or 28 or a nucleic acid sequence having at least 90% identity to SEQ ID NO: 20, 21, 22, 23, 24, 25, 26, 27, or 28. In some embodiments, Torch comprises SEQ ID NO: 20, 21, 22, 23, 24, 25, 26, 27, or 28 or a nucleic acid sequence having at least 90% identity to SEQ ID NO: 20, 21, 22, 23, 24, 25, 26, 27, or 28. In some embodiments, the nucleotide sequence of the Torch consists of the nucleotide sequence of SEQ ID NO: 20, 21, 22, 23, 24, 25, 26, 27, or 28 or a nucleic acid sequence having at least 90% identity to SEQ ID NO: 20, 21, 22, 23, 24, 25, 26, 27, or 28. In some embodiments the Torch consists of SEQ ID NO: 20, 21, 22, 23, 24, 25, 26, 27, or 28 or a nucleic acid sequence having at least 90% identity to SEQ ID NO: 20, 21, 22, 23, 24, 25, 26, 27, or 28. In some embodiments, the torch contains a fluorescent molecule attached to the 5′ end and a quencher attached to the 3′ end. Alternatively, a fluorescent molecule can be attached to the 3′ end of the torch and a quencher attached to the 5′ end of the detection oligonucleotide. In some embodiments, the torch contains a 5-6 nucleotide sequence at the 3′ end that is complementary to and can hybridize with 5-6 nucleotide at the 5′ end. In some embodiments, the 5-6 nucleotide sequence at the 3′ end that is complementary to and can hybridize with 5-6 nucleotide at the 5′ end are linked to the torch via a linker. In some embodiments, the linker is a C₁₋₁₆ linker. In some embodiments, the linker is a C₉ linker.

“Detection” of the amplified products may be accomplished using any known method. For example, the amplified nucleic acids may be associated with a surface that results in a detectable physical change (e.g., an electrical change). Amplified nucleic acids may be detected in solution phase or by concentrating them in or on a matrix and detecting labels associated with them (e.g., an intercalating agent such as ethidium bromide or cyber green). Other detection methods use probes complementary to a sequence in the amplified product and detect the presence of the probe:product complex, or use a complex of probes to amplify the signal detected from amplified products (e.g., U.S. Pat. Nos. 5,424,413, 5,451,503 and 5,849,481). Other detection methods use a probe in which signal production is linked to the presence of the target sequence because a change in signal results only when the labeled probe binds to amplified product, such as in a molecular beacon, molecular torch, or hybridization switch probe (e.g., U.S. Pat. Nos. 5,118,801, 5,312,728, 5,925,517, 6,150,097, 6,361,945, 6,534,274, 6,835,542, 6,849,412 and 8,034,554; and U.S. Pub. No. 2006/0194240 A1). Detection can be achieved using detection oligonucleotides that are present during target amplification and hybridize to the amplicon in real time. A detection oligonucleotide may contain a fluorophore and a quencher. Torches contain complementary regions at each end. These complementary regions bind to each other and form a “closed” torch. In the closed configuration, the fluorophore and quencher are in close proximity and the fluorophore signal is quenched. That is, it does not emit a detectable signal when excited by light. However, when the torch binds to the complementary target, the complementary regions within the torch are forced apart to form an “open” torch. In the open form, the fluorophore and quencher are not in close proximity and the fluorophore signal is detectable when excited (i.e., no longer quenched). Amplicon-torch binding results in the separation of the quencher from the fluorophore; which allows fluorophore excitation in response to light stimulus and signal emission at a specific wavelength. The torches can be present during amplification and bind to the complementary amplicon as it is generated in real time. As more amplicon is created, more torch is bound and more signal is created. The signal eventually reaches a level that it can be detected above the background and ultimately reaches a point where all available torch is bound to amplicon and the signal reaches a maximum. At the start of amplification, and low copy number of the amplified sequence, most of the detection oligonucleotide is closed (the 3′ and 5′ ends are base paired, and the fluorescent signal is quenched. During amplification, more detection oligonucleotide binds to target sequence, thus separating the 3′ and 5′ ends of the detection oligonucleotide, leading to increases fluorescence (decreased quenching of fluorescence). After further amplification, the fluorescent signal approaches a maximum.

In some embodiments, detection is performed at time intervals. Detection can be done by measuring fluorescence at regular time intervals. Time intervals can be, but are not limited to: 1-60 sec, 1-120 sec, 1-180 sec, 1-240 sec, or 1-300 sec. In some embodiments, the time interval is 5, 10, 15, 20, 25, 30, 35, 40, 45, 50, 55, or 60 sec. For detection performed at regular time intervals, each interval is referred to as a cycle. Detection can be performed for 20-240 cycles, 30-210 cycles, 40-180 cycles, 50-150 cycles, or 60-120 cycles. For example, detection every 30 sec for 60 minutes constitutes 120 cycles. Detection may occur at the beginning or end of a cycle. Detection can also be performed continuously.

In some embodiments, an amplification oligonucleotide (promoter primer or non-promoter primer), detection oligonucleotide, or target capture oligonucleotide contains one or more modified nucleotides. An oligonucleotide can have 1, 2, 3, 4, 5, 6, 7, 8, or more modified nucleotides. In some embodiments, more than 50%, more than 60%, more than 70%, more than 75%, more than 80%, more than 85%, more than 90%, more than 950%, or 100% of the nucleotides are modified. Modified nucleotides include nucleotides having modified nucleobases. Modified nucleobases include, but are not limited to, synthetic and natural nucleobases, 5-substituted pyrimidines, 6-azapyrimidines, and N-2, N-6 and O-6 substituted purines. Modified nucleotides also include nucleotides with a modified base, including, but not limited to, 2′-modified nucleotides (including, but not limited to 2′-O-methyl nucleotides and 2′-halogen nucleotides, such as 2′-fluoro nucleotides). Modified nucleotides also include nucleotides with modified linkages, such as, but not limited phosphorothioate linkages.

Any of the oligonucleotides described herein can contain one or more tags. A “tag” can be a nucleotide sequence covalently attached to an oligonucleotide for the purpose of conferring some additional functionality beyond binding to the target sequence. Non-limiting examples of oligonucleotide tags include a 5′ promoter for an RNA polymerase, a primer binding site, a sequencing tag, a mass tag, a bar code tag, a capture tag, and so forth (e.g., U.S. Pat. Nos. 5,422,252, 5,882,856, 6,828,098, and PCT Pub. No. 05/019479). A tag can also be a non-nucleotide molecule covalently attached to an oligonucleotide for the purpose of conferring some additional functionality.

Where multiplex amplification is intended, the present composition may include a plurality of different target capture oligonucleotides promoter primers, and non-promoter primers that hybridize to a plurality of different target nucleic acid sequences. The different target nucleic acid sequences may be in the same or different organisms.

As noted above, methods and compositions disclosed herein are useful for amplifying target nucleic acid sequences in vitro to produce amplified sequences that can be detected to indicate the presence of the target nucleic acid in a sample. The methods and compositions are useful for synthesizing amplified nucleic acids to provide useful information for making diagnoses and/or prognoses of medical conditions, detecting the purity or quality of environmental and/or food samples, or investigating forensic evidence. The methods and compositions are advantageous in providing highly sensitive assays over a wide dynamic range that are relatively rapid and inexpensive to perform, making them suitable for use in high throughput and/or automated systems. The methods and compositions can be used for assays that analyze single target sequences, i.e., uniplex amplification systems, and are especially useful for assays that simultaneously analyze multiple different target sequences, i.e., multiplex amplification systems. In some embodiments, compositions and reactions mixtures are provided in kits that include defined assay components that are useful because they allow a user to efficiently perform methods that use the components together in an assay to amplify desired targets.

D. Oligonucleotide Compositions for Multiphase Amplification and Detection of T. vaginalis.

In some embodiments, the TCO comprises the nucleotide sequence of SEQ ID NO: 41, the T7 primer comprises the nucleotide sequence of SEQ ID NO: 47, the NT7 primer comprises the nucleotide sequence of SEQ ID NO: 51, and the Torch comprises the nucleotide sequence of SEQ ID NO: 58.

In some embodiments, the TCO comprises the nucleotide sequence of SEQ ID NO: 3, the T7 primer comprises the nucleotide sequence of SEQ ID NO: 11, the NT7 primer comprises the nucleotide sequence of SEQ ID NO: 15, and the Torch comprises the nucleotide sequence of SEQ ID NO: 24.

In some embodiments, the TCO comprises the nucleotide sequence of SEQ ID NO: 41, the T7 primer comprises the nucleotide sequence of SEQ ID NO: 42, the NT7 primer comprises the nucleotide sequence of SEQ ID NO: 50, and the Torch comprises the nucleotide sequence of SEQ ID NO: 56.

In some embodiments, the TCO comprises the nucleotide sequence of SEQ ID NO: 3, the T7 primer comprises the nucleotide sequence of SEQ ID NO: 4, the NT7 primer comprises the nucleotide sequence of SEQ ID NO: 14, and the Torch comprises the nucleotide sequence of SEQ ID NO: 20.

In some embodiments, the TCO comprises the nucleotide sequence of SEQ ID NO: 41, the T7 primer comprises the nucleotide sequence of SEQ ID NO: 42, the NT7 primer comprises the nucleotide sequence of SEQ ID NO: 50, and the Torch comprises the nucleotide sequence of SEQ ID NO: 57.

In some embodiments, the TCO comprises the nucleotide sequence of SEQ ID NO: 3, the T7 primer comprises the nucleotide sequence of SEQ ID NO: 4, the NT7 primer comprises the nucleotide sequence of SEQ ID NO: 14, and the Torch comprises the nucleotide sequence of SEQ ID NO: 21.

In some embodiments, the TCO comprises the nucleotide sequence of SEQ ID NO: 41, the T7 primer comprises the nucleotide sequence of SEQ ID NO: 42, the NT7 primer comprises the nucleotide sequence of SEQ ID NO: 49, and the Torch comprises the nucleotide sequence of SEQ ID NO: 57.

In some embodiments, the TCO comprises the nucleotide sequence of SEQ ID NO: 3, the T7 primer comprises the nucleotide sequence of SEQ ID NO: 4, the NT7 primer comprises the nucleotide sequence of SEQ ID NO: 13, and the Torch comprises the nucleotide sequence of SEQ ID NO: 21.

In some embodiments, the TCO comprises the nucleotide sequence of SEQ ID NO: 41, the T7 primer comprises the nucleotide sequence of SEQ ID NO: 45, the NT7 primer comprises the nucleotide sequence of SEQ ID NO: 51, and the Torch comprises the nucleotide sequence of SEQ ID NO: 58.

In some embodiments, the TCO comprises the nucleotide sequence of SEQ ID NO: 3, the T7 primer comprises the nucleotide sequence of SEQ ID NO: 9, the NT7 primer comprises the nucleotide sequence of SEQ ID NO: 15, and the Torch comprises the nucleotide sequence of SEQ ID NO: 23.

In some embodiments, the TCO comprises the nucleotide sequence of SEQ ID NO: 40, the T7 primer comprises the nucleotide sequence of SEQ ID NO: 42, the NT7 primer comprises the nucleotide sequence of SEQ ID NO: 50, and the Torch comprises the nucleotide sequence of SEQ ID NO: 56.

In some embodiments, the TCO comprises the nucleotide sequence of SEQ ID NO: 2, the T7 primer comprises the nucleotide sequence of SEQ ID NO: 4, the NT7 primer comprises the nucleotide sequence of SEQ ID NO: 14, and the Torch comprises the nucleotide sequence of SEQ ID NO: 20.

In some embodiments, the TCO comprises the nucleotide sequence of SEQ ID NO: 40, the T7 primer comprises the nucleotide sequence of SEQ ID NO: 42, the NT7 primer comprises the nucleotide sequence of SEQ ID NO: 50, and the Torch comprises the nucleotide sequence of SEQ ID NO: 57.

In some embodiments, the TCO comprises the nucleotide sequence of SEQ ID NO: 2, the T7 primer comprises the nucleotide sequence of SEQ ID NO: 4, the NT7 primer comprises the nucleotide sequence of SEQ ID NO: 14, and the Torch comprises the nucleotide sequence of SEQ ID NO: 21.

In some embodiments, the TCO comprises the nucleotide sequence of SEQ ID NO: 40, the T7 primer comprises the nucleotide sequence of SEQ ID NO: 42, the NT7 primer comprises the nucleotide sequence of SEQ ID NO: 49, and the Torch comprises the nucleotide sequence of SEQ ID NO: 57.

In some embodiments, the TCO comprises the nucleotide sequence of SEQ ID NO: 2, the T7 primer comprises the nucleotide sequence of SEQ ID NO: 4, the NT7 primer comprises the nucleotide sequence of SEQ ID NO: 13, and the Torch comprises the nucleotide sequence of SEQ ID NO: 21.

In some embodiments, the TCO comprises the nucleotide sequence of SEQ ID NO: 40, the T7 primer comprises the nucleotide sequence of SEQ ID NO: 45, the NT7 primer comprises the nucleotide sequence of SEQ ID NO: 51, and the Torch comprises the nucleotide sequence of SEQ ID NO: 58.

In some embodiments, the TCO comprises the nucleotide sequence of SEQ ID NO: 2, the T7 primer comprises the nucleotide sequence of SEQ ID NO: 9, the NT7 primer comprises the nucleotide sequence of SEQ ID NO: 15, and the Torch comprises the nucleotide sequence of SEQ ID NO: 23.

In some embodiments, the TCO comprises the nucleotide sequence of SEQ ID NO: 40, the T7 primer comprises the nucleotide sequence of SEQ ID NO: 42, the NT7 primer comprises the nucleotide sequence of SEQ ID NO: 49, and the Torch comprises the nucleotide sequence of SEQ ID NO: 56.

In some embodiments, the TCO comprises the nucleotide sequence of SEQ ID NO: 2, the T7 primer comprises the nucleotide sequence of SEQ ID NO: 4, the NT7 primer comprises the nucleotide sequence of SEQ ID NO: 13, and the Torch comprises the nucleotide sequence of SEQ ID NO: 20.

Additional oligonucleotides are provided in Table 1.

TABLE 1
Oligonucleotide and T. vaginalis target sequences.

| SEQ ID | | |
|---|---|---|
| NO. | Sequence (5′ → 3′) | Type |
| 1 | GCCTGCTGCTACCCGTGGATATTTTAAAAAAAAAAAAAAAAAAAAAAAAAAAAAA | TCO |
| 2 | CTACCAGGGTCTCTAATCCTGTTGGATTTAAAAAAAAAAAAAAAAAAAAAAAAAAAAAAA | TCO |
| 3 | AATCAACGCTAGACAGGTCAACCCTTTAAAAAAAAAAAAAAAAAAAAAAAAAAAAAA | TCO |
| 4 | AATTTAATACGACTCACTATAGGGAGATAACCGAAGGACTTCGGCAAAGTAA | T7 |
| 5 | AATTTAATACGACTCACTATAGGGAGAGCTACCCTCTTCCACCTGC | T7 |
| 6 | AATTTAATACGACTCACTATAGGGAGAGGCATCACGGACCTGTTATTGC | T7 |
| 7 | GCAAT(L)*AATTTAATACGACTCACTATAGGGAGAGGCATCACGGACCTGTTATTGC | T7 |
| 8 | GCAATA(L)*AATTTAATACGACTCACTATAGGGAGAGGCATCACGGACCTGTTATTGC | T7 |
| 9 | AATTTAATACGACTCACTATAGGGAGAGCTCGCAGTCCTATTGATCCTAA | T7 |
| 10 | AATTTAATACGACTCACTATAGGGAGAGCACCCTCTCAGGCTCGC | T7 |
| 11 | AATTTAATACGACTCACTATAGGGAGAGTAGCGCACCCTCTCAGGCTCG | T7 |
| 12 | AATTTAATACGACTCACTATAGGGAGAGTTCATGACGCTGATTACAAACG | T7 |
| 13 | GGCTTCGGGTCTTTCAGGATATTGT | NT7 |
| 14 | CGGGTCTTTCAGGATATTGT | NT7 |
| 15 | GCTAACGAGCGAGATTATCGCC | NT7 |
| 16 | GGTAGCAATAACAGGTCCGTG | NT7 |
| 17 | GGTCCGTGATGCCCTTTAGATG | NT7 |
| 18 | CGTGATGCCCTTTAGATGCTCTG | NT7 |
| 19 | CGTGATGCCCTTTAGATGCTCTGG | NT7 |
| 20 | GCCGUUGGUGGUGC(L)*ACGGC | Torch |
| 21 | GCGUUGAUUCAGC(L)*ACGC | Torch |
| 22 | CGAAGUCCUUCGGUUAAAGUUC(L)*CUUCG | Torch |
| 23 | CGAAGUCCUUCGGUUAAAGUUC(L)*ACUUCG | Torch |
| 24 | CGAAGUCCUUCGGUUAAAGUUC(L)*CUUCG | Torch |
| 25 | UUCGGUUAAAGUUCUAAUUGGGACU(L)*CCGAA | Torch |
| 26 | UUCGGUUAAAGUUCUAAUUGGGAC(L)*ACCGAA | Torch |
| 27 | GCGUGCUACAAUGUUAGGAUCA(L)*CACGC | Torch |
| 28 | GACUGCGAGCCUGAGAGGGUG(L)*ACGUC | Torch |
| 29 | GATGGAGCGTACCACCGTTTAAAAAAAAAAAAAAAAAAAAAAAAAAAAAA | Candida TCO |
| 30 | AGATCGGTATCGGGTGCTTGTTTAAAAAAAAAAAAAAAAAAAAAAAAAAAAAA | Candida TCO |
| 31 | GCTCAGAAAACCAGAAGCGAAACGGGTTTAAAAAAAAAAAAAAAAAAAAAAAAAAAAAA | Candida TCO |
| 32 | AATTTAATACGACTCACTATAGGGAGATCAAGTTCGCATATTGCAC | Candida T7 |
| 33 | AATTTAATACGACTCACTATAGGGAGAATACTGGGCCGACATCCTTACG | Candida T7 |
| 34 | GGTAGTTTGGCTTTTCTTTGG | Candida NT7 |
| 35 | CGTTACAAGAAATATACACGG | Candida NT7 |
| 36 | GCATTGGAGTTTCTGCTG | Candida NT7 |
| 37 | GGAUGUGACUGUCAUGC(L)*CAUCC | Candida |
| | | Torch |
| 38 | GGAAUGGCGCCGUGGAUGGUUG(L)*CAUUCC | Candida |
| | | Torch |
| 39 | GCCTGCTGCTACCCGTGGATAT | |
| 40 | CTACCAGGGTCTCTAATCCTGTTGGA | |
| 41 | AATCAACGCTAGACAGGTCAACCC | |
| 42 | TAACCGAAGGACTTCGGCAAAGTAA | |
| 43 | GCTACCCTCTTCCACCTGC | |
| 44 | GGCATCACGGACCTGTTATTGC | |
| 45 | GCTCGCAGTCCTATTGATCCTAA | |
| 46 | GCACCCTCTCAGGCTCGC | |
| 47 | GTAGCGCACCCTCTCAGGCTCG | |
| 48 | GTTCATGACGCTGATTACAAACG | |
| 49 | GGCTTCGGGTCTTTCAGGATATTGT | |
| 50 | CGGGTCTTTCAGGATATTGT | |
| 51 | GCTAACGAGCGAGATTATCGCC | |
| 52 | GGTAGCAATAACAGGTCCGTG | |
| 53 | GGTCCGTGATGCCCTTTAGATG | |
| 54 | CGTGATGCCCTTTAGATGCTCTG | |
| 55 | CGTGATGCCCTTTAGATGCTCTGG | |
| 56 | GCCGUUGGUGGUGC | |
| 57 | GCGUUGAUUCAGC | |
| 58 | CGAAGUCCUUCGGUUAAAGUUC | |
| 59 | UUCGGUUAAAGUUCUAAUUGGGACU | |
| 60 | UUCGGUUAAAGUUCUAAUUGGGAC | |
| 61 | GCGUGCUACAAUGUUAGGAUCA | |
| 62 | GACUGCGAGCCUGAGAGGGUG | |
| 63 | GCAUG(L)*GUGCGAAUUGGGACAUGC | Torch |
| 64 | GAAGGU(L)*UACUUUGCCGAAGUCCUUCG | Torch |
| 65 | AATTTAATACGACTCACTATAGGGAGA | T7 promoter |
| 66 | GAAATTAATACGACTCACTATAGGGAGA | T7 promoter |
| 67 | TTGCCGAAGTCCTTCGGTTAAAGTTCTAATTG | |
| 68 | UUGCCGAAGUCCUUCGGUUAAAGUUCUAAUUG | |
| 69 | CAATTAGAACTTTAACCGAAGGACTTCGGCAA | |
| 70 | CAAUUAGAACUUUAACCGAAGGACUUCGGCAA | |
| 71 | TGCCGAAGTCCTTCGGTTAAAGTTCTAATTGG | |
| 72 | UGCCGAAGUCCUUCGGUUAAAGUUCUAAUUGG | |
| 73 | CCAATTAGAACTTTAACCGAAGGACTTCGGCA | |
| 74 | CCAAUUAGAACUUUAACCGAAGGACUUCGGCA | |
| 75 | GCCGAAGTCCTTCGGTTAAAGTTCTAATTGGG | |
| 76 | GCCGAAGUCCUUCGGUUAAAGUUCUAAUUGGG | |
| 77 | CCCAATTAGAACTTTAACCGAAGGACTTCGGC | |
| 78 | CCCAAUUAGAACUUUAACCGAAGGACUUCGGC | |
| 79 | CCGAAGTCCTTCGGTTAAAGTTCTAATTGGG | |
| 80 | CCGAAGUCCUUCGGUUAAAGUUCUAAUUGGG | |
| 81 | CCCAATTAGAACTTTAACCGAAGGACTTCGG | |
| 82 | CCCAAUUAGAACUUUAACCGAAGGACUUCGG | |
| 83 | CGAAGTCCTTCGGTTAAAGTTCTAATTGGGAC | |
| 84 | CGAAGUCCUUCGGUUAAAGUUCUAAUUGGGAC | |
| 85 | GTCCCAATTAGAACTTTAACCGAAGGACTTCG | |
| 86 | GUCCCAAUUAGAACUUUAACCGAAGGACUUCG | |
| 87 | CGAAGTCNTTCGGTTAAAGTTCTAATTGGGAC | |
| 88 | CGAAGUCNUUCGGUUAAAGUUCUAAUUGGGAC | |
| 89 | GTCCCAATTAGAACTTTAACCGAANGACTTCG | |
| 90 | GUCCCAAUUAGAACUUUAACCGAANGACUUCG | |
| 91 | GAAGTCCTTCGGTTAAAGTTCTAA | |
| 92 | GAAGUCCUUCGGUUAAAGUUCUAA | |
| 93 | TTAGAACTTTAACCGAAGGACTTC | |
| 94 | UUAGAACUUUAACCGAAGGACUUC | |
| 95 | GTCCTTCGGTTAAAGTTCTAATTGG | |
| 96 | GUCCUUCGGUUAAAGUUCUAAUUGG | |
| 97 | CCAATTAGAACTTTAACCGAAGGAC | |
| 98 | CCAAUUAGAACUUUAACCGAAGGAC | |
| 99 | TTCGGTTAAAGTTCTAATTGGGACTCCCTGCG | |
| 100 | UUCGGUUAAAGUUCUAAUUGGGACUCCCUGCG | |
| 101 | CGCAGGGAGTCCCAATTAGAACTTTAACCGAA | |
| 102 | CGCAGGGAGUCCCAAUUAGAACUUUAACCGAA | |
| 103 | TTGCCGAAGTCCTTCGGTTAAAGTTCTAATTGGGACTCCCTGCG | |
| 104 | UUGCCGAAGUCCUUCGGUUAAAGUUCUAAUUGGGACUCCCUGCG | |
| 105 | CGCAGGGAGTCCCAATTAGAACTTTAACCGAAGGACTTCGGCAA | |
| 106 | CGCAGGGAGUCCCAAUUAGAACUUUAACCGAAGGACUUCGGCAA | |
| 107 | TTCGGTTAAAGTTCTAA | |
| 108 | UUCGGUUAAAGUUCUAA | |
| 109 | TTAGAACTTTAACCGAA | |
| 110 | UUAGAACUUUAACCGAA | |
| 111 | GCTAACGAGCGAGATTATCGCC | |
| 112 | GCUAACGAGCGAGAUUAUCGCC | |
| 113 | GGCGATAATCTCGCTCGTTAGC | |
| 114 | GGCGAUAAUCUCGCUCGUUAGC | |
| 115 | GGCATCACGGACCTGTTATTGC | |
| 116 | GGCAUCACGGACCUGUUAUUGC | |
| 117 | GCAATAACAGGTCCGTGATGCC | |
| 118 | GCAAUAACAGGUCCGUGAUGCC | |
| 119 | AATTTAATACGACTCACTATAGGGAGAGGCATCACGGACCTGTTATTGC | |
| 120 | AATTTAATACGACTCACTATAGGGAGA | |
| 121 | GCCTGCTGCTACCCGTGGATAT | |
| 122 | GCCUGCUGCUACCCGUGGAUAU | |
| 123 | ATATCCACGGGTAGCAGCAGGC | |
| 124 | AUAUCCACGGGUAGCAGCAGGC | |
| 125 | GCCTGCTGCTACCCGTGGATATTTTAAAAAAAAAAAAAAAAAAAAAAAAAAAAAA | |
| 126 | TTTAAAAAAAAAAAAAAAAAAAAAAAAAAAAAAA | |
| 127 | GCTAACGAGCGAGATTATCGCCAAGCAATAACAGGTCCGTGATG | |
| 128 | GTCCCAATTAGAACTTTAACCGAAGGACTTCGGCAA | |
| 129 | CGCAGGGAGTCCCAATTAGAACTTTAACCGAA | |
| 130 | CAATTAGAACTTTAACCGAAG | |
| 131 | TTGCTTGGCGATAATCTCGCTCG | |
| 132 | CCTGTTATTGCTTGGCGATAATCTCGC | |
| 133 | CGGACCTGTTATTGCTTGGCGATAATCTC | |
| 134 | GCCTCTCGGCTTTGCAGTCCTATT | |
| 135 | GTTGATCCTGCCAAG | |
| 136 | GCCATGCAAGTGTTAG | |
| 137 | CCATTCGACTGAGTGACCTATC | |
| 138 | GATTCCTGGTTCATGACGCTG | |
| 139 | CCGAGTCATCCAATCG | |
| 140 | CCTACCGTTACCTTGTTACGAC | |
| 141 | GAAGUCCUUCGGUUAAAGUUCUAA | |
| 142 | GUCCUUCGGUUAAAGUUCUAAUUGG | |
| 143 | GTGCGTGGGTTGACCTGTCTAGCGTTGATT | |
| 144 | GUGCGUGGGUUGACCUGUCUAGCGUUGAUU | |
| 145 | AATCAACGCTAGACAGGTCAACCCACGCAC | |
| 146 | AAUCAACGCUAGACAGGUCAACCCACGCAC | |
| 147 | GACCTGTCTA | |
| 148 | GACCUGUCUA | |
| 149 | TAGACAGGTC | |
| 150 | UAGACAGGUC | |
| 151 | CTAGACAGGTCAACCCACGCAC | |
| 152 | CUAGACAGGUCAACCCACGCAC | |
| 153 | GTGCGTGGGTTGACCTGTCTAG | |
| 154 | GUGCGUGGGUUGACCUGUCUAG | |
| 155 | CUAGACAGGUCAACCCACGCACTTTAAAAAAAAAAAAAAAAAAAAAAAAAAAAAA | |
| 156 | AATCAACGCTAGACAGGTCAACCC | |
| 157 | AAUCAACGCUAGACAGGUCAACCC | |
| 158 | GGGTTGACCTGTCTAGCGTTGATT | |
| 159 | GGGUUGACCUGUCUAGCGUUGAUU | |
| 160 | AATCAACGCTAGACAGGTCAACCCTTTAAAAAAAAAAAAAAAAAAAAAAAAAAAAAA | |
| 161 | TCAACGCTAGACAGGTCAA | |
| 162 | UCAACGCUAGACAGGUCAA | |
| 163 | TTGACCTGTCTAGCGTTGA | |
| 164 | UUGACCUGUCUAGCGUUGA | |
| 165 | TCAACGCTAGACAGGTCAATTTAAAAAAAAAAAAAAAAAAAAAAAAAAAAAA | VVVV |
| 166 | AATCAACGCTAGACAGGTC | |
| 167 | AAUCAACGCUAGACAGGUC | |
| 168 | GACCTGTCTAGCGTTGATT | |
| 169 | GACCUGUCUAGCGUUGAUU | |
| 170 | AATCAACGCTAGACAGGTCTTTAAAAAAAAAAAAAAAAAAAAAAAAAAAAAA | |
| 171 | AAUCAACGCUAGACAGGUCAACCCTTTAAAAAAAAAAAAAAAAAAAAAAAAAAAAAA | |
| 172 | UCAACGCUAGACAGGUCAATTTAAAAAAAAAAAAAAAAAAAAAAAAAAAAAA | |
| 173 | tacttggttgatcctgccaaggaagcacacttaggtcatagattaagccatgcaagtg | T vaginalis |
| | ttagttcaggtaacgaaactgcgaatagctcattaatacgctcagaatctatttggcg | 16S |
| | gcgaccaacaggtcttaaatggatagcagcagcaactctggtgctaatacatgcgatt | |
| | gtttctccagatgtgaattatggaggaaaagttgacctcatcagaggcacgccattcg | |
| | actgagtgacctatcagcttgtacttagggtctttacctaggtaggctatcacgggta | |
| | acgggcggttaccgtcggactgccggagaaggcgcctgagagatagcgactatatcca | |
| | cgggtagcagcaggcgcgaaactttcccactcgagactttcggaggaggtaatgacca | |
| | gttccattggtgccttttggtactgtggataggggtacggttttccaccgtaccgaaa | |
| | cctagcagagggccagtctggtgccagcagctgcggtaattccagctctgcgagtttg | |
| | ctccatattgttgcagttaaaacgccgtagtctgaattggccagcaatggtcgtacgt | |
| | atttttacgttcactgtgaacaaatcaggacgcttagagtatggccacatgaatgact | |
| | cagcgcagtatgaagtctttgttttcttccgaaaacaagctcaatgagagccatcggg | |
| | ggtagatctatctcatgacgagtggtggaatactttgactcatgagagagaagctgag | |
| | gcgaaggcgtctacctagagggtttctgtcgatcaagggcgagagtaggagtatccaa | |
| | caggattagagaccctggtagttcctaccttaaacgatgccgacaggagtttgtcatt | |
| | tgttaatggcagaatctttggagaaatcatagttcttgggctctgggggaactacgac | |
| | cgcaaggctgaaacttgaaggaattgacggaagggcacaccaggggtggagcctgtgg | |
| | cttaatttgaatcaacacggggaaacttaccaggaccagatgttttttatgactgaca | |
| | ggcttcgggtctttcaggatattgtttttggtggtgcatggccgttggtggtgcgtgg | |
| | gttgacctgtctagcgttgattcagctaacgagcgagattatcgccaattatttactt | |
| | tgccgaagtccttcggttaaagttctaattgggactccctgcgattttagcaggtgga | |
| | agagggtagcaataacaggtccgtgatgccctttagatgctctgggctgcacgcgtgc | |
| | tacaatgttaggatcaataggactgcgagcctgagagggtgcgctactcttataatcc | |
| | ctaacgtagttgggattgacgtttgtaatcagcgtcatgaaccaggaatcctcgtaaa | |
| | tgtgtgtcaacaacgcacgttgaatacgtccctgccctttgtacacaccgcccgtcgc | |
| | tcctaccgattggatgactcggtgaaatcaccggatgcttacgagcagaaagtgatta | |
| | aatcacgttatctagaggaaggagaagtcgtaacaaggtaacggtaggtgaacctgcc | |
| | gttggatc | |
| 174 | attgacggaagggcacaccaggggtggagcctgtggcttaatttgaatcaacacgggg | T vaginalis |
| | aaacttaccaggaccagatgttttttatgactgacaggcttcgggtctttcaggatat | 16S target |
| | tgtttttggtggtgcatggccgttggtggtgcgtgggttgacctgtctagcgttgatt | sequence |
| | cagctaacgagcgagattatcgccaattatttactttgccgaagtccttcggttaaag | |
| | ttctaattgggactccctgcgattttagcaggtggaagagggtagcaataacaggtcc | |
| | gtgatgccctttagatgctctgggctgcacgcgtgctacaatgttaggatcaatagga | |
| | ctgcgagcctgagagggtgcgctactcttataatccctaacgtagttgggattgacgt | |
| | ttgtaatcagcgtcatgaaccaggaatcctcgtaaatgtgtgtcaacaacgcacgttg | |
| | aatacgtccctgccctttgtacacaccgcccgtcgctcctaccgattggatgactcgg | |
| | tgaaatcaccggatgcttacgagcagaa | |
| 175 | gcttcgggtctttcaggatattgtttttggtggtgcatggccgttggtggtgcgtggg | T vaginalis |
| | ttgacctgtctagcgttgattcagctaacgagcgagattatcgccaattatttacttt | 16S target |
| | gccgaagtccttcggttaaagttctaattgggactccctgcgattttagcaggtggaa | sequence |
| | gagggtagcaataacaggtccgtgatgccctttagatgctctgggctgcacgcgtgct | |
| | acaatgttaggatcaataggactgcgagcctgagagggtgcgctactcttataatccc | |
| | taacgtagttgggattgacgtttgtaatcagcgtcatgaa | |
| 176 | ttactttgccgaagtccttcggttaaagttctaattgggactccctgcgattttagca | |
| | ggtggaagagggtagcaataacaggtccgtgatgccctttagatgctctgggctgcac | |
| | gcgtgctacaatgttaggatcaataggactgcgagcctgagagggtgcgctactctta | |
| | taatccctaacgtagttgggattgacgtttgtaatcagcgtcatgaa | |
| 177 | ggcttcgggtctttcaggatattgtttttggtggtgcatggccgttggtggtgcgtgg | |
| | gttgacctgtctagcgttgattcagctaacgagcgagattatcgccaattatttactt | |
| | tgccgaagtccttcggttaaagttctaattgggactccctgcgattttagcaggtgga | |
| | agagggtagcaataacaggtccgtgatgccctttagatgctctgg | |
| 178 | ctaattgggactccctgcgattttagcaggtggaagagggtagcaataacaggtccgt | |
| | gatgccctttagatgctctgggctgcacgcgtgctacaatgttaggatcaataggact | |
| | gcgagcctgagagggt | |

*(L) is an optional linker.
The linker can be a nucleic acid linker or a non-nucleic acid linker.
Linkers include, but are not limited to, C1-C16, C₁, C₂, C₃, C₄, C₅, C₆, C₇, C₈, C₉, C₁₀, C₁₁, C₁₂, C₁₃, C₁₄, C₁₅, or C₁₆, PEG, or other suitable linker.

E. Compositions and Kits

The present disclosure provides oligomers, compositions, and kits, useful for amplifying, detecting, and/or quantifying T. vaginalis in a sample. The oligomers, compositions, and kits can be used in uniplex or multiplex multiphase amplification methods.

Reaction mixtures for determining the presence or absence of a T. vaginalis target nucleic acid or quantifying the amount thereof in a sample are described. Various reaction mixtures, include, but not limited to, Target capture (TCR) mixtures, Amplification (AMP) mixtures, promoter primer (PRO) mixtures, and enzyme (ENZ) mixtures. In accordance with the present disclosure the mixture independently comprise one or more of: promoter primer (e.g., T7 primer), non-promoter primer (NT7 oligonucleotide), TCO, detection oligonucleotide, reverse transcriptase, RNA polymerase, dNTPs, NTPs, buffers, salts, and combinations thereof, as described herein for amplification and/or detection of a T. vaginalis target nucleic acid in a sample. In some embodiments, any oligonucleotide combination described herein can be provided in a kit. A composition, kit and/or reaction mixture may further include a number of optional components. In some embodiments, a kit includes one or more test sample components, in which a T. vaginalis target nucleic acid may or may not be present. In some embodiments, a kit includes one or more control oligonucleotides, including, but not limited to, control TCO, control promoter primer, control non-promoter primer, control detection oligonucleotide, and combinations thereof. A kit may include oligonucleotides for amplification and detection of T. vaginalis, or it may oligonucleotides for amplification and detection T. vaginalis and one or more other organisms, including, but not limited to Candida species.

In some embodiments, a composition or kit comprises a detection oligonucleotide that comprises one or more detection oligonucleotides. The detection oligonucleotides independently comprise fluorescent label(s) and quencher(s). In some embodiments, a composition or kit comprises one or more Torch detection oligonucleotides. In some embodiments, a composition or kit comprises two or more Torch detection oligonucleotides. The two or more Torch oligonucleotides can detect amplification products from different organisms and be detectable in different channels.

In some embodiments, a kit, composition, or reaction mixture(s) additionally contains one or more of: DNA polymerase, deoxyribonucleotides, positive control nucleic acid, negative control nucleic acid, control nucleic acid, dNTPs (e.g. dATP, dTTP, dGTP, and dCTP), NTPs (e.g. ATP, UTP, GTP, and CTP), Cl, MgCl₂, potassium acetate, buffer, BSA, sucrose, trehalose, DMSO, betaine, formamide, glycerol, polyethylene glycol, non-ionic detergents, ammonium ions, EDTA, and other reagents or buffers suitable for isothermal amplification and/or detection. The DNA polymerase can be, but is not limited to, reverse transcriptase. The buffer can be, but is not limited to, Tris-HCl and Tris-acetate. The nonionic detergent can be, but is not limited to, Tween-20 and Triton X-100.

In some embodiments, the described primers and detection oligonucleotides for T. vaginalis have a shelf-life of at least 3 months, at least 6 months, at least 9 months, at least 12 months, at least 15 months, at least 18 months, or at least 24 months from date of manufacture.

Any method disclosed herein is also to be understood as a disclosure of corresponding uses of materials involved in the method directed to the purpose of the method. Any of the oligonucleotides comprising T. vaginalis sequence and any combinations (e.g., kits and compositions) comprising such an oligonucleotide are to be understood as also disclosed for use in detecting and/or quantifying T. vaginalis or in amplifying a T. vaginalis nucleic acid sequence, and for use in the preparation of a composition for detecting and/or quantifying T. vaginalis, or in amplifying a T. vaginalis nucleic acid sequence.

In some embodiments, a kit further includes a set of instructions for practicing methods in accordance with the present disclosure, where the instructions may be associated with a package insert and/or the packaging of the kit or the components thereof.

Embodiments of the compositions and methods described herein may be further understood by the examples that follow. Method steps used in the examples have been described herein and the following information describes typical reagents and conditions used in the methods with more particularity. Other reagents and conditions may be used that will not substantially affecting the process or results so long as guidance provided in the description above is followed. Moreover, the disclosed methods and compositions may be performed manually or in a system that performs one or more steps (e.g., pipetting, mixing, incubation, and the like) in an automated device or used in any type of known device (e.g., test tubes, multi-tube unit devices, multi-well devices such as 96-well microtiter plates, and the like).

### EXAMPLES

Exemplary reagents used in the methods described in the examples include the following.

“Sample Transport Medium” or “STM” is a phosphate-buffered solution (pH 6.7) that included EDTA, EGTA, and lithium lauryl sulfate (LLS).

“Target Capture Reagent” or “TCR” is a HEPES-buffered solution (pH 6.4) that included lithium chloride and EDTA, together with 250 μg/ml of magnetic particles (1 micron SERA-MAGTM MG-CM particles, Seradyn, Inc. Indianapolis, Ind.) with (dT)14 oligonucleotides covalently bound thereto.

“Target Capture Wash Solution” or “TC Wash Solution” is a HEPES-buffered solution (pH 7.5) that included sodium chloride, EDTA, 0.3% (v/v) absolute ethanol, 0.02% (w/v) methyl paraben, 0.01% (w/v) propyl paraben, and 0.1% (w/v) sodium lauryl sulfate.

“Amplification Reagent” or “AR” is a HEPES-buffered solution (pH 7.7) that included magnesium chloride, potassium chloride, four deoxyribonucleotide triphosphates (dATP, dCTP, dGTP, and dTTP), four ribonucleotide triphosphates (rATP), rCTP, rGTP, and rUTP). Primers and/or probes may be added to the reaction mixture in the amplification reagent, or may be added separate from the reagent (primerless amplification reagent).

“Enzyme Reagents” or “ENZ”, as used in amplification or pre-amplification reaction mixtures, are HEPES-buffered solutions (pH 7.0) that include MMLV reverse transcriptase (RT), T7 RNA polymerase, salts and cofactors.

### Example A

### Multi-Phase Amplification/Detection

A T7 primer is hybridized to the target sequence during target capture, followed by removal of excess T7 primer.

During the first phase, a NT7 primer is introduced along with all of the requisite amplification, detection and enzyme reagents, with the exception of additional T7 primer. In the presence of reverse transcriptase, the T7 primer hybridized to the captured target is extended, creating a cDNA copy, and the target RNA template is degraded by the reverse transcriptase's RNase H activity. The NT7 primer subsequently hybridizes to the cDNA and is extended, filling in the promoter region of the T7 primer and creating an active, double-stranded DNA template. T7 polymerase then produces multiple RNA transcripts from the template. The NT7 primer subsequently hybridized to the RNA transcripts and is extended, producing promoterless cDNA copies of the target RNA template. The RNA strands are degraded by RNase activity of the reverse transcriptase. Because no free T7 primer is available in the phase 1 amplification mixture, the reaction does not proceed further. The second phase is then started with the addition of T7 primer and optionally detection oligonucleotide, thus initiating exponential amplification of the cDNA pool produced in phase 1.

For multiplex amplification and detection one or more of each of the TCO, T7 primer, NT7 primer and Torch oligonucleotides is used. The oligonucleotides may amplify different sequence in the in the same target nucleic acid or sequences in different target nucleic acids, or a combination thereof. The different target nucleic acids may be from the same or different organisms.

Plate Setup:

In some embodiments, four different plates are set up for use on two automated KingFisher devices.

1. Plate 1 (TCR plate) contains the lysed sample. Target Capture Reagent (100 μL) is added to this plate. The TCO and T7 primer hybridize to target nucleic acid (400 μL sample). The TCO:target nucleic acid:T7 primer (pre-amplification hybrid) are captured using a magnetic bead (capture probe on solid support) using a magnet.

2. Plate 2 is a deep-well plate and holds 500 μL/well APTIMA wash buffer. The Aptima wash buffer contains detergent and alcohol used to wash any excess proteins and lipids leftover from cell lysis.

3. Plate 3 contains 200 μL/well APTIMA wash buffer and is used to provide a second wash of the pre-amplification hybrid.

4. Plate 4 contains 50 μL/well AMP reagent. In some embodiments, the AMP reagent contains buffer, salt, dNTPs, NTPs and one or more nonT7 primers.

Target Capture and isolation: TCO(s) and T7 primer(s) are added to a sample containing (or suspected of containing) the target nucleic acid. T7 primer is added at a ratio of approximately 1 T7 primer to 1 target nucleic acid. TCO and T7 primer are incubated with the target nucleic acid for a period of time to allow hybridization of the TCO and T7 primer to the target nucleic acid. The pre-amplification hybrid is then purified, removing excess or non-hybridized T7 primer. The pre-amplification hybrid is then isolated using magnetic particles having a poly(dT) binding partner for the TCO.
1. Plate 1 (TCR plate) is placed into a heat block and heated to 62° C. for 30 min. followed by room temperature for 20 min-2 h. In some embodiments, the TCR plate is covered with a 65° C. lid to prevent condensation from forming on the tops of the wells. The captured pre-amplification hybrid is then transferred to Plate 2.
2. After the first wash (about 10 min), a deep well comb/magnet cover as added to the Plate 2 to capture the pre-amplification hybrid. The captured pre-amplification hybrid is transferred to Plate 3.
3. After the second wash, a small comb (magnet cover) is added to Plate 3 to capture the pre-amplification hybrid. The washed pre-amplification hybrid is captured and transferred to Plate 4. The 4th plate is transferred to a thermal cycler for real-time isothermal amplification and detection.

Multiphase Transcription Mediated Amplification and Real Time detection. First Phase Amplification: NT7 primer(s), enzymes, dNTPs and NTPs (AMP mixture) are present with the purified target nucleic acid containing the pre-amplification hybrid. The mixture is incubated for a period of time to allow formation of a first amplification product.
1. Incubate AMP plate, containing NT7 primer and purified target nucleic acid with hybridized T7 primer, at 44° for 5 minutes.
2. Add 25 μL of ENZ mix, containing Reverse transcriptase, T7 RNA polymerase, dNTPs, and NTPs, to each well of the plate, seal and mix 1 min 1400 rpm; incubate 5 minutes at 44° C. on a thermal cycler.
   Second Phase Amplification: T7 primer is added to the first amplification product and incubated for a period of time to allow formation of a second amplification product.
3. Add 25 μL PRO mixture to each well, seal, and mix 1 min 1400 rpm. In some embodiments, the PRO mixture contains buffer, salt, surfactant, dNTPs, NTPs, one or more T7 primers and Torch probes.
4. Run reaction program: 120 cycles of 30 seconds at 43° C. with label detection (collection) at the end of each cycle.

Detection: Amplification of the target nucleic acid sequence is detected in real time by recording fluorescent signal from the detection oligonucleotide at regular intervals.

### Example 1

### T. vaginalis Biphasic Real Time TMA Oligo Screen

Multiphase amplification was performed as described above using the following conditions.

TABLE 1-1
TCR mixture: TCO final concentration = 15 pmol/reaction.

| | TCO | Stock conc. | μL TC | μL TC | |
|---|---|---|---|---|---|
| | SEQ ID NO. | pmol/μL | oligo | reagent | N rxns |
| TC1 | 2 | 61.85 | 8.49 | 3491.5 | 35 |

TABLE 1-2
AMP mixture: NT7 primer final reaction
concentration = 2.67 pmol/reaction.

| | NT7 primer | Stock conc. | μL | μL AMP | N |
|---|---|---|---|---|---|
| | SEQ ID NO. | pmol/μL* | NT7 primer | reagent | reactions |
| AMP1 | 15 | 23.777 | 4.49 | 1995.5 | 40 |

TABLE 1-3
PRO mixture: T7 primer final reaction concentration = 2.67 pmol/reaction and Torch
oligo final reaction concentration = 15 pmol/reaction.

| | | T7 primer | Torch | Torch oligo | μL | μL | | |
|---|---|---|---|---|---|---|---|---|
| | T7 primer | stock | SEQ ID | stock | T7 | Torch | μL AMP | N |
| | SEQ ID NO. | pmol/μL | NO. | pmol/μL | oligo | oligo | reagent | rxns |
| PRO1 | 6 | 9.1 | 25 | 93.32 | 4.11 | 2.25 | 343.64 | 14 |
| PRO2 | 7 | 6.3 | 25 | 93.32 | 5.93 | 2.25 | 341.82 | 14 |
| PRO3 | 8 | 5.97 | 25 | 93.32 | 6.26 | 2.25 | 341.49 | 14 |
| PRO4 | 6 | 9.1 | 26 | 95.18 | 4.11 | 2.21 | 343.69 | 14 |

TABLE 1-4
Reaction mixtures: volume per reaction

| | μL/reaction |
|---|---|
| TCR mixture | 100 |
| AMP mixture | 50 |
| ENZ mixture | 25 |
| PRO mixture | 25 |

TABLE 1-5
Combinations: TCO was SEQ ID NO: 2; Reactions were run with
0, 1.00 × 10², 1.00 × 10⁴, and 1.00 × 10⁵ target cells per reaction.

| | NT7 primer | T7 primer | Torch |
|---|---|---|---|
| System | SEQ ID NO. | SEQ ID NO. | ID NO. |
| PRO1 | 15 | 6 | 25 |
| PRO2 | 15 | 7 | 25 |
| PRO3 | 15 | 8 | 25 |
| PRO4 | 15 | 6 | 26 |

TABLE 1-6
Oligos.

| Oligo | | | | |
|---|---|---|---|---|
| SEQ ID NO. | Type | Length | OD/mL | pmol/μL |
| 25 | Torch | 30 | 23.1 | 93.32 |
| 26 | Torch | 30 | 23.56 | 95.18 |
| 8 | T7 | 55 | 27.1 | 59.72 |
| 6 | T7 | 49 | 36.79 | 91.00 |
| 7 | T7 | 54 | 28.07 | 63.00 |
| 15 | NT7 | 22 | 43.16 | 237.77 |
| 2 | TCO | 60 | 30.62 | 61.85 |

Results: None of the combinations yielded sufficiently strong curves to enable amplification and/or detection of T. vaginalis.

### Example 2

### T. vaginalis Biphasic Real Time TMA Oligo Screen

Screen alternate target captures and titrate the T. vaginalis T7 primer to see if assay performance improves. Multiphase amplification was performed as described above using the following conditions.

TABLE 2-1
TCR mixture: TCR oligo final concentration was 15 pmol/reaction.

| | TCO | Stock cone. | μL | μL TC | N |
|---|---|---|---|---|---|
| | SEQ ID NO. | pmol/μL | TC oligo | reagent | reactions |
| TC1 | 2 | 61.85 | 4.85 | 1995.1 | 20 |
| TC2 | 3 | 54.65 | 4.12 | 1495.9 | 15 |
| TC3 | 1 | 75.14 | 2.99 | 1497.0 | 15 |

TABLE 2-2
AMP mixture: NT7 primer final reaction
concentration was 2.67 pmol/reaction.

| | NT7 primer | Stock cone. | μL | μL AMP | N |
|---|---|---|---|---|---|
| | SEQ ID NO. | pmol/μL* | NT7 primer | reagent | reactions |
| AMP1 | 15 | 23.777 | 4.49 | 1995.5 | 40 |

TABLE 2-3
PRO mixture: Torch oligo final reaction concentration was 15 pmol/reaction.

| | | pmol/ | T7 primer | | μL | μL | | |
|---|---|---|---|---|---|---|---|---|
| | T7 primer | rxn | stock | Torch | T7 | Torch | μL AMP | N |
| | SEQ ID NO. | T7* | pmol/μL | SEQ ID NO. | oligo | oligo | reagent | rxns |
| PRO1 | 6 | 2.67 | 9.1 | 26 | 5.87 | 3.15 | 490.98 | 20 |
| PRO2 | 6 | 5 | 9.1 | 26 | 8.24 | 2.36 | 364.39 | 15 |
| PRO3 | 6 | 7.5 | 9.1 | 26 | 12.36 | 2.36 | 360.27 | 15 |
| PRO4 | 6 | 10 | 9.1 | 26 | 15.48 | 2.36 | 356.15 | 15 |

TABLE 2-4
Reaction mixtures: volume per reaction

| | μL/reaction |
|---|---|
| AMP mixture | 50 |
| PRO mixture | 25 |
| TCR mixture | 100 |
| ENZ mixture | 25 |

TABLE 2-5
Combinations: T7 primer = SEQ ID NO: 6; NT7
primer = SEQ ID NO: 15; Torch oligo =
SEQ ID NO: 26. Reactions were run with 0, 1.00 × 10³ and
1.00 × 10⁵ target cells per reaction.

| | NT7 primer | NT7 primer conc. | TCO oligo |
|---|---|---|---|
| System | SEQ ID NO. | pmol/rxn | SEQ ID NO. |
| PRO1/TC1 | 15 | 2.67 | 2 |
| PRO2/TC1 | 15 | 5 | 2 |
| PRO3/TC1 | 15 | 7.5 | 2 |
| PRO4/TC1 | 15 | 10 | 2 |
| PRO1/TC2 | 15 | 2.67 | 3 |
| PRO1/TC3 | 15 | 2.67 | 1 |

TABLE 2-6
Oligos

| SEQ ID NO. | Type | Length | OD/mL | pmol/uL |
|---|---|---|---|---|
| 25 | Torch | 30 | 23.1 | 93.32 |
| 26 | Torch | 30 | 23.56 | 95.18 |
| 8 | T7 | 55 | 27.0 | 59.72 |
| 15 | nT7 | 22 | 43.16 | 237.77 |
| 2 | TCO | 60 | 30.62 | 61.85 |
| 8 | T7 | 49 | 36.79 | 91.00 |
| 7 | T7 | 54 | 28.07 | 63.00 |
| 3 | TCO | 57 | 25.7 | 54.65 |
| 1 | TCO | 55 | 34.1 | 75.14 |
| 3 | TCO | 57 | 25.23 | 53.65 |
| 1 | TCO | 55 | 38.05 | 83.85 |
| 6 | T7 | 49 | 36.79 | 91.00 |
| 4 | T7 | 52 | 32.32 | 75.33 |
| 5 | T7 | 46 | 42.06 | 110.82 |

Results: None of the combinations yielded sufficiently strong curves to enable amplification and/or detection of T. vaginalis.

### Example 3

### T. vaginalis Biphasic Real Time TMA Oligo Screen

Multiphase amplification was performed as described above using the following conditions.

TABLE 3-1
TCR mixture: TCO stock concentration = 61.85 pmol/μL;
TCO final concentration = 15 pmol/reaction.

| | TCO | | μL TC | N |
|---|---|---|---|---|
| | SEQ ID NO. | μL stock | reagent | rxns |
| TC1 | 2 | 8.49 | 3491.5 | 35 |

TABLE 3-2
AMP mixture: NT7 primer final
reaction concentration = 10 pmol/reaction.

| | NT7 | | | | | |
|---|---|---|---|---|---|---|
| | primer | | Stock | μL | μL | N |
| | SEQ | | conc. | NT7 | AMP | reac- |
| | ID NO. | Region | pmol/μL* | primer | reagent | tions |
| AMP1 | 14 | 1089 | 50 | 3.00 | 747.0 | 15 |
| AMP2 | 13 | 1089 | 50 | 3.00 | 747.0 | 15 |
| AMP3 | 15 | 1168 | 23.77 | 6.31 | 743.70 | 15 |

TABLE 3-3
PRO mixture: T7 primer final reaction concentration = 10 pmol/reaction
and Torch oligo final reaction concentration = 15 pmol/reaction.

| | T7 | | | | | |
|---|---|---|---|---|---|---|
| | primer | Torch | μL | μL | | |
| | SEQ ID | SEQ ID | T7 | Torch | μL AMP | N |
| | NO. | NO. | oligo | oligo | reagent | rxns |
| PRO1 | 4 | 20 | 2.00 | 3.00 | 245.00 | 10 |
| PRO2 | 5 | 20 | 2.00 | 3.00 | 245.00 | 10 |
| PRO3 | 4 | 21 | 2.00 | 3.00 | 245.00 | 10 |
| PRO4 | 5 | 21 | 2.00 | 3.00 | 245.00 | 10 |
| PRO5 | 9 | 23 | 2.00 | 3.00 | 245.00 | 10 |
| PRO6 | 10 | 23 | 2.00 | 3.00 | 245.00 | 10 |
| PRO7 | 6 | 22 | 2.00 | 3.00 | 245.00 | 10 |
| PRO8 | 6 | 25 | 2.00 | 3.00 | 245.00 | 10 |
| PRO9 | 6 | 26 | 2.00 | 3.00 | 245.00 | 10 |

TABLE 3-4
Reaction mixtures: volume per reaction

| | μL/ |
|---|---|
| | reaction |
| AMP mixture | 50 |
| PRO mixture | 25 |
| TCR mixture | 100 |
| ENZ mixture | 25 |

TABLE 3-5
Combinations: TCO = SEQ ID NO: 2.

| | NT7 primer | T7 primer | |
|---|---|---|---|
| | SEQ ID NO. | SEQ ID NO. | Torch SEQ ID NO. |
| AMP1/PR01 | 14 | 4 | 20 |
| AMP2/PR01 | 13 | 4 | 20 |
| AMP1/PR02 | 14 | 5 | 20 |
| AMP2/PR02 | 13 | 5 | 20 |
| AMP1/PR03 | 14 | 4 | 21 |
| AMP2/PR03 | 13 | 4 | 21 |
| AMP1/PR04 | 14 | 5 | 21 |
| AMP2/PR04 | 13 | 5 | 21 |
| AMP3/PR05 | 15 | 9 | 23 |
| AMP3/PR06 | 15 | 10 | 23 |
| AMP3/PR07 | 15 | 6 | 22 |
| AMP3/PR08 | 15 | 6 | 25 |
| AMP3/PR09 | 15 | 6 | 26 |

TABLE 3-6
Oligos.

| Oligo Type | SEQ ID NO. | Length | OD/mL | pmol/μL |
|---|---|---|---|---|
| nT7 | 14 | 20 | 36.64 | 222.04 |
| nT7 | 13 | 25 | 36.44 | 176.66 |
| nT7 | 15 | 22 | 43.16 | 237.77 |
| Torch | 20 | 19 | 31.53 | 201.13 |
| Torch | 21 | 17 | 25.86 | 184.37 |
| Torch | 22 | 28 | 29.8 | 128.99 |
| Torch | 22 | 27 | 28.28 | 126.95 |
| Torch | 25 | 30 | 23.1 | 93.32 |
| Torch | 26 | 30 | 23.56 | 95.18 |
| T7 | 9 | 50 | 33.74 | 81.79 |
| T7 | 10 | 45 | 31.1 | 83.76 |
| T7 | 6 | 49 | 36.79 | 91.00 |
| T7 | 4 | 52 | 32.32 | 75.33 |
| T7 | 5 | 46 | 42.06 | 110.82 |

Results: AMP1/PRO1, AMP1/PRO3, AMP2/PRO3, and AMP3/PRO5 gave good amplification and/or detection of T. vaginalis.

TABLE 3-7
Results (TC oligo = 809)

| | NT7 primer | Torch | T7 primer | Detection | |
|---|---|---|---|---|---|
| Combination | SEQID NO. | SEQ ID NO. | SEQ ID NO. | Curve | RFU |
| AMPl/PR0l | 14 | 20 | 4 | ++ | 11000 |
| AMP1/PR02 | 14 | 20 | 5 | − | |
| AMP1/PR03 | 14 | 21 | 4 | ++ | 7000 |
| AMP1/PR04 | 14 | 21 | 5 | − | |
| AMP2/PR01 | 13 | 20 | 4 | + | 12000 Fanning |
| AMP2/PR02 | 13 | 20 | 5 | − | |
| AMP2/PR03 | 13 | 21 | 4 | ++ | 7500 |
| AMP2/PR04 | 13 | 21 | 5 | − | |
| AMP3/PR05 | 15 | 23 | 9 | ++ | 27000 slight fanning |
| AMP3/PR06 | 15 | 23 | 10 | − | |
| AMP3/PR07 | 15 | 22 | 6 | − | 25000 separating reps |
| AMP3/PR08 | 15 | 25 | 6 | − | Linear |
| AMP3/PR09 | 15 | 26 | 6 | − | Linear |

### Example 4

### T. vaginalis Biphasic Real Time TMA Oligo Screen

Multiphase amplification was performed as described above using the following conditions.

TABLE 4-1
TCR mixture:

| | SEQ ID NO. | μL stock | μL TC reagent | N reactions |
|---|---|---|---|---|
| TC1 | 3 | 9.61 | 3490.4 | 35 |

TC1 oligo:
stock concentration = 54.65 pmol/μL;
final concentration = 15 pmol/reaction
TCR mixture: 100 μL/reaction

TABLE 4-2
AMP mixture. NT7 primer = 10 pmol/reaction

| | SEQ ID NO. | μL T7 primer | μL AMP reagent | N reactions |
|---|---|---|---|---|
| AMP4 | 19 | 3.00 | 747.0 | 15 |
| AMP5 | 16 | 3.00 | 747.0 | 15 |
| AMP6 | 17 | 3.00 | 747.0 | 15 |
| AMP7 | 18 | 3.00 | 747.0 | 15 |

TABLE 4-3
PRO mixture. T7 primer = 10 pmol/reaction;
Torch = 15 pmol/reaction

| | T7 | Torch | | | | |
|---|---|---|---|---|---|---|
| | primer | SEQ | μL | μL | μL | |
| | SEQ | ID | T7 | Torch | AMP | N |
| | ID NO. | NO. | oligo | oligo | reagent | rxns |
| PRO10 | 11 | 27 | 3.00 | 4.5 | 367.5 | 15 |
| PRO11 | 11 | 27 | 3.00 | 4.5 | 367.5 | 15 |
| PRO12 | 11 | 28 | 3.00 | 4.5 | 367.5 | 15 |

TABLE 4-4
Reaction mixtures: volume per reaction

| | μL/ reaction |
|---|---|
| AMP mixture | 50 |
| PRO mixture | 25 |
| TCR mixture | 100 |
| ENZ mixture | 25 |

TABLE 4-5
Oligos

| Oligo | | | | |
|---|---|---|---|---|
| SEQ ID NO. | Type | Length | OD/mL | pmol/μL |
| 3 | TCO | | | |
| 19 | NT7 | 24 | 27.08 | 136.75 |
| 16 | NT7 | 21 | 37.62 | 217.12 |
| 17 | NT7 | 22 | 34.00 | 187.31 |
| 18 | NT7 | 23 | 35.31 | 186.07 |
| 11 | T7 | 49 | 31.86 | 78.80 |
| 12 | T7 | 50 | 37.98 | 92.06 |
| 27 | Torch | 27 | 33.00 | 148.13 |
| 28 | Torch | 26 | 21.10 | 98.82 |

TABLE 4-6
Combinations:
TCO = SEQ ID NO: 3, T7 primer = SEQ ID NO: 11

| | NT7 primer | Torch |
|---|---|---|
| | SEQ ID NO. | SEQ ID NO. |
| PRO10/AMP4 | 19 | 27 |
| PRO11/AMP4 | 19 | 27 |
| PRO12/AMP4 | 19 | 28 |
| PRO10/AMP5 | 16 | 27 |
| PRO11/AMP5 | 16 | 27 |
| PRO12/AMP5 | 16 | 28 |
| PRO10/AMP6 | 17 | 27 |
| PRO11/AMP6 | 17 | 27 |
| PRO12/AMP6 | 17 | 28 |
| PRO10/AMP7 | 18 | 27 |
| PRO11/AMP7 | 18 | 27 |
| PRO12/AMP7 | 18 | 28 |

While some of the systems showed amplification, none of the systems produced strong curves. While the indicated oligos may be candidates for viable systems, none of the combinations performed well in amplification/detection of T. vaginalis.

### Example 5

### T. tenax Cross Reactivity with T. vaginalis Amplification System

Multiphase amplification was performed as described above using the following conditions.

TABLE 5-1
TCR mixture:
TCO final concentration = 15 pmol/reaction. (70 reactions)

| | oligo | Oligo stock | | Oligo |
|---|---|---|---|---|
| reagent | SEQ ID NO. | concentration | μL stock | pmol/rxn |
| TCO | 3 | 53.65 | 19.57 | 15 |
| T7 primer | 11 | 50.00 | 21.00 | 15 |
| TC reagent | | | 6959.4 | |

TABLE 5-2
AMP mixture: NT7 primer final reaction concentration = 10 pmol/reaction.

| | NT7 primer | Stock cone. | μL | μL | |
|---|---|---|---|---|---|
| | SEQ ID NO. | pmol/μL | NT7 primer | AMP reagent | N reactions |
| AMP1 | 15 | 23.77 | 29.45 | 3470.6 | 70 |

TABLE 5-3
PRO mixture: T7 primer final reaction concentration = 10 pmol/reaction and Torch
oligo final reaction concentration = 15 pmol/reaction.

| | T7 primer | Torch | μL | Torch | μL | μL AMP | N |
|---|---|---|---|---|---|---|---|
| | SEQ ID NO. | SEQ ID NO. | T7 oligo | pmol/rxn | Torch oligo | reagent | rxns |
| PRO1 | 11 | 23 | 3.80 | 15 | 5.7 | 465.5 | 19 |
| PRO2 | 11 | 23 | 3.80 | 10 | 3.8 | 467.4 | 19 |
| PRO3 | 11 | 23 | 3.80 | 5 | 1.9 | 469.3 | 19 |
| PRO4 | 11 | 64 | 3.80 | 5 | 1.9 | 469.3 | 19 |

TABLE 5-4
Reaction mixtures: volume per reaction

| | μL/reaction |
|---|---|
| AMP mixture | 50 |
| PRO mixture | 25 |
| TCR mixture | 100 |
| ENZ mixture | 25 |

TABLE 5-5
Combinations:
TCO = SEQ ID NO: 3; NT7 primer = SEQ ID NO: 15

| | T. vaginalis | T. tenax | Torch |
|---|---|---|---|
| PRO Mix | cells/reaction | cells/reaction | SEQ ID NO. |
| PR0l | 0 | 0 | 23 |
| PR02 | 0 | 0 | 23 |
| PR03 | 0 | 0 | 23 |
| PR04 | 0 | 0 | 4 |
| PR0l | 0 | 1.00 × 10⁵ | 23 |
| PR02 | 0 | 1.00 × 10⁵ | 23 |
| PR03 | 0 | 1.00 × 10⁵ | 23 |
| PR04 | 0 | 1.00 × 10⁵ | 4 |
| PR0l | 1.00 × 102 | 1.00 × 10⁵ | 23 |
| PR02 | 1.00 × 102 | 1.00 × 10⁵ | 23 |
| PR03 | 1.00 × 102 | 1.00 × 10⁵ | 23 |
| PR04 | 1.00 × 102 | 1.00 × 10⁵ | 4 |
| PR0l | 1.00 × 102 | 0 | 23 |
| PR02 | 1.00 × 102 | 0 | 23 |
| PR03 | 1.00 × 102 | 0 | 23 |
| PR04 | 1.00 × 102 | 0 | 4 |

TABLE 5-6
Oligos.

| SEQ. ID NO. | Type | Length | OD/mL | pmol/μL |
|---|---|---|---|---|
| 23 | Torch | 28 | 29.8 | 128.99 |
| 64 | Torch | 26 | 26.6 | 124.00 |
| 3 | TCO | 57 | 25.7 | 54.65 |
| 11 | T7 | 49 | 31.86 | 78380 |
| 15 | NT7 | 22 | 43.16 | 237.77 |

Results: The presence of 1×10⁵ T. tenax cells/reaction of did not interfere with T. vaginalis detection using the indicated oligonucleotides. T. vaginalis was detected with the same emergence point and reached the same RFU whether or not T. tenax was present. The indicated oligonucleotides detected T. tenax albeit with a substantially slower emergence time (slower ˜8 min. vs. ˜14 min) and a lower RFU (˜22,000 vs. 7300 at 15 pmol Torch). Torch SEQ ID NO: 64 exhibited very low background with T. tenax.

### Example 6

### T. tenax and Pentatrichomonas hominis Cross Reactivity with T. vaginalis Amplification System

Multiphase amplification was performed as described above using the following conditions. N7 oligonucleotide SEQ ID NO: 9, was compared with N7 oligonucleotide SEQ ID NO: 11 and Torch SEQ ID NO: 23 was compared with Torch SEQ ID NO: 64 for specificity of amplifying T. vaginalis vs. T. tenax and P. hominis. Bi-phase amplification reactions were carried out as described utilizing TCO SEQ ID NO: 3 and NT7 primer SEQ ID NO: 15. Torch SEQ ID NO: 23 provided the stronger amplification curves (Table 5-7). N7 oligonucleotide SEQ ID NO: 11 provided less background due to later TTime and lower RFU range (Table 5-8).

TABLE 6-1
Torch comparison. Torches were used as 15 pmol/reaction.

| Condition | Average T-Time | Average RDU Range |
|---|---|---|
| 0 T. Vaginalis/1 × 105 T. tenax | | |
| T. Tena × Torch, SEQ ID. NO: 64 | 18.68 | 1756.2 |
| T. Vaginalis Torch, SEQ ID NO: 24 | 7.2 | 8134.8 |
| 100 T. Vaginalis/O T. tenax | | |
| T. Tena × Torch, SEQ ID. NO: 64 | 7.5 | 16070.4 |
| T. Vaginalis Torch, SEQ ID NO: 24 | 4.00 | 22537.4 |
| 100 T. Vaginalis/1 × 10⁵ T tenax | | |
| T. Tena × Torch, SEQ ID. NO: 64 | 7.68 | 17224.4 |
| T. Vaginalis Torch, SEQ ID NO: 24 | 3.72 | 22310.33 |

TABLE 6-2
N7 oligonucleotide comparison.

| | Average T-Time | Average T-Time | Average RDU |
|---|---|---|---|
| Condition | NonNorm | Norm | Range |
| SEQ ID NO: 9 | | | |
| 0 T. Vaginalis/1 × 10⁵ P. hominis | 14.5 | 9.00 | 5566.13 |
| 1×10² T. Vaginalis/P. hominis | 4.39 | 4.52 | 21729.6 |
| 1×102 T. Vaginalis/l × 10⁵ P. hominis | 4.06 | 4.19 | 23856.53 |
| NTC | 27.03 | 17.28 | 1655.13 |
| SEQ ID NO: 11 | | | |
| 0 T. Vaginalis/1 × 10⁵ P. hominis | 21.82 | 10.73 | 1499.67 |
| 1 × 102 T. Vaginalis/P. hominis | 11.84 | 11.83 | 16591.53 |
| 1 × 102 T. Vaginalis/1 × 10⁵ P. hominis | 4.37 | 4.22 | 18728.40 |
| NTC | 21.19 | −0.03 | 2552.53 |

No cross reactivity was observed between the closely related non-target species P. hominis and T. vaginalis.

Performance of T. vaginalis T7 primers SEQ ID NO: 9 and SEQ ID NO: 11 with SEQ ID NO: 24 was confirmed in the multiplex format with all assay oligonucleotides including Candida species group and C. glabrata. T7 primer SEQ ID NO: 11 had lower T. tenax background compared to SEQ ID NO: 9 in the CV/TV multiplex assay.

T7 primer SEQ ID NO: 11 had lower T. tenax background by RFU range (5,992 vs. 4,921) and later emerging T-time (14.88 vs. 6.30) compared to SEQ ID NO: 9 using the same torch in a CV/TV multiplex amplification assay (Table 5-9).

TABLE 6-3
N7 oligonucleotide comparison.

| | Average | Average |
|---|---|---|
| | T-Time | RDU |
| Condition | Norm | Range |
| SEQ ID NO: 9 | | |
| 100 T. Vaginalis/0 T. tenax | 6.93 | 17788.50 |
| 100 T. Vaginalis/1 × 10⁵ T. tenax | 6.13 | 17547.80 |
| 0 T. Vaginalis/1 × 10⁵ T. tenax | 6.30 | 5991.90 |
| NTC | 0.43 | 1601.00 |
| SEQ ID NO: 11 | | |
| 100 T. Vaginalis/0 T. tenax | 7.74 | 20642.50 |
| 100 T. Vaginalis/1 × 10⁵ T. tenax | 8.07 | 19182.30 |
| 0 T. Vaginalis/1 × 10⁵ T. tenax | 14.88 | 4920.80 |
| NTC | 0.40 | 1493.00 |

T7 primer SEQ ID NO: 11 had lower T. tenax background by RFU range (5,992 vs. 4,921) and later emerging T-time (14.88 vs. 6.30) compared to SEQ ID NO: 9 using the same torch in a CV/TV multiplex amplification assay (Table 5-9).

### Example 7

### Multiplex Amplification of T. vaginalis and Candida Species

Bi-phase amplification was carried out as described above using following conditions.

TABLE 7-1
TCR mixture: Target Capture Reagent (Aptima TCR) +
Candida and T. vaginalis target capture oligonucleotides.

| TCO | | pmol/ | pmol/ |
|---|---|---|---|
| SEQ ID NO. | μL stock | μL Stock | reaction |
| Aptima TCR Reagent | 4648.2 | | |
| 30 | 4.68 | 50 | 5.00 |
| 29 | 4.68 | 50 | 5.00 |
| 31 | 4.68 | 50 | 5.00 |
| 32 | 1.87 | 25 | 1.00 |
| 33 | 1.87 | 25 | 1.00 |
| 3 | 14.04 | 50 | 15.00 |
| Total volume | 4680 | | |

TABLE 7-2
AMP mixture: AMP reagent + NT7 primers.
Mixtures contain the Candida (Calb, Cgla, and Cpar) NT7
primers and the indicated T. vaginalis NT7 primer.

| | | | pmol/ | pmol/ |
|---|---|---|---|---|
| | SEQ ID NO. | μL | μL stock | reaction |
| All | AMP reagent | 1286.0 | | |
| All | 35 | 3.12 | 25 | 3.00 |
| All | 36 | 3.12 | 25 | 3.00 |
| All | 34 | 2.60 | 50 | 5.00 |
| AMP1 | 14 | 5.20 | 50 | 10.00 |
| AMP2 | 14 | 5.20 | 50 | 10.00 |
| AMP3 | 13 | 5.20 | 50 | 10.00 |
| AMP4 | 15 | 5.20 | 50 | 10.00 |
| AMP5 | 15 | 5.20 | 50 | 10.00 |
| | Total volume | 1300 | | |

TABLE 7-3
PRO mixture: Pro Reagent + Candida and T. vaginalis T7 and Torch oligonucleotides

| | T7 primer | | pmol/μL | pmol/ | Torch SEQ ID | | pmol/μL | pmol/ |
|---|---|---|---|---|---|---|---|---|
| | SEQ ID NO. | μL | stock | rxn | NO. | μL | stock | rxn |
| All | Pro reagent | 613.6 | | | | | | |
| All | 32 | 2.08 | 50 | 4.00 | 38 (FAM Torch) | 5.20 | 50 | 10.00 |
| All | 33 | 2.60 | 50 | 5.00 | 37 (HEX Torch) | 13.52 | 50 | 26.00 |
| PRO1 | 4 | 5.20 | 50 | 15.00 | 20 | 7.8 | 50 | 15 |
| PRO2 | 4 | 5.20 | 50 | 15.00 | 21 | 7.8 | 50 | 15 |
| PRO3 | 4 | 5.20 | 50 | 15.00 | 21 | 7.8 | 50 | 15 |
| PRO4 | 9 | 5.20 | 50 | 15.00 | 23 | 7.8 | 50 | 15 |
| PRO5 | 6 | 5.20 | 50 | 15.00 | none | | | |

TABLE 7-4
Reaction mixtures: volume per reaction

| | μL/reaction |
|---|---|
| AMP mixture | 50 |
| PRO mixture | 25 |
| TCR mixture | 100 |
| ENZ mixture | 25 |

TABLE 7-5
T. vaginalis Oligonucleotides.

| SEQ ID NO. | Type | Length | OD/mL | pmol/μL |
|---|---|---|---|---|
| 14 | nT7 | 20 | 36.64 | 222.04 |
| 13 | nT7 | 25 | 36.44 | 176.66 |
| 15 | nT7 | 22 | 43.16 | 237.77 |
| 20 | Torch | 19 | 31.53 | 201.13 |
| 21 | Torch | 17 | 25.86 | 184.37 |
| 23 | Torch | 28 | 29.8 | 128.99 |
| 9 | T7 | 50 | 33.74 | 81.79 |
| 6 | T7 | 49 | 36.79 | 91.00 |
| 4 | T7 | 52 | 32.32 | 75.33 |
| 2 | TCO | 60 | 30.62 | 61.85 |
| 3 | TCO | 57 | 25.7 | 54.65 |

TABLE 7-6
Combinations. Reactions contained 0, 1.00 × 10⁴ or 1.00 × 10⁶
C. albicans or C. glabrata target cells per reaction
or 0, 0.5, or 1 cell/reaction T. vaginalis. N = 2.

| | NT7 primer | Torch | T7 primer | TCO | |
|---|---|---|---|---|---|
| System | SEQ ID NO. | SEQ ID NO. | SEQ ID NO. | SEQ ID NO. | Components |
| S1 | 14 | 20 | 4 | 2 | A1/P1 |
| S2 | 14 | 21 | 4 | 2 | A2/P2 |
| S3 | 13 | 21 | 4 | 2 | A3/P3 |
| S4 | 15 | 23 | 9 | 3 | A4/P4 |
| S5 | 15 | | 6 | 3 | A5/P5 |

Results: Both the C. albicans and T. vaginalis Torches were read in the FAM channel.

The S1 T. vaginalis oligos partially inhibited C. albicans amplification when 1×10⁴ cells/reaction C. albicans were present in the reaction but not when 1×10⁶ cells/reaction C. albicans were present in the reaction. None of the 5 T. vaginalis oligo combinations affected amplification of C. albicans when 1×10⁶ cells/reaction C. albicans were present in the reaction. Additionally, none of the 5 T. vaginalis oligo combinations adversely affected amplification of C. glabrata.

At 0.1 T. vaginalis cell/reaction System S4 amplified and detected T. vaginalis. At 1 T. vaginalis cell/reaction Systems S1, S2, and S3 amplified and detected T. vaginalis. Amplification of T. vaginalis was not significantly inhibited by the presence of the Candida oligos.

### Example 8

### Multiplex Assay Optimization

Multiplex multiphase amplification was performed as described above using the following conditions. Multiplex assay were performed using Torches SEQ ID NO: 22 and SEQ ID NO: 23 containing Carboxy-X-Rhodamine (ROX) for detection of T. vaginalis. The T. vaginalis TCO was SEQ ID NO: 3, the NT7 primer was SEQ ID NO: 15, and the T7 primer was SEQ ID NO: 11. The multiplex assay additionally contained oligonucleotides for detection of C. albicans and other Candida species (each detected in the FAM channel) and C. glabrata (detected in the HEX channel). A control Torch was detected in the Cy5.5 channel. Candida oligonucleotides are listed in Table 9-5.

The four targets combined with the competitive control were tested in multiplex format. Titration of oligonucleotide concentrations for the Candida species and C. glabrata channels were performed to find a balance among all amplification systems. A formulation of increased amounts of the Candida species oligonucleotides of the T7 in the TCR and NT7 was tested and verified. Next, the Candida species oligo concentrations were tested with increases to C. glabrata T7 in the TCR and NT7. Both sets of testing showed no inhibition of the other channels.

Optimization of Candida species oligo concentrations saw improvement in FAM channel comparing system 1 original concentrations (6 pmol/rxn SEQ ID NO: 35; 5 pmol/rxn SEQ ID NO: 36) to system 2 increased oligo concentrations.

A second optimization of the C. glabrata amplification system increased oligonucleotide concentrations along with the increased C. albicans oligo concentrations. Faster TTime in HEX channel for C. glabrata was observed without changing the amplification efficiency of Candida species in FAM. The competitive control was also improved with the new C. glabrata oligo concentration increases.

Upon testing of combinations of targets, a noted negative interaction between the amplification of C. glabrata in the presence of high titer T. vaginalis was discovered. A 4-Factor Characterization design strategy was selected with high, mid, and low concentrations of T7 in the TCR and NT7 in AMP for both C. glabrata and T. vaginalis in order to determine which factors had the greatest impact in T-time for each analyte. The high concentration was set as the current concentration. The experiment consisted of 20 runs. It was found that a lower concentration of TV T7 in the TCR allowed for faster amplification of C. glabrata.

Using Torch SEQ ID NO: 23 T. vaginalis was detected at 0.001 cells/mL in the multiplex.

### Example 9

### Analytical Sensitivity

Serial dilutions of culture lysates in Aptima Transport Media (STM) for each Candida species (C. albicans, C. tropicalis, C. dubliniensis, C. parapsilosis and C. glabrata) and T. vaginalis were tested with a CV/TV multiplex assay. For each species, 15 reps of ½ log titrations from 1000 CFU/mL to 30 CFU/mL for C. albicans, C. tropicalis and C. dubliniensis, 300 CFU/mL to 3 CFU/mL for C. parapsilosis, 100 CFU/mL to 10 CFU/mL for C. glabrata, and 0.01 cells/mL to 0.0001 cells/mL for T. vaginalis were run. Multiphase amplification was performed as described using the following conditions.

Percent Positivity, Average TTime, Average RFU Range, and Average T-slope for Candida species are shown in Table 9-7. Candida species was detected in the FAM channel, C. glabrata in the HEX channel, T. vaginalis in the ROX channel, and C. glabrata competitive control Torch in the Cy5.5 channel.

Percent Positivity, Average TTime, Average RFU Range, and Average T-slope for T. vaginalis is shown in Table 9-8. The limit of detection to reach 100% positive signal for T. vaginalis was 0.001 cells/ml.

TABLE 9-1
Target Capture Mix

| | | Oligo | Conc. pmol/ |
|---|---|---|---|
| Target | Class | SEQ ID NO | reaction |
| C. spp Group* | Capture | 30 | 5 |
| C. spp Group | Capture | 29 | 5 |
| C. glabrata | Capture | 31 | 5 |
| T. vaginalis | Capture | 3 | 7.5 |
| C. spp Group | T7 primer | 32 | 5 |
| C. glabrata | T7 primer | 33 | 4.83 |
| T. vaginalis | T7 primer | 11 | 1.88 |

*Candida species group = C. albicans, C. parapsilosis, C. tropicalis, C. dubliniensis

TABLE 9-2
AMP Mix

| | NT7 primer | Conc. pmol/ |
|---|---|---|
| Target | SEQ ID NO | reaction |
| C. spp Group | 35 | 6 |
| C. parapsilosis | 34 | 5 |
| C. glabrata | 36 | 5 |
| T. vaginalis | 15 | 8.08 |

TABLE 9-3
PRO Mix

| | | Oligo | Conc. pmol/ |
|---|---|---|---|
| Target | Class | SEQ ID NO | reaction |
| C. spp Group* | T7 Primer | 32 | 8 |
| C. glabrata | T7 Primer | 33 | 8.58 |
| T. vaginalis | T7 Primer | 11 | 10.75 |
| C. spp Group | Torch | 38 | 10 |
| C. glabrata | Torch | 37 | 15 |
| T. vaginalis | Torch | 24 | 12.5 |
| Control | Torch | 63 | 15 |

*Candida species group = C. albicans, C. parapsilosis, C. tropicalis, C. dubliniensis

TABLE 9-4
T. vaginalis multi-phase amplification
oligos used in multiplex amplification assay

| | | | concentration |
|---|---|---|---|
| Mix | SEQ ID NO: | Oligo type | (pmol/reaction) |
| Target Capture | 3 | Capture | 7.5 |
| Target Capture | 11 | T7 Primer | 1.88 |
| AMP | 15 | NT7 Primer | 8.08 |
| PRO | 11 | T7 primer | 10.75 |
| PRO | 24 | NT7 primer | 12.5 |

TABLE 9-5
Oligonucleotides

| | | Oligo | | |
|---|---|---|---|---|
| Target | Class | SEQ ID NO. | # Bases | Mol. Wt. |
| C. albicans, | TCO | 29 | 50 | 15529 |
| C. tropicalis, | TCO | 30 | 53 | 16513 |
| C. dubliniensis, | NT7 primer | 34 | 21 | 6480 |
| C. parapsilosis | NT7 primer | 35 | 21 | 6447 |
| | T7 primer | 32 | 46 | 14140 |
| | Torch (FAM-Dabcyl) | 38 | 28 | 10633 |
| C. glabrata, | TCO | 31 | 59 | 18365 |
| Control | NT7 primer | 36 | 18 | 5537 |
| | T7 primer | 33 | 49 | 15073 |
| C. glabrata | Torch (HEX-Dabcyl) | 37 | 22 | 8723 |
| Control Torch | Torch (Cy5.5-BBQ) | 63 | 23 | 9279 |
| T. vaginalis | TCO | 3 | 57 | 17609 |
| | NT7 primer | 15 | 22 | 6744 |
| | T7 primer | 11 | 49 | 15050 |
| | Torch (ROX-Acridine) | 24 | 27 | 10694 |

^{a}Lower case = methoxy RNA; Upper case = DNA FAM = Fluorescein; HEX = Hexochloro-Fluorescein; ROX = Carboxy-X-Rhodamine; Cy5.5 = Cyanine 5.5; BBQ = BlackBerry Quencher 650 C9 = 9 carbon chain linker

TABLE 9-6
Torch Oligos

| | Torch | |
|---|---|---|
| Organism | SEQ ID NO | methoxy RNA sequence (5′ → 3′) |
| Candida sp. | 38 | (FAM)ggaauggcgccguggaugguug(C9)cauucc(Dabcyl) |
| C. glabrata | 37 | (HEX)ggaugugacugucaugc(C9)caucc(Dabcyl) |
| Control Torch | 63 | (Cy5.5)gcaug(C9)gugcgaauugggacaugc(BBQ) |
| T. vaginalis | 24 | (Acridine)cgaaguccuucgguuaaaguuc(C9)cuucg(ROX) |

TABLE 9-7
Positivity Summary for Candida species

| | Target | | | | | | |
|---|---|---|---|---|---|---|---|
| | Concentration | | N | % | Average | Average | Average |
| Species | (CFU/mL) | N | Positive | Positive | T-time | RFU Range | T-slope |
| C. albicans | 0 | 15 | 0 | 0.0 | N/A | N/A | N/A |
| | 30 | 15 | 1 | 6.7 | 17.15 | 300.52 | 0.08 |
| | 100 | 15 | 6 | 40.0 | 18.08 | 2280.40 | 0.09 |
| | 150 | 15 | 7 | 46.7 | 17.41 | 2766.52 | 0.08 |
| | 300 | 15 | 12 | 80.0 | 16.92 | 4995.23 | 0.12 |
| | 1000 | 15 | 15 | 100.0 | 15.56 | 6163.80 | 0.16 |
| C. parapsilosis | 0 | 15 | 0 | 0.0 | N/A | N/A | N/A |
| | 3 | 15 | 0 | 0.0 | N/A | −129.07 | N/A |
| | 10 | 15 | 4 | 26.7 | 18.41 | 1478.23 | 0.06 |
| | 30 | 15 | 7 | 46.7 | 19.91 | 2208.79 | 0.05 |
| | 100 | 15 | 10 | 66.7 | 17.18 | 4562.83 | 0.06 |
| | 300 | 15 | 15 | 100.0 | 15.85 | 7181.49 | 0.08 |
| C. tropicalis | 0 | 15 | 0 | 0.0 | N/A | N/A | N/A |
| | 3 | 15 | 1 | 6.7 | 18.33 | 331.88 | 0.04 |
| | 10 | 15 | 4 | 26.7 | 18.36 | 1666.99 | 0.07 |
| | 30 | 15 | 8 | 53.3 | 17.49 | 3447.28 | 0.05 |
| | 100 | 15 | 15 | 100.0 | 17.18 | 4562.83 | 0.06 |
| | 300 | 15 | 15 | 100.0 | 15.85 | 7181.49 | 0.08 |
| | 1000 | 15 | 15 | 100.0 | 14.97 | 6921.05 | 0.09 |
| C. dubliniensis | 0 | 15 | 0 | 0.0 | N/A | N/A | N/A |
| | 10 | 15 | 1 | 6.7 | 18.34 | 291.80 | 0.05 |
| | 30 | 15 | 2 | 13.3 | 20.46 | 609.09 | 0.05 |
| | 100 | 15 | 4 | 26.7 | 18.32 | 2060.29 | 0.05 |
| | 300 | 15 | 12 | 80.0 | 17.47 | 4992.67 | 0.07 |
| | 1000 | 15 | 15 | 100.0 | 15.96 | 6271.16 | 0.11 |
| C. glabrata | 0 | 15 | 0 | 0.0 | N/A | N/A | N/A |
| | 10 | 15 | 0 | 0.0 | N/A | 255.33 | 0.03 |
| | 15 | 15 | 0 | 0.0 | N/A | 258.67 | 0.03 |
| | 30 | 15 | 2 | 13.3 | 24.57 | 524.41 | 0.03 |
| | 50 | 15 | 9 | 60.0 | 24.63 | 1009.85 | 0.03 |
| | 100 | 15 | 15 | 100.0 | 23.82 | 1816.60 | 0.03 |

TABLE 9-8
Positivity summary for T. vaginalis Lysate

| Concen- | | | | | Average | |
|---|---|---|---|---|---|---|
| tration | | N | % | Average | RFU | Average |
| (Cells/mL) | N | Positive | Positive | T-time | Range | T-slope |
| 0 | 15 | 0 | 0.0 | N/A | N/A | N/A |
| 0.0001 | 15 | 1 | 6.7 | 33.95 | −1077.61 | 0.03 |
| 0.0003 | 15 | 3 | 20.0 | 33.36 | −730.76 | 0.03 |
| 0.001 | 15 | 15 | 100.0 | 31.97 | 2380.89 | 0.04 |
| 0.003 | 15 | 15 | 100.0 | 29.38 | 3819.72 | 0.04 |
| 0.01 | 15 | 15 | 100.0 | 25.74 | 5202.32 | 0.04 |

Using a normal Probit model, there is a 50% probability (95% confidence level) of detecting T. vaginalis present at 0.0004 (0.0003-0.0005) cells/mL, and a 95% probability (95% confidence level) of detecting T. vaginalis present at 0.001 (0.007-0.0003) cells/mL. Using a Gompertz Probit model, there is a 50% probability (95% confidence level) of detecting T. vaginalis present at 0.00004 (0.0003-0.0006) cells/mL cells/mL, and a 95% probability (95% confidence level) of detecting T. vaginalis present at 0.0008 (0.0006-0.0016) cells/mL) cells/mL. Probit values species are shown in Tables 9-9 and 9-10.

TABLE 9-9
Probit Summary, Normal Model.

| Target | 50% Probability (95% CL) | 95% Probability (95% CL) |
|---|---|---|
| C. albicans | 136 (94-193) CFU/mL | 627 (374-1927) CFU/mL |
| C. parapsilosis | 34 (21-56) CFU/mL | 291 (149-992) CFU/mL |
| C. tropicalis | 19 (12-30) CFU/mL | 106 (59-332) CFU/mL |
| c. dubliniensis | 122 (75-199) CFU/mL | 911 (462-3389) CFU/mL |
| C. glabrata | 45 (37-55) CFU/mL | 77 (61-146) CFU/mL |
| T. vaginalis | 0.0004 (0.0003-0.0005) cells/mL | 0.001 (0.007-0.003) cells/mL |

TABLE 9-10
Probit Summary, Gompertz Model.

| Target | 50% Probability (95% CL) | 95% Probability (95% CL) |
|---|---|---|
| C. albicans | 151 (99-211) CFU/mL | 482 (313-1423) CFU/mL |
| C. parapsilosis | 42 (24-67) CFU/mL | 225 (132-584) CFU/mL |
| C. tropicalis | 23 (14-34) CFU/mL | 77 (49-183) CFU/mL |
| C. dubliniensis | 148 ( 90-226) CFU/mL | 565 (347-1439) CFU/mL |
| C. glabrata | 46 (39-58) CFU/mL | 67 (55-145) |
| T. vaginalis | 0.00004 (0.0003-0.0006) cells/mL | 0.0008 (0.0006-0.0016) cells/mL |

### Example 10

### In Silico Specificity Analysis

In silico analysis of T. vaginalis, Candida, and control oligonucleotides (Table 9.5) and control oligonucleotides (Table 9.5) was conducted to assess the likelihood that the system would cross-react with undesired targets or form undesirable inter or intra-molecular interactions. Oligonucleotides were also subjected to interaction analysis using the OLIGO 7 and OligoAnalyzer applications. Potential interactions with a forward and reverse primer pair with subject start positions equal to or less than 300 bp with or without an internal Torch sequence were queried. Matches were filtered for, forward primers in the same direction as subject sequence, reverse primers in reverse direction as subject sequence, and Torch sequence in same direction as subject sequence. BLAST results using the T. vaginalis and control oligonucleotides as queries against human and GenBank databases were examined for subjects that appeared to have the potential to be amplified and detected in the ACV/TV system. Among all datasets queried (bacterial, fungal, viral, human) by BLAST, one primer-only interaction of possible interest was identified: HIV-1 (accession no. AF254708) with oligos SEQ ID NO: 36 and 11. HIV-1 was tested in panel 11 in cross reactivity testing (see below) and showed no sign of either cross reactivity or interference. Amplification of HIV, with these two oligoes is therefore negligible.

### Example 11

### Cross-Reactivity Testing

With the addition of T. vaginalis as a target in the ROX channel, cross reactivity was evaluated in four-plex assay panels against a variety of organisms. Multiphase amplification was performed as described above using the T. vaginalis oligos as described. Panels and results are shown in Table 10-1. 5 replicates of each panel were tested to determine if any cross reactivity occurred. (Note: Panels 10 and 12 are not listed because they contained target species.)

TABLE 11-1
Summary of average RFU range of cross reactivity panels

| | | | | Average | Average | Average | Average |
|---|---|---|---|---|---|---|---|
| | | | | RFU | RFU | RFU | RFU |
| | | | | range | range | range | control |
| | | Final | | C. spp | C. gla | TV | Torch |
| Panel | Organism | Conc. | Units | (FAM) | (HEX) | (ROX) | (Cy5.5) |
| 1 | Acinetobacter iwoffii | 1.00 × 10⁶ | CFU/ml | −118.00 | −62.27 | −1036.93 | 5549.80 |
| | Actinomyces israelii | 5.00 × 10⁹ | copies rRNA/ml | | | | |
| | Alcaligenes faecalis | 1.00 × 10⁶ | CFU/ml | | | | |
| | Atopobium vaginae | 5.00 × 10⁹ | copies rRNA/ml | | | | |
| 2 | Bacteroides fragilis | 1.00 × 10⁶ | CFU/ml | −111.00 | −27.13 | −1049.47 | 5519.60 |
| | Bifidobacterium adolescentis | 1.00 × 10⁶ | CFU/ml | | | | |
| | Campylobacter jejuni | 1.00 × 10⁶ | CFU/ml | | | | |
| | Chlamydia trachomatis | 1.00 × 10⁵ | IFU/ml | | | | |
| 3 | Candida krusei | 1.00 × 10⁶ | CFU/ml | −104.93 | −19.53 | −983.53 | 5484.27 |
| | Candida lusitaniae | 1.00 × 10⁶ | CFU/ml | | | | |
| | Clostridium difficile | 1.00 × 10⁶ | CFU/ml | | | | |
| | Corynebacterium genitalium | 1.00 × 10⁶ | CFU/ml | | | | |
| 4 | Cryptococcus neoformans | 1.00 × 10⁶ | CFU/ml | −118.73 | −61.67 | −1073.80 | 5778.47 |
| | Eggerthella lenta | 1.00 × 10⁶ | CFU/ml | | | | |
| | Enterobacter cloacae | 1.00 × 10⁶ | CFU/ml | | | | |
| | Enterococcus faecalis | 1.00 × 10⁶ | CFU/ml | | | | |
| 5 | Escherichia coli | 1.00 × 10⁶ | CFU/ml | −110.13 | −24.53 | −973.40 | 5552.73 |
| | Haemophilus ducreyi | 1.00 × 10⁶ | CFU/ml | | | | |
| | Klebsiella pneumoniae | 1.00 × 10⁶ | CFU/ml | | | | |
| | Listeria monocytogenes | 1.00 × 10⁶ | CFU/ml | | | | |
| 6 | Lactobacillus acidophilus | 1.00 × 10⁶ | CFU/ml | −116.00 | −62.80 | −1065.07 | 5505.33 |
| | Lactobacillus iners | 1.00 × 10⁶ | CFU/ml | | | | |
| | Lactobacillus mucosae | 1.00 × 10⁶ | CFU/ml | | | | |
| | Leptotrichia bucalis | 1.00 × 10⁶ | CFU/ml | | | | |
| 7 | Mobiluncus curtisii | 5.00 × 10⁹ | copies rRNA/ml | 1858.20 | 91.60 | −1008.27 | 5565.33 |
| | Mycoplasma genitalium | 1.00 × 10⁶ | CFU/ml | | | | |
| | Mycoplasma hominis | 5.00 × 10⁹ | copies rRNA/ml | | | | |
| | Neisseria gonorrhoeae | 1.00 × 10⁶ | CFU/ml | | | | |
| 8 | Peptostreptococcus magnus | 1.00 × 10⁶ | CFU/ml | −106.13 | 24.73 | −993.53 | 5458.20 |
| | Prevotella bivia | 1.00 × 10⁶ | CFU/ml | | | | |
| | Propionibacterium acnes | 1.00 × 10⁶ | CFU/ml | | | | |
| | Proteus vulgaris | 1.00 × 10⁶ | CFU/ml | | | | |
| 9 | Staphylococcus aureus | 1.00 × 10⁶ | CFU/ml | −111.00 | 19.47 | −997.07 | 5531.07 |
| | Staphylococcus epidermidis | 1.00 × 10⁶ | CFU/ml | | | | |
| | Streptococcus agalactiae | 1.00 × 10⁶ | CFU/ml | | | | |
| | Streptococcus pyogenes | 1.00 × 10⁶ | CFU/ml | | | | |
| 11 | Herpes simplex virus I | 1.00 × 10⁵ | TCID 50/ml | −113.20 | −21.73 | −987.00 | 5370.80 |
| | Herpes simplex virus II | 1.00 × 10⁵ | TCID 50/ml | | | | |
| | HIV | 1.00 × 10⁶ | copies/ml | | | | |
| 13 | Gardnerella vaginalis | 1.00 × 10⁶ | CFU/ml | −114.53 | −67.33 | −1051.20 | 5539.27 |
| | Lactobacillus crispatus | 1.00 × 10⁶ | CFU/ml | | | | |
| | Lactobacillus gasseri | 1.00 × 10⁶ | CFU/ml | | | | |
| | Lactobacillus jensenii | 1.00 × 10⁶ | CFU/ml | | | | |

One replicate of Panel 7 was positive in the FAM channel. All other replicates were negative in all channels. Cross reactivity against the organisms in panel 7 were re-valuated. Upon retesting these organisms, no cross reaction was observed and all replicates were negative. It was concluded that the false positive replicate found in Panel 7 was due to a random contamination event.

### Example 12

### Interference in the ROX Channel for T. vaginalis Detection

Five replicates of cross reactivity panels were tested in the presence of Trichomonas vaginalis at 3× limit of detection (0.003 cells/mL). Multiphase amplification was performed as described. There was no interference observed in the presence of any panels in the ROX channel, and all replicates were positive as expected. The control Torch (Cy5.5, RTF2) were valid for all replicates. The results demonstrated that the T. vaginalis oligos were able to detect T. vaginalis in the presence of the various organisms in panels 1-9, 11, and 13 from the example above.

TABLE 12-1
Interference Panel Summary. Average RFU
ranges of Trichomonas vaginalis in the ROX
channel and control Torch in the Cy5.5 channel.
Each panel contained 0.003 cells/mL T. vaginalis.

| | Average RFU | Average |
|---|---|---|
| | Range TV | RFU Range |
| Panel | (ROX) | IC (Cy5.5) |
| 1 | 3533.53 | 5325.40 |
| 2 | 3213.67 | 5339.80 |
| 3 | 3659.33 | 5630.93 |
| 4 | 4636.80 | 5556.67 |
| 5 | 4317.27 | 5594.47 |
| 6 | 4257.67 | 5388.80 |
| 7 | 4124.00 | 5463.93 |
| 8 | 3943.73 | 5441.47 |
| 9 | 4701.40 | 5618.93 |
| 11 | 3328.27 | 5501.60 |
| 13 | 4049.60 | 5716.07 |

Five replicates of cross reactivity panels were tested in the presence of Trichomonas vaginalis at 3× limit of detection (0.003 cells/mL). Multiphase amplification was performed as described. There was no interference observed in the presence of any panels in the ROX channel, and all replicates were positive as expected. The control Torch (Cy5.5, RTF2) were valid for all replicates. The results demonstrated that the T. vaginalis oligos were able to detect T. vaginalis in the presence of the various organisms in panels 1-9, 11, and 13 from the example above.

### Example 13

### T. vaginalis Clinical Sample Testing

Seventeen (17) vaginal swab clinical specimens initially testing positive by Aptima Trichomonas Assay were tested neat with the Aptima CV/TV multiplex assay. Multiphase amplification was performed as described using the T. vaginalis oligos TCO SEQ ID NO. 3, NT7 primer SEQ ID NO. 15, T7 primer SEQ ID NO. 11, and Torch SEQ ID NO. 24. One rep of each neat sample was taken for testing. 15/17 (88%) of samples yielded valid results with the CV/TV multiplex assay and were all positive for T. vaginalis. 3/15 (20%) of valid samples were positive for both Candida species and T. vaginalis. The invalid samples were determined to be invalid due to absence of signal in all channels and had a recorded instrument error, with the likely cause being insufficient sample volume.

TABLE 13-1
ACV/TV Multiplex Neat Testing of samples having >1000 RLUs
in ATV IVD assay and considered positive for T. vaginalis, n = 1.

| | ACV/TV Muliplex T-time | | | | ACV/TV |
|---|---|---|---|---|---|
| Sample | C. spp | C. gla | TV | IC | Interpretation |
| 10010 | 9.05 | — | 7.00 | — | TRICH POS |
| 10052 | — | — | 7.79 | 29.62 | TRICH POS |
| 11207 | — | — | — | — | Invalid |
| 12045 | | — | 8.37 | 26.49 | TRICH POS |
| 12049 | 8.82 | — | 7.35 | | TRICH POS |
| 13023 | — | — | — | — | Invalid |
| 13186 | — | — | 19.23 | 17.06 | TRICH POS |
| 17014 | — | — | 12.96 | 19.53 | TRICH POS |
| 11230 | — | — | 5.06 | — | TRICH POS |
| 11241 | — | — | 5.26 | — | TRICH POS |
| 11245 | 7.63 | — | 10.01 | — | TRICH POS |
| 12011 | — | — | 5.35 | — | TRICH POS |
| 12030 | — | — | 5.47 | — | TRICH POS |
| 14227 | — | — | 30.76 | 16.61 | TRICH POS |
| 14274 | — | — | 5.11 | — | TRICH POS |
| 17032 | — | — | 12.26 | 18.14 | TRICH POS |
| 17040 | — | — | 10.25 | 20.09 | TRICH POS |
| STM_Negative | — | — | — | 16.23 | — |

Serial dilutions with STM were then created following initial testing and tested comparatively against Aptima CV/TV multiplex and Aptima Trichomonas Vaginalis IVD assays. Dilutions in STM ranging from 1:5 and 1:10,000 were done for clinical samples depending on T-time of neat sample testing. Dilution of 1:10 was done for samples 11207 and 13023 that were determined invalid from neat sample testing. Each dilution was run with the CV/TV multiplex assay and retested with Aptima Trichomonas Vaginalis assay. Previous invalid samples were valid upon retesting with 1:10 dilution. All samples, including previous invalid samples, agreed with Aptima Trichomonas Vaginalis assay interpretation.

TABLE 13-2
Clinical Sample Dilution Comparison.

| | | ACV/TV Multiplex T-time | | | | ACV/TV | ATV IVD | ATV IVD |
|---|---|---|---|---|---|---|---|---|
| Sample | Dilution | C.spp | C.gla | TV | IC | Interpretation | RLU (/1000) | Interpretation |
| 11230 | 1:1000 | — | — | 11.28 | 19.62 | TRICH POS | 1535 | TRICH POS |
| | 1:10,000 | — | — | 12.84 | 17.12 | TRICH POS | 1567 | TRICH POS |
| 11241 | 1:1000 | — | — | 10.77 | 18.76 | TRICH POS | 1613 | TRICH POS |
| | 1:10,000 | — | — | 12.87 | 16.88 | TRICH POS | 1536 | TRICH POS |
| 11245 | 1:1000 | 12.02 | — | 18.87 | 17.61 | TRICH POS | 1577 | TRICH POS |
| | 1:10,000 | 13.64 | — | 23.44 | 16.76 | TRICH POS | 1476 | TRICH POS |
| 12011 | 1:1000 | — | — | 10.71 | 18.95 | TRICH POS | 1531 | TRICH POS |
| | 1:10,000 | — | — | 12.92 | 16.91 | TRICH POS | 1580 | TRICH POS |
| 12030 | 1:1000 | — | — | 11.10 | 18.86 | TRICH POS | 1552 | TRICH POS |
| | 1:10,000 | — | — | 13.49 | 17.07 | TRICH POS | 1614 | TRICH POS |
| 14227 | 1:10 | — | — | — | 16.10 | TRICH POS | 29 | TRICH POS |
| | 1:5 | — | — | — | 16.47 | TRICH POS | 17 | TRICH POS |
| 14274 | 1:1000 | — | — | 10.80 | 19.79 | TRICH POS | 1550 | TRICH POS |
| | 1:10,000 | — | — | 12.97 | 17.53 | TRICH POS | 1558 | TRICH POS |
| 17032 | 1:1000 | — | — | 21.37 | 16.09 | TRICH POS | 1370 | TRICH POS |
| | 1:10,000 | — | — | 28.78 | 16.01 | TRICH POS | 409 | TRICH POS |
| 17040 | 1:1000 | — | — | 17.80 | 16.29 | TRICH POS | 1514 | TRICH POS |
| | 1:10,000 | — | — | 21.95 | 16.13 | TRICH POS | 1363 | TRICH POS |
| 10010 | 1:100 | 11.27 | — | 10.21 | 20.67 | TRICH POS | 1547 | TRICH POS |
| | 1:1000 | 12.67 | — | 12.22 | 22.43 | TRICH POS | 1520 | TRICH POS |
| 10052 | 1:100 | — | — | 11.39 | 20.07 | TRICH POS | 1483 | TRICH POS |
| | 1:1000 | — | — | 13.30 | 17.71 | TRICH POS | 1486 | TRICH POS |
| 12045 | 1:100 | — | — | 12.42 | 18.94 | TRICH POS | 1488 | TRICH POS |
| | 1:1000 | — | — | 15.06 | 17.64 | TRICH POS | 1484 | TRICH POS |
| 12049 | 1:100 | 11.07 | — | 10.17 | 20.83 | TRICH POS | 1526 | TRICH POS |
| | 1:1000 | 12.47 | — | 12.06 | 21.89 | TRICH POS | 1533 | TRICH POS |
| 13186 | 1:10 | 16.45 | — | 22.08 | 16.85 | TRICH POS | 1225 | TRICH POS |
| | 1:100 | — | — | 28.34 | 17.02 | TRICH POS | 255 | TRICH POS |
| 17014 | 1:100 | — | — | 18.17 | 16.83 | TRICH POS | 1422 | TRICH POS |
| | 1:1000 | — | — | 22.69 | 16.56 | TRICH POS | 1222 | TRICH POS |
| 11207 | 1:10 | 11.90 | — | 25.84 | 19.09 | TRICH POS | 1327 | TRICH POS |
| 13023 | 1:10 | 10.39 | — | 17.55 | 19.90 | TRICH POS | 1482 | TRICH POS |

### EMBODIMENTS

Embodiment 1. An amplification oligonucleotide for use in amplifying a T. vaginalis target nucleic acid sequence in a sample comprising: a promoter primer containing 15-30 contiguous bases having at least 90% complementarity to a region of SEQ ID NO: 176 or a complement thereof.

Embodiment 2. The amplification oligonucleotide of Embodiment 1, wherein the promoter primer comprises a 5′ promoter sequence for a T7 RNA polymerase.

Embodiment 3. The amplification oligonucleotide of Embodiment 2, wherein the promoter sequence for the T7 RNA polymerase comprises SEQ ID NO: 65 or 66.

Embodiment 4. The amplification oligonucleotide of Embodiment 2, wherein the promoter primer comprises a nucleic acid sequence having at least 90% identity to SEQ ID NO: 42, 43, 44, 45, 46, 47, or 48.

Embodiment 5. The amplification oligonucleotide of Embodiment 4, wherein the promoter primer comprises a nucleic acid sequence having at least 90% identity to SEQ ID NO: 4, 5, 6, 7, 8, 9, 10, 11, or 12.

Embodiment 6. A set of amplification oligonucleotides comprising the amplification oligonucleotide of any one of Embodiments 1-5 and one or more additional amplification oligonucleotides suitable for use in amplification of one or more additional target nucleic acids.

Embodiment 7. An amplification oligonucleotide or use in amplifying a T. vaginalis target nucleic acid sequence in a sample comprising: a non-promoter primer containing 15-30 contiguous bases having at least 90% complementarity to a region of SEQ ID NO: 177 or a complement thereof.

Embodiment 8. The amplification oligonucleotide of Embodiment 6, wherein the non-promoter primer comprises a nucleic acid sequence having at least 90% identity to SEQ ID NO: 49, 50, 51, 52, 53, 54, or 55.

Embodiment 9. The amplification oligonucleotide of Embodiment 7, wherein the non-promoter primer comprises a nucleic acid sequence having at least 90% identity to SEQ ID NO: 13, 14, 15, 16, 17, 18, or 19.

Embodiment 10. A set of amplification oligonucleotides comprising the non-promoter primer of any one of Embodiments 7-9 and one or more additional non-promoter primers suitable for use in amplification of one or more additional target nucleic acids.

Embodiment 11. A detection oligonucleotide for detecting a T. vaginalis target nucleic acid amplification product comprising: a nucleic acid sequence having at least 90% identity to SEQ ID NO: 56, 57, 58, 59, 60, 61, or 62.

Embodiment 12. The detection oligonucleotide of Embodiment 11, wherein the detection oligonucleotide is a conformation-sensitive hybridization probe that produces a detectable signal when hybridized to an amplification product of a T. vaginalis target nucleic acid.

Embodiment 13. The detection oligonucleotide of Embodiment 12, wherein the detection oligonucleotide contains a fluorophore and optionally a quencher.

Embodiment 14. The detection oligonucleotide of Embodiment 13, wherein the detection oligonucleotide is a molecular torch.

Embodiment 15. The detection oligonucleotide of Embodiment 11, wherein the detection oligonucleotide contains a nucleic acid sequence having at least 90% identity to SEQ ID NO: 20, 21, 22, 23, 24, 25, 26, 27, or 28.

Embodiment 16. A set of detection oligonucleotides comprising the detection oligonucleotide of any one of Embodiments 11-15 and one or more additional detection oligonucleotides suitable for use in detecting the amplification products of one or more additional target nucleic acids.

Embodiment 17. A target capture oligonucleotide (TCO) for use in capturing T vaginalis target nucleic acid in a sample wherein the TCO comprises a nucleic acid sequence having at least 90% identity to SEQ ID NO: 39, 40, or 41 and an immobilized capture probe-binding region that binds to an immobilized capture probe.

Embodiment 18. The TCO of Embodiment 17, wherein the immobilized capture probe-binding region comprises a nucleic acid sequence capable of stably hybridizing under assay conditions to an oligonucleotide that is bound to the capture probe.

Embodiment 19. The TCO of Embodiment 18, wherein the TCO comprises a nucleic acid sequence having at least 90% identity to SEQ ID NO: 1, 2, or 3.

Embodiment 20. A set of TCOs comprising, the TCO of any one of Embodiments 17-19 and one or more additional TCOs for use in capturing one or more additional target nucleic acids.

Embodiment 21. A composition for detecting T. vaginalis in a sample comprising:

(a) a promoter primer comprising the amplification oligonucleotide of any one of Embodiments 1-5;

(b) a non-promoter primer comprising the amplification oligonucleotide of any one of Embodiments 7-9;

(c) a detection oligonucleotide comprising the detection oligonucleotide of any of Embodiments 11-15; and

(d) optionally a target capture oligonucleotide (TCO) comprising the TCO of any one of Embodiments 17-19.

Embodiment 22. The composition of Embodiment 21, wherein the promoter primer is present in a target capture mixture, the non-promoter primer is present in a first phase amplification mixture, and the promoter primer and detection oligonucleotide are present in a second phase amplification mixture.

Embodiment 23. The composition of Embodiment 22, wherein the target capture mixture further comprises the TCO.

Embodiment 24. The composition of Embodiment 22, wherein the first phase amplification mixture contains one or more of: reverse transcriptase, RNA polymerase, deoxyribonucleotide triphosphates and ribonucleotides triphosphates.

Embodiment 25. The composition of any one of Embodiments 21-24, further comprising an immobilized capture probe, wherein the immobilized capture probe contains a first binding pair member the binds to a second binding pair member present on the TCO.

Embodiment 26. The composition of Embodiment 25, wherein the immobilized capture probe comprises magnetically attractable particles.

Embodiment 27. The composition of Embodiment 22, wherein the first phase amplification reaction mixture lacks the promoter primer.

Embodiment 28. The composition of Embodiment 21, wherein the target capture mixture contains one or more additional promoter primers, the first phase amplification mixture contains one or more additional non-promoter primers, and the second amplification mixture contains one or more additional more promoter primers and one or more detection oligonucleotides, wherein the one or more additional promoter primers, non-promoter primers, and detection oligonucleotides and suitable for amplification and detection of species other than T. vaginalis.

Embodiment 29. The method of Embodiment 24, wherein at least one of the species other than T. vaginalis is a Candida species.

Embodiment 30. The composition of Embodiment 21, wherein the TCO comprises the nucleotide sequence of SEQ ID NO: 3, the T7 primer comprises the nucleotide sequence of SEQ ID NO: 11, the NT7 primer comprises the nucleotide sequence of SEQ ID NO: 15, and the Torch comprises the nucleotide sequence of SEQ ID NO: 24.

Embodiment 31. The composition of Embodiment 21, wherein the TCO comprises the nucleotide sequence of SEQ ID NO: 3, the T7 primer comprises the nucleotide sequence of SEQ ID NO: 4, the NT7 primer comprises the nucleotide sequence of SEQ ID NO: 14, and the Torch comprises the nucleotide sequence of SEQ ID NO: 20.

Embodiment 32. The composition of Embodiment 21, wherein the TCO comprises the nucleotide sequence of SEQ ID NO: 3, the T7 primer comprises the nucleotide sequence of SEQ ID NO: 4, the NT7 primer comprises the nucleotide sequence of SEQ ID NO: 14, and the Torch comprises the nucleotide sequence of SEQ ID NO: 21.

Embodiment 33. The composition of Embodiment 21, wherein the TCO comprises the nucleotide sequence of SEQ ID NO: 3, the T7 primer comprises the nucleotide sequence of SEQ ID NO: 4, the NT7 primer comprises the nucleotide sequence of SEQ ID NO: 13, and the Torch comprises the nucleotide sequence of SEQ ID NO: 21.

Embodiment 34. The composition of Embodiment 21, wherein the TCO comprises the nucleotide sequence of SEQ ID NO: 3, the T7 primer comprises the nucleotide sequence of SEQ ID NO: 9, the NT7 primer comprises the nucleotide sequence of SEQ ID NO: 15, and the Torch comprises the nucleotide sequence of SEQ ID NO: 23.

Embodiment 35. The composition of Embodiment 21, wherein the TCO comprises the nucleotide sequence of SEQ ID NO: 2, the T7 primer comprises the nucleotide sequence of SEQ ID NO: 4, the NT7 primer comprises the nucleotide sequence of SEQ ID NO: 14, and the Torch comprises the nucleotide sequence of SEQ ID NO: 20.

Embodiment 36. The composition of Embodiment 21, wherein the TCO comprises the nucleotide sequence of SEQ ID NO: 2, the T7 primer comprises the nucleotide sequence of SEQ ID NO: 4, the NT7 primer comprises the nucleotide sequence of SEQ ID NO: 14, and the Torch comprises the nucleotide sequence of SEQ ID NO: 21.

Embodiment 37. The composition of Embodiment 21, wherein the TCO comprises the nucleotide sequence of SEQ ID NO: 2, the T7 primer comprises the nucleotide sequence of SEQ ID NO: 4, the NT7 primer comprises the nucleotide sequence of SEQ ID NO: 13, and the Torch comprises the nucleotide sequence of SEQ ID NO: 21.

Embodiment 38. The composition of Embodiment 21, wherein the TCO comprises the nucleotide sequence of SEQ ID NO: 2, the T7 primer comprises the nucleotide sequence of SEQ ID NO: 9, the NT7 primer comprises the nucleotide sequence of SEQ ID NO: 15, and the Torch comprises the nucleotide sequence of SEQ ID NO: 23.

Embodiment 39. The composition of Embodiment 21, wherein the TCO comprises the nucleotide sequence of SEQ ID NO: 2, the T7 primer comprises the nucleotide sequence of SEQ ID NO: 4, the NT7 primer comprises the nucleotide sequence of SEQ ID NO: 13, and the Torch comprises the nucleotide sequence of SEQ ID NO: 20.

Embodiment 40. A method of detecting T. vaginalis in a sample comprising:
(a) contacting the sample with a promoter primer, under conditions allowing hybridization of the promoter primer to a first portion of a T. vaginalis target nucleic acid sequence, thereby generating a pre-amplification hybrid that comprises the promoter primer and the target nucleic acid sequence
   wherein the promoter primer comprises a nucleic acid sequence having at least 90% complementarity to a region of SEQ ID NO: 176 or a complement thereof.
(b) isolating the pre-amplification hybrid by target capture onto a solid support followed by washing to remove any of the promoter primer that did not hybridize to the first portion of the target nucleic acid sequence in step (a);
(c) amplifying, in a first phase amplification reaction mixture, at least a portion of the target nucleic acid sequence of the pre-amplification hybrid isolated in step (b) in a first phase, substantially isothermal, transcription-associated amplification reaction under conditions that support linear amplification thereof, but do not support exponential amplification thereof, thereby resulting in a reaction mixture comprising a first amplification product,
   wherein the first phase amplification reaction mixture comprises a non-promoter primer, the non-promoter being complementary to a portion of an extension product of the promoter primer, and comprising a nucleic acid sequence having at least 90% complementarity to a region of SEQ ID NO: 177 or a complement thereof
   wherein the first amplification product is not a template for nucleic acid synthesis during the first phase, substantially isothermal, transcription-associated amplification reaction;
(d) combining the reaction mixture comprising the first amplification product with additional promoter primer, to produce a second phase amplification reaction mixture,
   wherein the second phase amplification reaction mixture additionally comprises a detection oligonucleotide;
(e) performing, in a second phase, a substantially isothermal, transcription-associated amplification reaction in the second phase amplification reaction mixture, an exponential amplification of the first amplification product, thereby synthesizing a second amplification product;
(f) detecting, with the detection oligonucleotide at regular time intervals, synthesis of the second amplification product in the second phase amplification reaction mixture; and
(g) quantifying the target nucleic acid sequence in the sample using results from step (f).

Embodiment 41. The method of Embodiment 40, wherein the promoter primer comprises a 5′ promoter sequence for a T7 RNA polymerase.

Embodiment 42. The method of Embodiment 41, wherein the promoter sequence for the T7 RNA polymerase comprises SEQ ID NO: 65 or 66.

Embodiment 43. The method of Embodiment 41, wherein the promoter primer comprises a nucleic acid sequence having at least 90% identity to SEQ ID NO: 42, 43, 44, 45, 46, 47, or 48.

Embodiment 44. The method of Embodiment 43, wherein the promoter primer comprises a nucleic acid sequence having at least 90% identity to SEQ ID NO: 4, 5, 6, 7, 8, 9, 10, 11, or 12.

Embodiment 45. The method of Embodiment 40, wherein the non-promoter primer is enzymatically extended in the first phase isothermal transcription-associated amplification reaction.

Embodiment 46. The method of Embodiment 45, wherein the non-promoter primer comprises a nucleic acid sequence having at least 90% identity to SEQ ID NO: 49, 50, 51, 52, 53, 54, or 55.

Embodiment 47. The method of Embodiment 46, wherein the non-promoter primer comprises a nucleic acid sequence having at least 90% identity to SEQ ID NO: 13, 14, 15, 16, 17, 18, or 19.

Embodiment 48. The method of Embodiment 40, wherein isolating the pre-amplification hybrid comprises contacting the sample with a target capture oligonucleotide (TCO), wherein the pre-amplification hybrid comprises the target nucleic acid sequence hybridized to each of the TCO and promoter primer.

Embodiment 49. The method of Embodiment 48, wherein the TCO comprises a nucleic acid sequence having at least 90% identity to SEQ ID NO: 39, 40, or 41.

Embodiment 50. The method of Embodiment 48, wherein the TCO comprises a nucleic acid sequence having at least 90% identity to SEQ ID NO: 1, 2, or 3.

Embodiment 51. The method of Embodiment 40, wherein the solid support comprises an immobilized capture probe.

Embodiment 52. The method of Embodiment 51, wherein the immobilized capture probe comprises magnetically attractable particles.

Embodiment 53. The method of Embodiment 40, wherein each of the first and second phase isothermal transcription-associated amplification reactions comprise an RNA polymerase and a reverse transcriptase, and wherein the reverse transcriptase comprises an endogenous RNase H activity.

Embodiment 54. The method of Embodiment 40, wherein the first phase amplification reaction mixture lacks free promoter primer.

Embodiment 55. The method of Embodiment 40, wherein the first amplification product of step (c) is a cDNA molecule with the same polarity as the target nucleic acid sequence in the sample, and wherein the second amplification product of step (e) is an RNA molecule.

Embodiment 56. The method of Embodiment 40, wherein the detection oligonucleotide in step (d) is a conformation-sensitive hybridization probe that produces a detectable signal when hybridized to the second amplification product.

Embodiment 57. The method of Embodiment 56, wherein the detection oligonucleotide in step (d) is a fluorescently labeled sequence-specific hybridization probe.

Embodiment 58. The method of Embodiment 57, wherein the detection oligonucleotide contains a region of at least 90% complementarity to a region of SEQ ID NO: 178 or a complement thereof.

Embodiment 59. The method of Embodiment 58, wherein the detection oligonucleotide comprises a nucleic acid sequence having at least 90% identity to SEQ ID NO: 56, 57, 58, 59, 60, 61, or 62.

Embodiment 60. The method of Embodiment 59, wherein the detection oligonucleotide comprises a nucleic acid sequence having at least 90% identity to SEQ ID NO: 20, 21, 22, 23, 24, 25, 26, 27, or 28.

Embodiment 61. The method of Embodiment 40, wherein step (g) comprises quantifying the target nucleic acid sequence in the sample using a calibration curve and results from step (f).

Embodiment 62. The method of Embodiment 40, wherein the method comprises two or more different promoter primers and two or more different non-promoter primers, wherein the two or more different promoter primers and the two or more different non-promoter primers amplify different target nucleic acids to produce two or more different amplification products.

Embodiment 63. The method of Embodiment 62, further comprising two or more different amplification products are detected using two or more different detection oligonucleotides.

Embodiment 63. The method of Embodiment 62, wherein the two or more different target nucleic acids are from different species.

Embodiment 64. The method of Embodiment 63, wherein the different species are Candida species.

What is claimed is:

1. An amplification oligonucleotide for use in amplifying a T. vaginalis target nucleic acid sequence in a sample comprising: a promoter primer comprising a nucleic acid sequence having target specific sequence with at least 90% identity to SEQ ID NO: 42, 43, 44, 45, 46, 47, 48, or a complement thereof, and having a promoter sequence for a T7 RNA polymerase joined at its 5′ end.

2. The amplification oligonucleotide of claim 1, wherein the promoter sequence for the T7 RNA polymerase comprises SEQ ID NO: 65 or 66.

3. The amplification oligonucleotide of claim 1, wherein the promoter primer comprises a nucleic acid sequence having at least 90% identity to SEQ ID NO: 4, 5, 6, 7, 8, 9, 10, 11, or 12.

4. A set of amplification oligonucleotides comprising the amplification oligonucleotide of any one of claims 1-3 and one or more additional amplification oligonucleotides suitable for use in amplification of one or more additional target nucleic acids.

5. An amplification oligonucleotide or use in amplifying a T. vaginalis target nucleic acid sequence in a sample comprising: a non-promoter primer, wherein the non-promoter primer comprises a nucleic acid sequence having at least 90% identity to SEQ ID NO: 13, 14, 15, 16, 17, 18, 19, or a complement thereof.

6. A set of amplification oligonucleotides comprising the non-promoter primer of claim 5 and one or more additional non-promoter primers suitable for use in amplification of one or more additional target nucleic acids.

7. A detection oligonucleotide for detecting a T. vaginalis target nucleic acid amplification product comprising: a nucleic acid sequence having a target specific sequence that is at least 90% identity to SEQ ID NO: 56, 57, 58, 59, 60, 61, 62, or a complement thereof, and wherein the detection oligonucleotide contains a fluorophore and optionally a quencher.

8. The detection oligonucleotide of claim 7, wherein the detection oligonucleotide contains a nucleic acid sequence having at least 90% identity to SEQ ID NO: 20, 21, 22, 23, 24, 25, 26, 27, or 28.

9. A set of detection oligonucleotides comprising the detection oligonucleotide of claim 7 or 8 and one or more additional detection oligonucleotides suitable for use in detecting the amplification products of one or more additional target nucleic acids.

10. A target capture oligonucleotide (TCO) for use in capturing T. vaginalis target nucleic acid in a sample wherein (i) the TCO comprises a nucleic acid sequence having a target specific sequence that is at least 90% identity to SEQ ID NO: 39, 40, or 41 and an immobilized capture probe-binding region that binds to an immobilized capture probe; or (ii) the TCO comprises a nucleic acid sequence having at least 90% identity to SEQ ID NO: 1, 2, or 3.

11. A composition for detecting T. vaginalis in a sample comprising:
(a) a promoter primer comprising the amplification oligonucleotide of any one of claims 1-3;
(b) a non-promoter primer comprising the amplification oligonucleotide of any one of claim 5 or 6;
(e) a detection oligonucleotide comprising the detection oligonucleotide of any of claims 7-9; and
(f) optionally a target capture oligonucleotide (TCO) comprising the TCO of claim 10.

12. The composition of claim 11, wherein the promoter primer is present in a target capture mixture, the non-promoter primer is present in a first phase amplification mixture, and the promoter primer and detection oligonucleotide are present in a second phase amplification mixture.

13. The composition of claim 12, wherein the target capture mixture contains one or more additional promoter primers, the first phase amplification mixture contains one or more additional non-promoter primers, and the second phase amplification mixture contains one or more additional promoter primers and one or more additional detection oligonucleotides, wherein the one or more additional promoter primers, non-promoter primers, and detection oligonucleotides and suitable for amplification and detection of species other than T. vaginalis.

14. The method of claim 13, wherein at least one of the species other than T. vaginalis is a Candida species.

15. A method of detecting T. vaginalis in a sample comprising:
(a) contacting the sample with a promoter primer, under conditions allowing hybridization of the promoter primer to a first portion of a T. vaginalis target nucleic acid sequence, thereby generating a pre-amplification hybrid that comprises the promoter primer and the target nucleic acid sequence
wherein the promoter primer comprises a nucleic acid sequence having at least 90% identity to SEQ ID NO: 42, 43, 44, 45, 46, 47, 48, or a complement thereof, and having a promoter sequence for a T7 RNA polymerase joined at its 5′ end;
(b) isolating the pre-amplification hybrid by target capture onto a solid support, wherein target capture comprises contacting the sample with a target capture oligonucleotide (TCO), wherein the pre-amplification hybrid comprises the target nucleic acid sequence hybridized to each of the TCO and promoter primer, followed by washing to remove any of the promoter primer that did not hybridize to the first portion of the target nucleic acid sequence in step (a);
(c) amplifying, in a first phase amplification reaction mixture, at least a portion of the target nucleic acid sequence of the pre-amplification hybrid isolated in step (b) in a first phase, substantially isothermal, transcription-associated amplification reaction under conditions that support linear amplification thereof, but do not support exponential amplification thereof, thereby resulting in a reaction mixture comprising a first amplification product,
wherein the first phase amplification reaction mixture comprises a non-promoter primer, the non-promoter being complementary to a portion of an extension product of the promoter primer, and comprising a nucleic acid sequence having at least 90% identity to SEQ ID NO: 13, 14, 15, 16, 17, 18, 19, or a complement thereof;
wherein the first amplification product is not a template for nucleic acid synthesis during the first phase, substantially isothermal, transcription-associated amplification reaction;
(d) combining the reaction mixture comprising the first amplification product with an additional promoter primer to produce a second phase amplification reaction mixture,
wherein the second phase amplification reaction mixture additionally comprises a detection oligonucleotide;
(e) performing, in a second phase, a substantially isothermal, transcription-associated amplification reaction in the second phase amplification reaction mixture, an exponential amplification of the first amplification product, thereby synthesizing a second amplification product;
(f) detecting with the detection oligonucleotide at regular time intervals, synthesis of the second amplification product in the second phase amplification reaction mixture; and
(g) quantifying the target nucleic acid sequence in the sample using results from step (f).

16. The method of claim 15, wherein the promoter sequence for the T7 RNA polymerase comprises SEQ ID NO: 65 or 66, or wherein the promoter primer comprises a nucleic acid sequence having at least 90% identity to SEQ ID NO: 4, 5, 6, 7, 8, 9, 10, 11, or 12.

17. The method of claim 15, wherein the non-promoter primer is enzymatically extended in the first phase isothermal transcription-associated amplification reaction.

18. The method of claim 15, wherein the detection oligonucleotide in step (d) is a fluorescently labeled sequence-specific hybridization probe comprising a nucleic acid sequence having at least 90% identity to SEQ ID NO: 20, 21, 22, 23, 24, 25, 26, 27, or 28.

19. The method of claim 15, wherein the method comprises two or more different promoter primers and two or more different non-promoter primers, wherein the two or more different promoter primers and the two or more different non-promoter primers amplify different target nucleic acids to produce two or more different amplification products.

20. The method of claim 19, wherein the different species are Candida species.
